(19) United States
(12) Patent Application Publication
(10) Pub. No.: US 2026/0028554 A1
(43) Pub. Date: Jan. 29, 2026

(54) FRAGRANCE AND FLAVOR COMPOSITIONS COMPRISING AROMATIC DERIVATIVES

(71) Applicant: OSMO LABS, PBC, Cambridge, MA (US)

(72) Inventors: Alexander WILTSCHKO, Somerville, MA (US); Christophe LAUDAMIEL, New York, NY (US); Bradley B. GILBERT, Milford, NJ (US); Andrew P. PATRON, Oceanside, CA (US); Benjamin AMORELLI, Brielle, NJ (US)

(21) Appl. No.: 18/864,440

(22) PCT Filed: Aug. 29, 2024
(86) PCT No.: PCT/US2024/044550

§ 371 Date: Nov. 8, 2024

Related U.S. Application Data
(60) Provisional application No. 63/535,728, filed on Aug. 31, 2023.
(60) Provisional application No. 63/624,739, filed on Jan. 24, 2024.

(51) Int. Cl. C11D 3/16; C07C 69/618; C07C 69/736; C11B 9/00; C11D 3/50

(52) U.S. Cl. CPC C11D 3/164; C07C 69/618; C07C 69/736; C11B 9/0061; C11D 3/50; C07C 2601/10; C07C 2601/14; C07C 2602/08; C07C 2602/10

(57) ABSTRACT

The present application relates to ethyl 3-phenylpropiolate, and related compounds, methods of making them, and methods of using them as flavor and fragrance ingredients in food, cosmetic, pharmaceutical, consumer, and other compositions and products.

### CROSS-REFERENCE TO RELATED APPLICATIONS

This application is an international application which claims priority to, and the benefit of, U.S. Provisional Applic. Ser. No. 63/535,728, filed on Aug. 31, 2023, and U.S. Provisional Applic. Ser. No. 63/624,739, filed on Jan. 24, 2024, the contents of each of which are hereby incorporated by reference in their entireties.

### FIELD OF THE APPLICATION

The present application relates to ethyl 3-phenylpropiolate, and related compounds, methods of making them, and methods of using them as flavor and fragrance ingredients in food, cosmetic, pharmaceutical, consumer, and other compositions and products.

### BACKGROUND

Scent is an important factor used to produce a sense of anticipation, quality, palatability, and security to many consumer products. Flavor is particularly important for foodstuffs. Identifying effective aromas and flavors to impart in a product is an element that contributes to the success of the product, and is useful in product marketing, consumer satisfaction, and consumer retention. Grainy, floral, muguet, orris, fresh, fruity, apple, and pear notes may be particularly desirable for certain flavors and fragrances and may be used in toiletries, cosmetics, household cleaners, room sprays, laundry, and fine fragrance applications, such as in perfumes and toilet water, dental hygiene products (such as toothpastes and mouthwashes), orally administered medications, and food products.

Considerable work is performed by many scientists relating to identifying new substances which can be used, alone or in combinations, to impart to, or enhance, the aroma or flavor of various consumable materials, including, e.g., cosmetics, cleaners, and foodstuffs. While there may be some trends in the relationship between chemical structure and flavor or fragrance such as common use of low molecular weight aldehydes and alcohols as flavors and fragrances—the precise aroma associated with a molecule is exceedingly difficult to predict. Small changes in structure, such a lengthening or shortening a functional group by just one carbon atom, can have profound and unexpected effects on a compound's flavor or fragrance profile. The art of flavor and fragrance prediction is still in its infancy.

Using a proprietary predictive method, the inventors have identified 3-phenylpropiolic acid, as a new ingredient useful in flavors and fragrances. It is believed that this compound has not been previously identified as a flavor or fragrance ingredient, nor has its smell or odor been previously described. The inventors have also identified esters of 3-phenylpropiolic acid, such as ethyl 3-phenylpropiolate, as promising new ingredients useful in flavors and fragrances.

This application describes the surprising and unexpected olfactive qualities of 3-phenylpropiolic acid and its esters (e.g., ethyl 3-phenylpropiolate), and analogs and derivatives thereof, and their use as fragrance and flavor ingredients, and potential applications thereof.

### BRIEF SUMMARY

In one aspect, the application relates to compounds of Formula I:

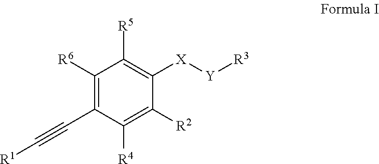

wherein:
   R¹ is CHO, COOH, or COOR^{a};
   R² is H, C₁₋₃ alkyl (e.g., CH₃), OH, CHO, or OC₁₋₃ alkyl (e.g., OCH₃);
   X is selected from —O—, —CH₂—, —C(O)—, —CH(OH)—, and —CH(OR)—;
   Y is selected from —CH₂CH₂CH₂CH₂—, —CH₂CH₂OCH₂—, —CH₂OCH₂CH₂—, —OCH₂CH₂CH₂—, —CH₂CH₂CH₂—, —CH₂CH₂O—, —CH₂OCH₂—, —OCH₂CH₂—, —CH₂CH₂CH₂CH₂CH₂—, —OCH₂CH₂CH₂CH₂—, —CH₂OCH₂CH₂CH₂—, —CH₂CH₂OCH₂CH₂—, —CH₂CH₂CH₂OCH₂—, —CH₂CH₂CH₂CH₂O—, —CH₂CH₂CH₂CH₂CH₂CH₂—, —OCH₂CH₂CH₂CH₂CH₂—, —CH₂OCH₂CH₂CH₂CH₂—, —CH₂CH₂OCH₂CH₂CH₂—, —CH₂CH₂CH₂OCH₂CH₂—, —CH₂CH₂CH₂CH₂OCH₂—, —CH₂CH₂CH₂CH₂CH₂O—, —CH₂CH₂CH₂CH₂CH₂CH₂CH₂—, —OCH₂CH₂CH₂CH₂CH₂CH₂—, —CH₂OCH₂CH₂CH₂CH₂CH₂—, —CH₂CH₂OCH₂CH₂CH₂CH₂—, —CH₂CH₂CH₂OCH₂CH₂CH₂—, —CH₂CH₂CH₂CH₂OCH₂CH₂—, —CH₂CH₂CH₂CH₂CH₂OCH₂—, and —CH₂CH₂CH₂CH₂CH₂CH₂O—,
   wherein said group Y is optionally substituted by one or more groups selected from C₁₋₃ alkyl (e.g., CH₃), OH, CHO, and OC₁₋₃ alkyl (e.g., OCH₃);
   or X—Y is absent;
   R³ is selected from H, R^{b}, OH, OR^{b}, OC(O)R^{b}, CN, COOH, COOR^{b}, C(O)R^{b}, and CHO;
   R⁴, R⁵, and R⁶ are each independently selected from H, C₁₋₃ alkyl (e.g., CH₃), OH, CHO, or OC₁₋₃ alkyl (e.g., OCH₃); and
   each R^{a} and R^{b} is independently optionally substituted C₁₋₆alkyl (e.g., CH₃ or CH₂CH₃), C₂₋₆alkenyl (e.g., allyl or 2-methylprop-1-enyl), CH₂C₃₋₆cycloalkyl (e.g., cyclopropylmethyl), C₃₋₆cycloalkyl (e.g., cyclopropyl), C₄₋₆cycloalkenyl (e.g., cyclopentenyl), optionally substituted phenyl, or optionally substituted benzyl; optionally provided that the compound is not ethyl 3-phenylpropiolate, methyl 3-phenylpropiolate, or 3-phenylpropiolic acid.

In another embodiment, the present disclosure provides a compound of Formula II:

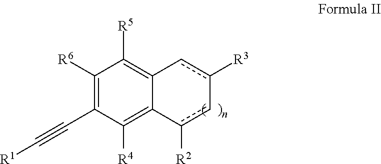

wherein:
   n is 0 or 1;
   R¹ is CHO, COOH, or COOR^{a};
   R² is H, C₁₋₃ alkyl (e.g., CH₃), OH, CHO, or OC₁₋₃ alkyl (e.g., OCH₃);
   R³ is selected from H, R^{b}, OH, OR^{b}, OC(O)R^{b}, CN, COOH, COOR^{b}, C(O)R^{b}, and CHO;
   R⁴, R⁵, and R⁶ are each independently selected from H, C₁₋₃ alkyl (e.g., CH₃), OH, CHO, or OC₁₋₃ alkyl (e.g., OCH₃); and
   each R^{a} and R^{b} is independently optionally substituted C₁₋₆alkyl (e.g., CH₃ or CH₂CH₃), C₂₋₆alkenyl (e.g., allyl or 2-methylprop-1-enyl), CH₂C₃₋₆cycloalkyl (e.g., cyclopropylmethyl), C₃₋₆cycloalkyl (e.g., cyclopropyl), C₄₋₆cycloalkenyl (e.g., cyclopentenyl), optionally substituted phenyl, or optionally substituted benzyl.

In another embodiment, the present disclosure provides a compound of Formula III:

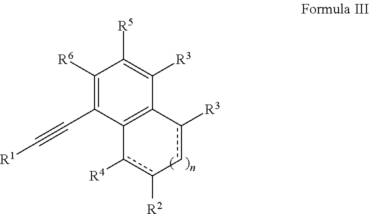

wherein:
   n is 0 or 1;
   R¹ is CHO, COOH, or COOR^{a};
   R² is H, C₁₋₃ alkyl (e.g., CH₃), OH, CHO, or OC₁₋₃ alkyl (e.g., OCH₃);
   R³ is selected from H, R^{b}, OH, OR^{b}, OC(O)R^{b}, CN, COOH, COOR^{b}, C(O)R^{b}, and CHO;
   R⁴, R⁵, and R⁶ are each independently selected from H, C₁₋₃ alkyl (e.g., CH₃), OH, CHO, or OC₁₋₃ alkyl (e.g., OCH₃); and
   each R^{a} and R^{b} is independently optionally substituted C₁₋₆alkyl (e.g., CH₃ or CH₂CH₃), C₂₋₆alkenyl (e.g., allyl or 2-methylprop-1-enyl), CH₂C₃₋₆cycloalkyl (e.g., cyclopropylmethyl), C₃₋₆cycloalkyl (e.g., cyclopropyl), C₄₋₆cycloalkenyl (e.g., cyclopentenyl), optionally substituted phenyl, or optionally substituted benzyl.

In some embodiments, the compound of Formula I is ethyl 3-phenylpropiolate, methyl 3-phenylpropiolate, or 3-phenylpropiolic acid.

In another aspect, the application relates to fragrance and flavor compositions comprising the compound of Formula I or II or III, optionally comprising one or more additives, additional fragrance or flavor ingredients, or a combination of additives and fragrance or flavor ingredients. In some embodiments, the application relates to fragrance and flavor compositions comprising ethyl 3-phenylpropiolate, methyl 3-phenylpropiolate, or 3-phenylpropiolic acid.

In another aspect, the application relates to products, such as consumer products, comprising such fragrance and flavor compositions comprising the compound of Formula I or II, e.g., products comprising ethyl 3-phenylpropiolate, methyl 3-phenylpropiolate, or 3-phenylpropiolic acid.

In another aspect, the present disclosure provides a method of making compound of Formula I or II or III.

The details of one or more embodiments of the application are set forth in the accompanying description below. Unless defined otherwise, all technical and scientific terms used herein have the same meaning as commonly understood by one of ordinary skill in the art to which this application belongs. In the case of conflict, the present specification will control.

Other features and advantages of the application will be apparent from the following detailed description, examples, and claims.

### DETAILED DESCRIPTION

The inventors have unexpectedly found that ethyl 3-phenylpropiolate, and 3-phenylpropiolic acid have alcoholic, fermented, and/or fruity aromas. Such Compounds are therefore potentially useful for products where the inclusion of a pleasing fragrance or flavor is desired, including, but not limited to, perfumes, household products, laundry products, personal care products, cosmetics, dental hygiene products, orally administered medications, and food products. The Compounds of Formula I or II or III may be employed in varying amounts depending upon the specific fragrance or flavor product application, the nature and amount of other flavor or fragrance ingredients present, and the desired aroma and/or flavor of the product.

In a first aspect, the present disclosure provides a compound of Formula I (Compound 1):

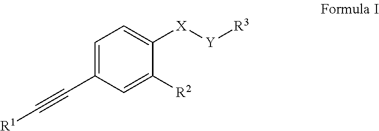

wherein:
   R¹ is CHO, COOH, or COOR^{a};
   R² is H, C₁₋₃ alkyl (e.g., CH₃), OH, CHO, or OC₁₋₃ alkyl (e.g., OCH₃);
   X is selected from —O—, —CH₂—, —C(O)—, CH(OH), and —CH(OR)—;
   Y is selected from —CH₂CH₂CH₂CH₂—, —CH₂CH₂OCH₂—, —CH₂OCH₂CH₂—, —OCH₂CH₂CH₂—, —CH₂CH₂CH₂—, —CH₂CH₂O—, —CH₂OCH₂—, —OCH₂CH₂—, —CH₂CH₂CH₂CH₂CH₂—, —OCH₂CH₂CH₂CH₂—, —CH₂OCH₂CH₂CH₂—, —CH₂CH₂OCH₂CH₂—, —CH₂CH₂CH₂OCH₂—, —CH₂CH₂CH₂CH₂O—, —CH₂CH₂CH₂CH₂CH₂CH₂—, —OCH₂CH₂CH₂CH₂CH₂—, —CH₂OCH₂CH₂CH₂CH₂—, —CH₂CH₂OCH₂CH₂CH₂—, —CH₂CH₂CH₂OCH₂CH₂—, —CH₂CH₂CH₂CH₂OCH₂—, —CH₂CH₂CH₂CH₂CH₂O—, —CH₂CH₂CH₂CH₂CH₂CH₂CH₂—, —OCH₂CH₂CH₂CH₂CH₂CH₂—, —CH₂OCH₂CH₂CH₂CH₂CH₂—, —CH₂CH₂OCH₂CH₂CH₂CH₂—, —CH₂CH₂CH₂OCH₂CH₂CH₂—, —CH₂CH₂CH₂CH₂OCH₂CH₂—, —CH₂CH₂CH₂CH₂CH₂OCH₂—, and —CH₂CH₂CH₂CH₂CH₂CH₂O—,
   wherein said group Y is optionally substituted by one or more groups selected from C₁₋₃ alkyl (e.g., CH₃), OH, CHO, and OC₁₋₃ alkyl (e.g., OCH₃);
   or X—Y is absent;
   R³ is selected from H, R^{b}, OH, OR^{b}, OC(O)R^{b}, CN, COOH, COOR^{b}, C(O)R^{b}, and CHO;
   R⁴, R⁵, and R⁶ are each independently selected from H, C₁₋₃ alkyl (e.g., CH₃), OH, CHO, or OC₁₋₃ alkyl (e.g., OCH₃); and
   each R^{a} and R^{b} is independently optionally substituted C₁₋₆alkyl (e.g., CH₃ or CH₂CH₃), C₂₋₆alkenyl (e.g., allyl or 2-methylprop-1-enyl), CH₂C₃₋₆cycloalkyl (e.g., cyclopropylmethyl), C₃₋₆cycloalkyl (e.g., cyclopropyl), C₄₋₆cycloalkenyl (e.g., cyclopentenyl), optionally substituted phenyl, or optionally substituted benzyl;
      optionally provided that the compound is not ethyl 3-phenylpropiolate, methyl 3-phenylpropiolate, or 3-phenylpropiolic acid.

In further embodiments, of the first aspect, the present disclosure provides:
1.1 Compound 1, wherein the compound is not 3-phenylpropiolic acid;
1.2 Compound 1, wherein the compound is not ethyl 3-phenylpropiolate or methyl 3-phenylpropiolate;
1.3 Compound 1, or 1.2, wherein R¹ is CHO;
1.4 Compound 1, or 1.2, wherein R¹ is COOH;
1.5 Compound 1, or 1.2, wherein R¹ is COOR^{a};
1.6 Compound 1.5, wherein R^{a} is optionally substituted C₁₋₆alkyl (e.g., CH₃ or CH₂CH₃);
1.7 Compound 1.6, wherein R^{a} is selected from methyl, ethyl, propyl, isopropyl, n-butyl, sec-butyl, isobutyl, and tert-butyl;
1.8 Compound 1.5, wherein R^{a} is optionally substituted C₂₋₆alkenyl (e.g., allyl);
1.9 Compound 1.8, wherein R^{a} is allyl or 2-methylprop-1-enyl;
1.10 Compound 1.5, wherein R^{a} is optionally substituted C₃₋₆cycloalkyl (e.g., cyclopropyl);
1.11 Compound 1.5, wherein R^{a} is optionally substituted CH₂C₃₋₆cycloalkyl (e.g., cyclopropylmethyl);
1.12 Compound 1.5, wherein R^{a} is optionally substituted phenyl or optionally substituted benzyl;
1.13 Compound 1, or any of 1.1-1.12, wherein R² is H or OH;
1.14 Compound 1, or any of 1.1-1.12, wherein R² is C₁₋₃ alkyl (e.g., CH₃) or OC₁₋₃ alkyl (e.g., OCH₃), optionally wherein R² is CH₃ or OCH₃;
1.15 Compound 1, or any of 1.1-1.12, wherein R² is CHO;
1.16 Compound 1, or any of 1.1-1.15, wherein X is —O—;
1.17 Compound 1, or any of 1.1-1.15, wherein X is —CH₂—;
1.18 Compound 1, or any of 1.1-1.17, wherein X is —C(O)—;
1.19 Compound 1, or any of 1.1-1.17, wherein X is —CH(OH)—;
1.20 Compound 1, or any of 1.1-1.19, wherein Y is —CH₂CH₂CH₂CH₂—, —CH₂CH₂OCH₂—, —CH₂OCH₂CH₂—, or —OCH₂CH₂CH₂—;
1.21 Compound 1, or any of 1.1-1.19, wherein Y is —CH₂CH₂CH₂—, —CH₂CH₂O—, —CH₂OCH₂—, or —OCH₂CH₂—;
1.22 Compound 1, or any of 1.1-1.19, wherein Y is —CH₂CH₂CH₂CH₂—, or —CH₂CH₂CH₂—;
1.23 Compound 1, or any of 1.1-1.19, wherein Y is —CH₂CH₂CH₂CH₂—;
1.24 Compound 1, or any of 1.1-1.19, wherein X—Y is absent;
1.25 Compound 1, or any of 1.1-1.24, wherein R³ is H;
1.26 Compound 1, or any of 1.1-1.24, wherein R³ is R^{b};
1.27 Compound 1, or any of 1.1-1.24, wherein R³ is OH, OR^{b}, or OC(O)R^{b}, optionally wherein said R^{b} is C₁₋₆ alkyl (e.g., methyl, ethyl, propyl, isopropyl, n-butyl, sec-butyl, isobutyl, or t-butyl);
1.28 Compound 1, or any of 1.1-1.24, wherein R³ is CN, COOH, COOR, C(O)R^{b}, or CHO;
1.29 Compound 1, or any of 1.1-1.28, wherein R^{b} is optionally substituted C₁₋₆alkyl (e.g., CH₃ or CH₂CH₃);
1.30 Compound 1, or any of 1.1-1.28, wherein R^{b} is selected from methyl, ethyl, propyl, isopropyl, n-butyl, sec-butyl, isobutyl, or t-butyl);
1.31 Compound 1, or any of 1.1-1.28, wherein R^{b} is optionally substituted C₃₋₆cycloalkyl;
1.32 Compound 1, or any of 1.1-1.28, wherein R^{b} is cyclopropyl, cyclobutyl, cyclopentyl, or cyclohexyl;
1.33 Compound 1, or any of 1.1-1.28, wherein R^{b} is optionally substituted CH₂C₃₋₆cycloalkyl (e.g., cyclopropylmethyl);
1.34 Compound 1, or any of 1.1-1.28, wherein R^{b} is C₄₋₆cycloalkenyl (e.g., cyclopentenyl, such as cyclopent-1-eneyl);
1.35 Compound 1, or any of 1.1-1.28, wherein R^{b} is optionally substituted phenyl or optionally substituted benzyl;
1.36 Compound 1, or any of 1.1-1.35, wherein the compound of Formula I is a compound of Formula Ia:

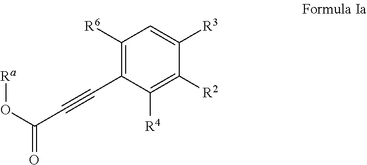

wherein R^{a}, R², R³, R⁴, and R⁶ are as defined in any preceding formula;
1.37 Compound 1.36, wherein R^{a} is selected from methyl, ethyl, propyl, isopropyl, n-butyl, sec-butyl, isobutyl, and tert-butyl;
1.38 Compound 1.36 or 1.37, wherein R², R⁴ and R⁶ are each independently H, C₁₋₃ alkyl (e.g., CH₃) or OC₁₋₃ alkyl (e.g., OCH₃);
1.39 Compound 1.36, 1.37, or 1.38, wherein R³ is R^{b} or OR^{b} and R^{b} is selected from methyl, ethyl, propyl, isopropyl, n-butyl, sec-butyl, isobutyl, tert-butyl, allyl, or 2-methylprop-1-enyl;
1.40 Compound 1.36, 1.37, or 1.38, wherein R³ is R^{b} or OR^{b} and R^{b} is phenyl, cyclopentenyl, or cyclohexenyl;
1.41 Compound 1, or any of 1.1-1.35, wherein the compound of Formula I is a compound of Formula Ib:

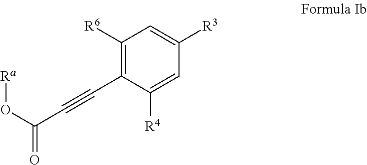

wherein R^{a}, R³, R⁴, and R⁶ are as defined in any preceding formula;
1.42 Compound 1.41, wherein R^{a} is selected from methyl, ethyl, propyl, isopropyl, n-butyl, sec-butyl, isobutyl, and tert-butyl;
1.43 Compound 1.41 or 1.42, wherein R⁴ and R⁶ are each independently H, C₁₋₃ alkyl (e.g., CH₃) or OC₁₋₃ alkyl (e.g., OCH₃);
1.44 Compound 1.41, 1.42, or 1.43, wherein R³ is R^{b} or OR^{b} and R^{b} is selected from methyl, ethyl, propyl, isopropyl, n-butyl, sec-butyl, isobutyl, tert-butyl, allyl, or 2-methylprop-1-enyl;
1.45 Compound 1.41, 1.42, or 1.43, wherein R³ is R^{b} or OR^{b} and R^{b} is phenyl, cyclopentenyl, or cyclohexenyl;
1.46 Compound 1, or any of 1.1-1.35, wherein the compound of Formula I is a compound of Formula Ic:

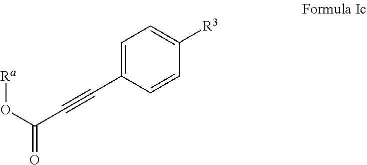

wherein R^{a} and R³ are as defined in any preceding formula;
1.47 Compound 1.36, wherein R^{a} is selected from methyl, ethyl, propyl, isopropyl, n-butyl, sec-butyl, isobutyl, and tert-butyl;
1.48 Compound 1.46 or 1.47, wherein R³ is R^{b} or OR^{b} and R^{b} is selected from methyl, ethyl, propyl, isopropyl, n-butyl, sec-butyl, isobutyl, tert-butyl, allyl, and 2-methylprop-1-enyl;
1.49 Compound 1.46 or 1.47, wherein R³ is R^{b} or OR^{b} and R^{b} is phenyl, cyclopentenyl, or cyclohexenyl
1.50 Compound 1, or any of 1.1-1.35, wherein the compound of Formula I is a compound of Formula Id:

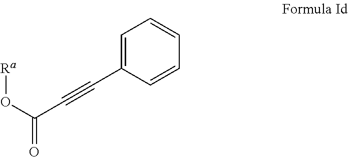

wherein R^{a} is as defined in any preceding formula (e.g., wherein R^{a} is selected from methyl, ethyl, propyl, isopropyl, n-butyl, sec-butyl, isobutyl, and tert-butyl);
1.51 Any preceding compound, wherein the compound has a molecular weight selected from the range of to 172 to 500, or 172 to 450, or 172 to 400, or 172 to 350, or 172 to 300, or 172 to 250, or 172 to 225, or 172 to 200, or 172 to 190;
1.52 Any preceding compound, wherein the compound is selected from:

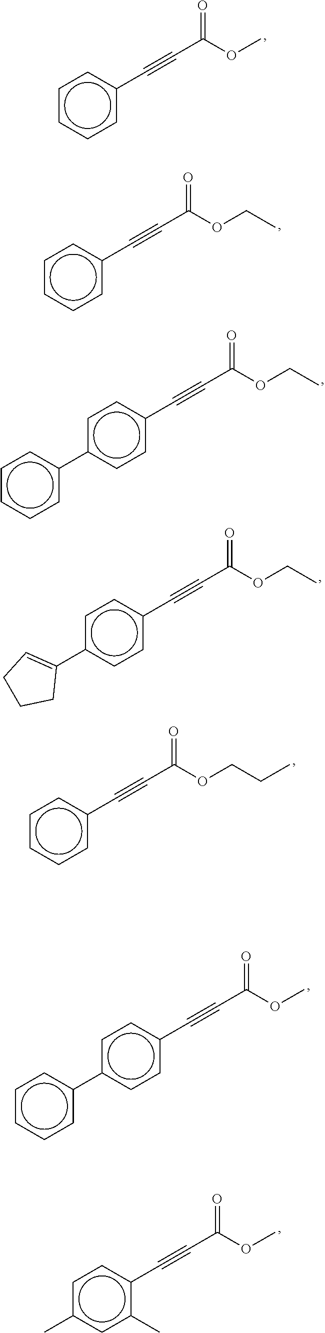

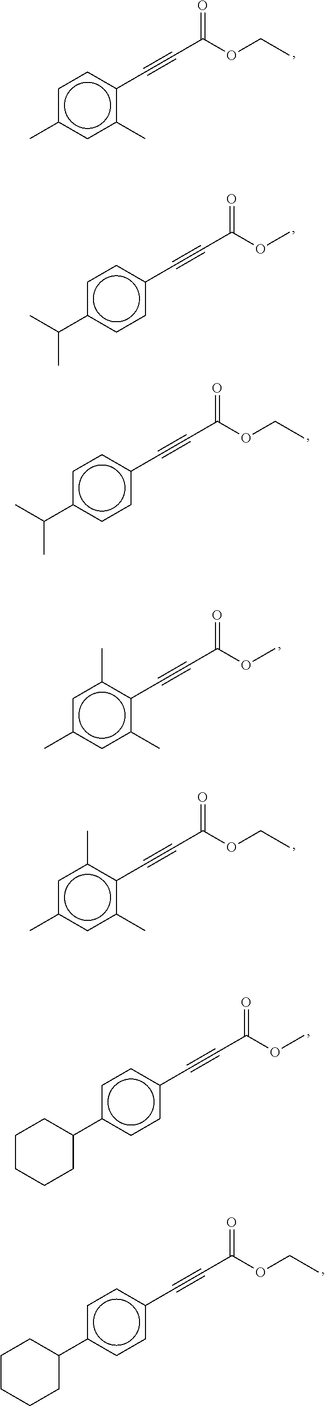

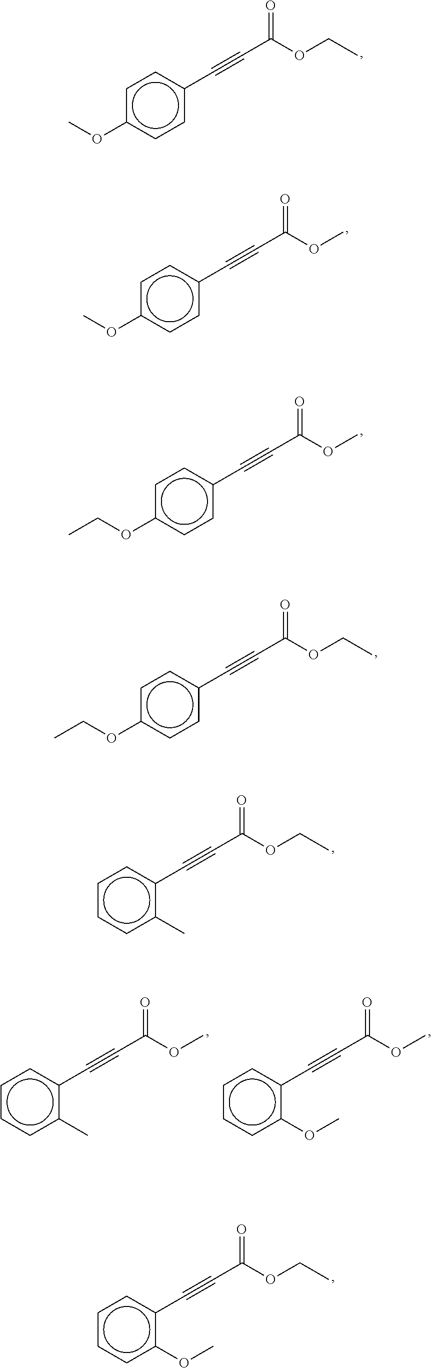

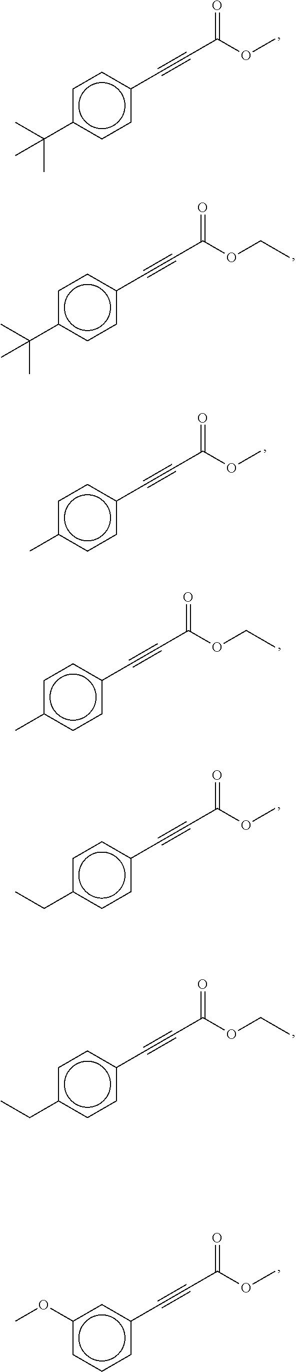

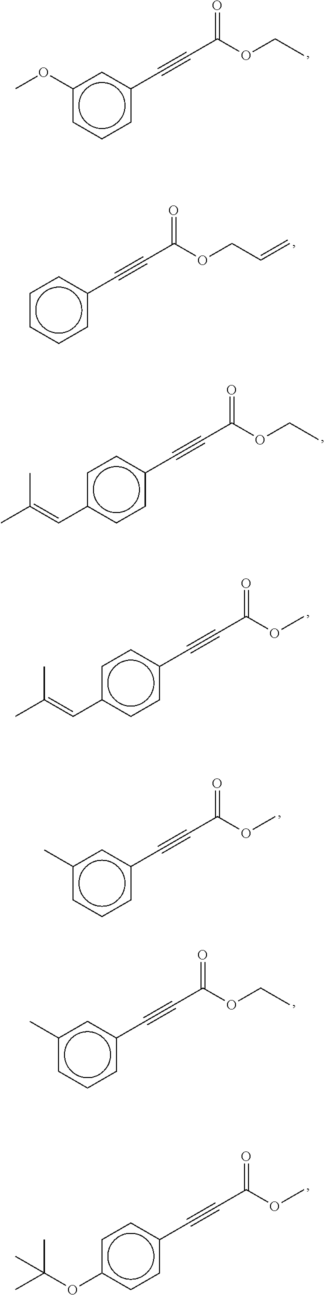

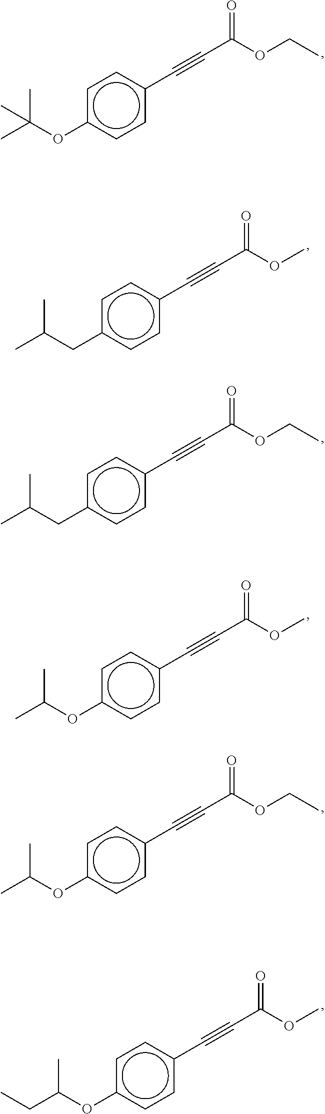

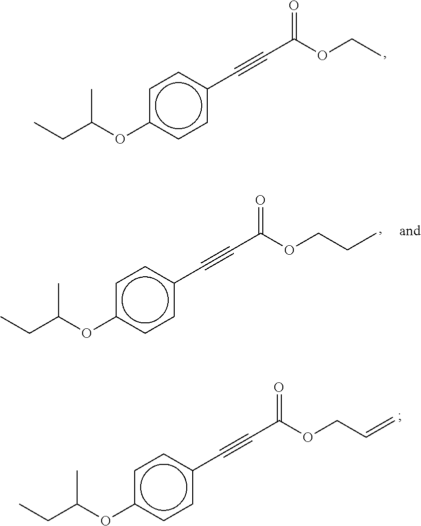

1.53 A compound selected from the group consisting of:

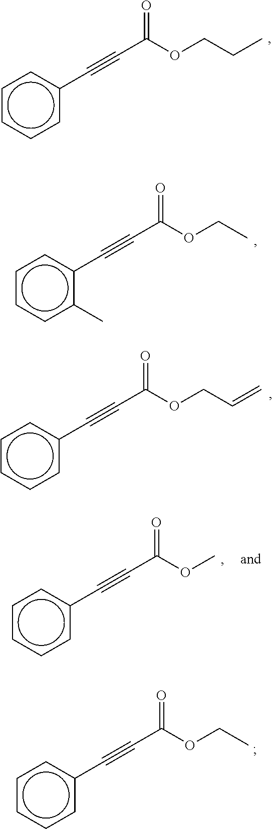

1.54 Any preceding compound wherein if the compound has an acidic or basic atom or functional group, then the compound is in the form of a salt, e.g., a base addition salt or an acid addition salt;
1.55 Any preceding compound, wherein the compound has a pleasing taste and/or aroma, e.g., as judged by a trained flavor or fragrance chemist or master perfumer (e.g., toasted coconut or other nutty aromas and/or tastes).

In another embodiment of the first aspect, the present disclosure provides a compound of Formula II (Compound 2):

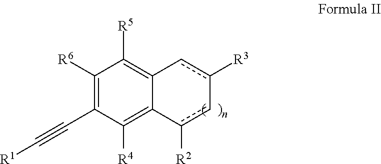

wherein:
   n is 0 or 1;
   R¹ is CHO, COOH, or COOR^{a};
   R² is H, C₁₋₃ alkyl (e.g., CH₃), OH, CHO, or OC₁₋₃ alkyl (e.g., OCH₃);
   R³ is selected from H, R^{b}, OH, OR^{b}, OC(O)R^{b}, CN, COOH, COOR^{b}, C(O)R^{b}, and CHO;
   R⁴, R⁵, and R⁶ are each independently selected from H, C₁₋₃ alkyl (e.g., CH₃), OH, CHO, or OC₁₋₃ alkyl (e.g., OCH₃); and
   each R^{a} and R^{b} is independently optionally substituted C₁₋₆alkyl (e.g., CH₃ or CH₂CH₃), C₂₋₆alkenyl (e.g., allyl or 2-methylprop-1-enyl), CH₂C₃₋₆cycloalkyl (e.g., cyclopropylmethyl), C₃₋₆cycloalkyl (e.g., cyclopropyl), C₄₋₆cycloalkenyl (e.g., cyclopentenyl), optionally substituted phenyl, or optionally substituted benzyl.

In further embodiments, of the first aspect, the present disclosure provides:
2.1. Compound 2, wherein the compound is a compound of Formula IIa:

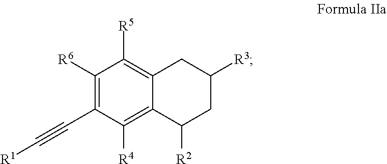

2.2. Compound 2, wherein the compound is a compound of Formula IIb:

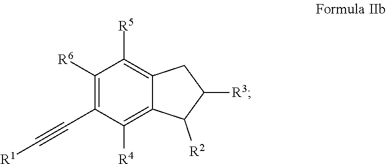

2.3. Compound 2, wherein the compound is a compound of Formula IIc:

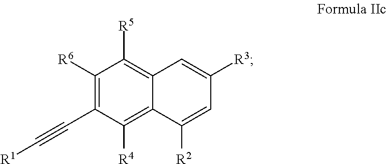

2.4. Compound 2, or any of 2.1-2.3, wherein R¹ is CHO;
2.5. Compound 2, or any of 2.1-2.3, wherein R¹ is COOH;
2.6. Compound 2, or any of 2.1-2.3, wherein R¹ is COOR^{a};
2.7. Compound 2.6, wherein R^{a} is optionally substituted C₁₋₆alkyl (e.g., CH₃ or CH₂CH₃);
2.8. Compound 2.7, wherein R^{a} is selected from methyl, ethyl, propyl, isopropyl, n-butyl, sec-butyl, isobutyl, and tert-butyl;
2.9. Compound 2.6, wherein R^{a} is optionally substituted C₂₋₆alkenyl (e.g., allyl);
2.10. Compound 2.9, wherein R^{a} is allyl or 2-methylprop-1-enyl;
2.11. Compound 2.6, wherein R^{a} is optionally substituted C₃₋₆cycloalkyl (e.g., cyclopropyl);
2.12. Compound 2.6, wherein R^{a} is optionally substituted CH₂C₃₋₆cycloalkyl (e.g., cyclopropylmethyl);
2.13. Compound 2.6, wherein R^{a} is optionally substituted phenyl or optionally substituted benzyl;
2.14. Compound 2, or any of 2.1-2.13, wherein R² is H or OH;
2.15. Compound 2, or any of 2.1-2.13, wherein R² is C₁₋₃ alkyl (e.g., CH₃) or OC₁₋₃ alkyl (e.g., OCH₃), optionally wherein R² is CH₃ or OCH₃;
2.16. Compound 2, or any of 2.1-2.13, wherein R² is CHO;
2.17. Compound 2, or any of 2.1-2.16, wherein R³ is H;
2.18. Compound 2, or any of 2.1-2.16, wherein R³ is R^{b};
2.19. Compound 2, or any of 2.1-2.16, wherein R³ is OH, OR^{b}, or OC(O)R^{b}, optionally wherein said R^{b} is C₁₋₆ alkyl (e.g., methyl, ethyl, propyl, isopropyl, n-butyl, sec-butyl, isobutyl, or t-butyl);
2.20. Compound 2, or any of 2.1-2.16, wherein R³ is CN, COOH, COOR, C(O)R^{b}, or CHO;
2.21. Compound 2, or any of 2.1-2.20, wherein R^{b} is optionally substituted C₁₋₆alkyl (e.g., CH₃ or CH₂CH₃);
2.22. Compound 2, or any of 2.1-2.20, wherein R^{b} is selected from methyl, ethyl, propyl, isopropyl, n-butyl, sec-butyl, isobutyl, or t-butyl);
2.23. Compound 2, or any of 2.1-2.20, wherein R^{b} is optionally substituted C₃. 6cycloalkyl;
2.24. Compound 2, or any of 2.1-2.20, wherein R^{b} is cyclopropyl, cyclobutyl, cyclopentyl, or cyclohexyl;
2.25. Compound 2, or any of 2.1-2.20, wherein R^{b} is optionally substituted CH₂C₃₋₆cycloalkyl (e.g., cyclopropylmethyl);
2.26. Compound 2, or any of 2.1-2.20, wherein R^{b} is C₄₋₆cycloalkenyl (e.g., cyclopentenyl, such as cyclopent-1-eneyl),
2.27. Compound 2, or any of 2.1-2.20, wherein R^{b} is optionally substituted phenyl or optionally substituted benzyl;
2.28. Compound 2, or any of 2.1-2.27, wherein the compound of Formula II is a compound of Formula IId:

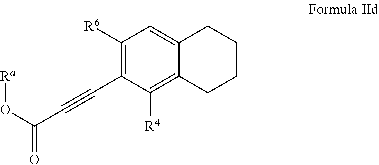

wherein R^{a}, R⁴, and R⁶ are as defined in any preceding formula;
2.29. Compound 2.28, wherein R^{a} is selected from methyl, ethyl, propyl, isopropyl, n-butyl, sec-butyl, isobutyl, and tert-butyl;
2.30. Compound 2.28 or 2.29, wherein R⁴ and R⁶ are each independently H, C₁₋₃ alkyl (e.g., CH₃) or OC₁₋₃ alkyl (e.g., OCH₃);
2.31. Compound 2, or any of 2.1-2.27, wherein the compound of Formula II is a compound of Formula IIe:

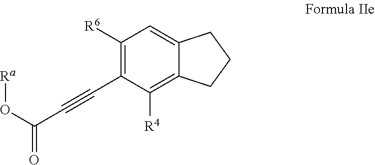

wherein R^{a}, R⁴, and R⁶ are as defined in any preceding formula;
2.32. Compound 2.31, wherein R^{a} is selected from methyl, ethyl, propyl, isopropyl, n-butyl, sec-butyl, isobutyl, and tert-butyl;
2.33. Compound 2.31 or 2.32, wherein R⁴ and R⁶ are each independently H, C₁₋₃ alkyl (e.g., CH₃) or OC₁₋₃ alkyl (e.g., OCH₃);
2.34. Compound 2, or any of 2.1-2.27, wherein the compound of Formula II is a compound of Formula IIf:

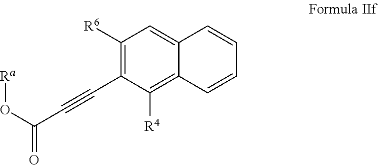

wherein R^{a}, R⁴, and R⁶ are as defined in any preceding formula;
2.35. Compound 2.34, wherein R^{a} is selected from methyl, ethyl, propyl, isopropyl, n-butyl, sec-butyl, isobutyl, and tert-butyl;
2.36. Compound 2.34 or 2.35, wherein R⁴ and R⁶ are each independently H, C₁₋₃ alkyl (e.g., CH₃) or OC₁₋₃ alkyl (e.g., OCH₃);
2.37. Any preceding compound, wherein the compound has a molecular weight selected from the range of to 172 to 500, or 172 to 450, or 172 to 400, or 172 to 350, or 172 to 300, or 172 to 250, or 172 to 225, or 172 to 200, or 172 to 190;
2.38. Any preceding compound, wherein the compound is selected from:

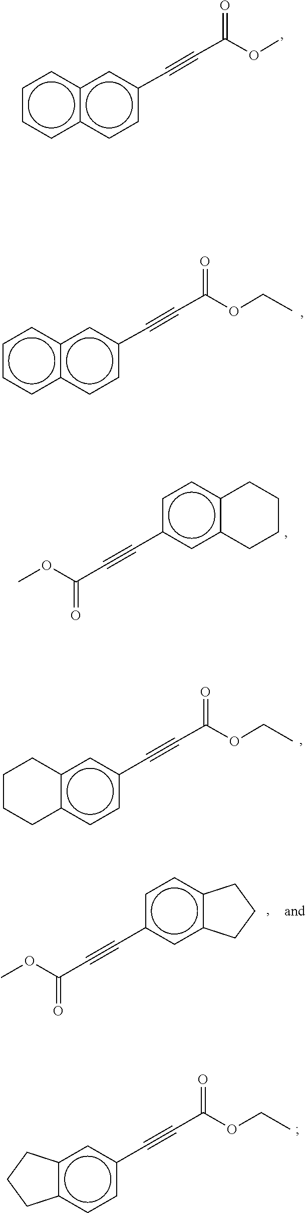

2.39. Any preceding compound wherein if the compound has an acidic or basic atom or functional group, then the compound is in the form of a salt, e.g., a base addition salt or an acid addition salt;
2.40. Any preceding compound, wherein the compound has a pleasing taste and/or aroma, e.g., as judged by a trained flavor or fragrance chemist or master perfumer (e.g., toasted coconut or other nutty aromas and/or tastes).

In another embodiment, the present disclosure provides a compound of Formula III (Compound 3):

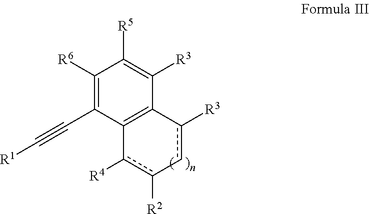

wherein:
   n is 0 or 1;
   R¹ is CHO, COOH, or COOR^{a};
   R² is H, C₁₋₃ alkyl (e.g., CH₃), OH, CHO, or OC₁₋₃ alkyl (e.g., OCH₃);
   R³ is selected from H, R^{b}, OH, OR^{b}, OC(O)R^{b}, CN, COOH, COOR^{b}, C(O)R^{b}, and CHO;
   R⁴, R⁵, and R⁶ are each independently selected from H, C₁₋₃ alkyl (e.g., CH₃), OH, CHO, or OC₁₋₃ alkyl (e.g., OCH₃); and
   each R^{a} and R^{b} is independently optionally substituted C₁₋₆alkyl (e.g., CH₃ or CH₂CH₃), C₂₋₆alkenyl (e.g., allyl or 2-methylprop-1-enyl), CH₂C₃₋₆cycloalkyl (e.g., cyclopropylmethyl), C₃₋₆cycloalkyl (e.g., cyclopropyl), C₄₋₆cycloalkenyl (e.g., cyclopentenyl), optionally substituted phenyl, or optionally substituted benzyl.

In further embodiments, of the first aspect, the present disclosure provides:
3.1. Compound 3, wherein the compound is a compound of Formula IIIa:

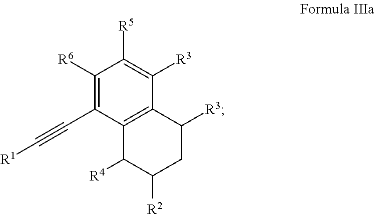

3.2. Compound 3, wherein the compound is a compound of Formula IIIb:

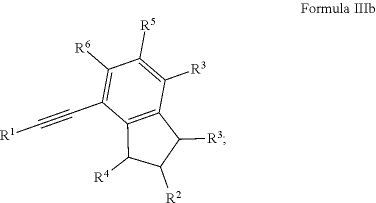

3.3. Compound 3, wherein the compound is a compound of Formula IIIc:

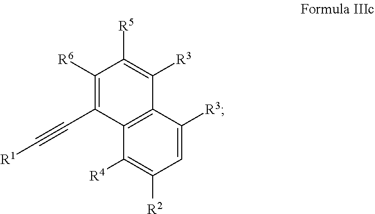

3.4. Compound 3, or any of 3.1-3.3, wherein R¹ is CHO;
3.5. Compound 3, or any of 3.1-3.3, wherein R¹ is COOH;
3.6. Compound 3, or any of 3.1-3.3, wherein R¹ is COOR^{a};
3.7. Compound 3.6, wherein R^{a} is optionally substituted C₁₋₆alkyl (e.g., CH₃ or CH₂CH₃);
3.8. Compound 3.7, wherein R^{a} is selected from methyl, ethyl, propyl, isopropyl, n-butyl, sec-butyl, isobutyl, and tert-butyl;
3.9. Compound 3.6, wherein R^{a} is optionally substituted C₂₋₆alkenyl (e.g., allyl);
3.10. Compound 3.9, wherein R^{a} is allyl or 2-methylprop-1-enyl;
3.11. Compound 3.6, wherein R^{a} is optionally substituted C₃₋₆cycloalkyl (e.g., cyclopropyl);
3.12. Compound 3.6, wherein R^{a} is optionally substituted CH₂C₃₋₆cycloalkyl (e.g., cyclopropylmethyl);
3.13. Compound 3.6, wherein R^{a} is optionally substituted phenyl or optionally substituted benzyl;
3.14. Compound 3, or any of 3.1-3.13, wherein R² is H or OH;
3.15. Compound 3, or any of 3.1-3.13, wherein R² is C₁₋₃ alkyl (e.g., CH₃) or OC₁₋₃ alkyl (e.g., OCH₃), optionally wherein R² is CH₃ or OCH₃;
3.16. Compound 3, or any of 3.1-3.13, wherein R² is CHO;
3.17. Compound 3, or any of 3.1-3.16, wherein R³ is H;
3.18. Compound 3, or any of 3.1-3.16, wherein R³ is R^{b};
3.19. Compound 3, or any of 3.1-3.16, wherein R³ is OH, OR^{b}, or OC(O)R^{b}, optionally wherein said R^{b} is C₁₋₆ alkyl (e.g., methyl, ethyl, propyl, isopropyl, n-butyl, sec-butyl, isobutyl, or t-butyl);
3.20. Compound 3, or any of 3.1-3.16, wherein R³ is CN, COOH, COOR, C(O)R^{b}, or CHO;
3.21. Compound 3, or any of 3.1-3.20, wherein R^{b} is optionally substituted C₁₋₆alkyl (e.g., CH₃ or CH₂CH₃);
3.22. Compound 3, or any of 3.1-3.20, wherein R^{b} is selected from methyl, ethyl, propyl, isopropyl, n-butyl, sec-butyl, isobutyl, or t-butyl);
3.23. Compound 3, or any of 3.1-3.20, wherein R^{b} is optionally substituted C₃. 6cycloalkyl;
3.24. Compound 3, or any of 3.1-3.20, wherein R^{b} is cyclopropyl, cyclobutyl, cyclopentyl, or cyclohexyl;
3.25. Compound 3, or any of 3.1-3.20, wherein R^{b} is optionally substituted CH₂C₃₋₆cycloalkyl (e.g., cyclopropylmethyl);
3.26. Compound 3, or any of 3.1-3.20, wherein R^{b} is C₄₋₆cycloalkenyl (e.g., cyclopentenyl, such as cyclopent-1-eneyl), 3.27. Compound 3, or any of 3.1-3.20, wherein R^{b} is optionally substituted phenyl or optionally substituted benzyl;
3.28. Compound 3, or any of 3.1-3.27, wherein the compound of Formula III is a compound of Formula IIId:

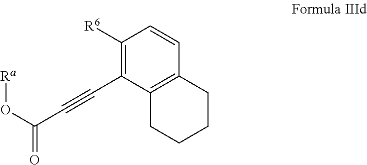

wherein R^{a} and R⁶ are as defined in any preceding formula;
3.29. Compound 3.28, wherein R^{a} is selected from methyl, ethyl, propyl, isopropyl, n-butyl, sec-butyl, isobutyl, and tert-butyl;
3.30. Compound 3.28 or 3.29, wherein R⁶ is H, C₁₋₃ alkyl (e.g., CH₃) or OC₁₋₃ alkyl (e.g., OCH₃);
3.31. Compound 3, or any of 3.1-3.27, wherein the compound of Formula III is a compound of Formula IIIe:

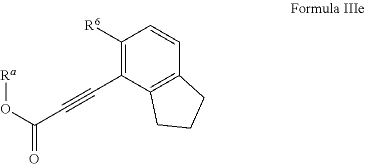

wherein R^{a} and R⁶ are as defined in any preceding formula;
3.32. Compound 3.31, wherein R^{a} is selected from methyl, ethyl, propyl, isopropyl, n-butyl, sec-butyl, isobutyl, and tert-butyl;
3.33. Compound 3.31 or 3.32, wherein R⁶ is H, C₁₋₃ alkyl (e.g., CH₃) or OC₁₋₃ alkyl (e.g., OCH₃);
3.34. Compound 3, or any of 3.1-3.27, wherein the compound of Formula III is a compound of Formula IIIf:

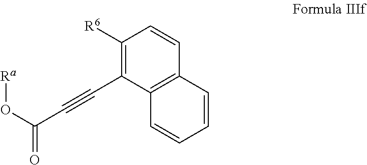

wherein R^{a} and R⁶ are as defined in any preceding formula;
3.35. Compound 3.34, wherein R^{a} is selected from methyl, ethyl, propyl, isopropyl, n-butyl, sec-butyl, isobutyl, and tert-butyl;
3.36. Compound 3.34 or 3.35, wherein R⁶ is H, C₁₋₃ alkyl (e.g., CH₃) or OC₁₋₃ alkyl (e.g., OCH₃);
3.37. Any preceding compound, wherein the compound has a molecular weight selected from the range of to 172 to 500, or 172 to 450, or 172 to 400, or 172 to 350, or 172 to 300, or 172 to 250, or 172 to 225, or 172 to 200, or 172 to 190;
3.38. Any preceding compound, wherein the compound is selected from:

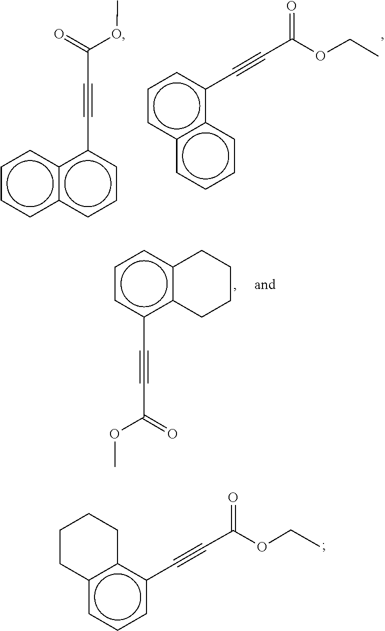

3.39. Any preceding compound wherein if the compound has an acidic or basic atom or functional group, then the compound is in the form of a salt, e.g., a base addition salt or an acid addition salt;
3.40. Any preceding compound, wherein the compound has a pleasing taste and/or aroma, e.g., as judged by a trained flavor or fragrance chemist or master perfumer (e.g., toasted coconut or other nutty aromas and/or tastes).

In a second aspect, the present disclosure provides a flavor composition and/or a fragrance composition (Composition 1) comprising Compound 1, or any of 1.1-1.55, Compound 2, or any of 2.1-2.40, or Compound 3, or any of 3.1-3.40, in admixture with one or more non-toxic, orally acceptable, pharmaceutically acceptable, cosmetically acceptable, or acceptable for a household product, carriers or excipients. In particular embodiments, the second aspect provides:
1.1 Composition 1, wherein the composition is a fragrance composition.
1.2 Composition 1, wherein the composition is a flavor composition.
1.3 Composition 1, or any of Compositions 1.1-1.2, wherein the composition comprises the Compound 1, or any of 1.1-1.55, Compound 2, or any of 2.1-2.40, or Compound 3, or any of 3.1-3.40, in an amount of 0.1 to 100% by weight of the composition, e.g., 0.1 to 90%, or 0.1 to 80%, or 0.1 to 70%, or 0.1 to 60%, or 0.1 to 50%, or 0.1 to 40%, or 0.1 to 30%, or 0.1 to 20%, or 0.1 to 15%, or 0.1 to 10%, or 0.1 to 7.5%, or 0.1 to 5%, or 0.1 to 4%, or 0.1 to 3%, or 0.1 to 2%, or 0.1 to 1%, or 10 to 100%, or 20 to 100%, or 30 to 100%, or 40 to 100%, or 50 to 100%, or 60 to 100%, or 70 to 100%, or 80 to 100%, or 90 to 100%, or 95 to 100%, or 25 to 75%, or 50 to 75%, or 75 to 95%, by weight of the composition.
1.4 Composition 1, or any of Compositions 1.1-1.3, wherein the composition further comprises one or more other flavors or fragrances.
1.5 Composition 1, or any of Compositions 1.1-1.4, wherein the composition further comprises one or more solvents.
1.6 Composition 1.5, wherein the one or more solvents are selected from water, methanol, ethanol, propanol, isopropanol, dimethyl ether, diethyl ether, diisopropyl ether, methyl tert-butyl ether, ethylene glycol, propylene glycol, glycerin, triethyl citrate, triacetin, triglycerides, liquid waxes, propylene glycol derivatives (e.g., polypropylene glycols or ethylene oxide/propylene oxide copolymers), ethylene glycol derivatives (e.g., polyethylene glycols or ethylene oxide/propylene oxide copolymers), other alcohols or ethers, or any combination thereof.
1.7 Composition 1, or any of composition 1.1 to 1.6, wherein the composition is a liquid.
1.8 Composition 1, or any of compositions 1.1 to 1.6, wherein the composition is a soft or waxy solid.
1.9 Composition 1, or any of compositions 1.1-1.8, wherein the composition further comprises one or more of a polymer, gelling agent, powdery substrate, surfactant, emollient, plasticizer, wetting agent, swelling agent, or active agent (e.g., an oral care active or a medicinal active agent), or any other additives as described herein.
1.10 Composition 1, or any of Compositions 1.1-1.9, wherein the composition does not comprise any ingredient or component that would not be safe for ingestion, application to the oral cavity, or topical application to the skin or hair;
1.11 Composition 1, or any of Compositions 1.1-1.10, wherein the composition does not comprise any ingredient or component which is unsafe for, or not approved for, use in a food, cosmetic composition, pharmaceutical composition, oral care composition, or consumer cleaning composition.

As used herein, the term “fragrance composition” means a mixture of fragrance ingredients (e.g., including a Compound of Formula I, Formula II, or Formula III) with one or more non-toxic, cosmetically acceptable, or acceptable for a household product, carriers or excipients, such as solvents. For example, the fragrance ingredient(s) may be dissolved in a suitable solvent or mixed with a powdery substrate, with additional auxiliary substances added (e.g., additives), if desired. A fragrance composition is used, and intended to be used, to provide or impart a desired odor or aroma to a product, such as a cosmetic product or household product (e.g., household cleaners). Thus, a fragrance composition is used as an ingredient or component in a final product, such as a cosmetic product or consumer product, for which a particular fragrance is desired. Examples of products having fragrance compositions include, but are not limited to, perfumes, soaps, insect repellants and insecticides, detergents, household cleaning agents, air fresheners, room sprays, pomanders, candles, cosmetics, toilet waters, pre- and aftershave lotions, talcum powders, hair-care products, body deodorants, anti-perspirants, and pet litter. A fragrance composition should have enough of its fragrance ingredients so that it is effective to provide the desired odor or aroma to the final product, and this depends both on the concentration of the fragrance ingredient(s) in the composition and the concentration of the composition used in the product.

As used herein, the term “flavor composition” means a mixture of flavor ingredients (e.g., including a Compound of Formula I, or Formula II, or Formula III) with one or more non-toxic, orally acceptable, or pharmaceutically acceptable, carriers or excipients, such as solvents. For example, the flavor ingredient(s) may be dissolved in a suitable solvent or mixed with a suitable solid, semi-solid, or liquid excipients, with additional auxiliary substances added (e.g., additives), if desired. A flavor composition is used, and intended to be used, to provide or impart a desired flavor and aroma to a product, such as a food product or oral pharmaceutical product. Thus, a flavor composition is used as an ingredient or component in a final product, such as a food or oral pharmaceutical product, for which a particular flavor is desired. Examples of products having flavor compositions include, but are not limited to, oral care compositions (e.g., dental hygiene products such as mouth wash, toothpaste, floss, and breath fresheners), pharmaceutical compositions (e.g., orally administered medications including liquids, tablets or capsules), and food products. A flavor composition should have enough of its flavor ingredients so that it is effective to provide the desired flavor and aroma to the final product, and this depends both on the concentration of the flavor ingredients in the composition and the concentration of the composition used in the product.

Fragrance and flavor ingredients and mixtures of fragrance and flavor ingredients that may be used in combination with the disclosed compound for the manufacture of fragrance and flavor compositions include, but are not limited to, natural products including extracts, animal products and essential oils, absolutes, resinoids, resins, and concretes, and synthetic fragrance materials which include, but are not limited to, alcohols, aldehydes, ketones, ethers, acids, esters, acetals, phenols, ethers, lactones, furansketals, nitriles, acids, and hydrocarbons, including both saturated and unsaturated compounds and aliphatic carbocyclic and heterocyclic compounds, and animal products. As used herein, the terms “fragrance ingredient” and “flavor ingredient” refer to ingredients other than the Compounds of Formula I which are used to impart a flavor or a fragrance to a composition or product.

Examples of esters which may be used as fragrance ingredients or flavor ingredients in the compositions and products of the present disclosure include, but are not limited to, acrylic acid esters (methyl, ethyl, etc.), acetoacetic acid esters (methyl, ethyl, etc.), anisic acid esters (methyl, ethyl, etc.), benzoic acid esters (allyl, isoamyl, ethyl, geranyl, linalyl, phenylethyl, hexyl, cis-3-hexenyl, benzyl, methyl, etc.), anthranilic acid esters (cinnamyl, cis-3-hexenyl, methyl, ethyl, linalyl, isobutyl, etc.), N-methylanthranilic acid esters (methyl, ethyl, etc.), isovaleric acid esters (amyl, allyl, isoamyl, isobutyl, isopropyl, ethyl, octyl, geranyl, cyclohexyl, citronellyl, terpenyl, linalyl, cinnamyl, phenylethyl, butyl, propyl, hexyl, benzyl, methyl, rhodinyl, etc.), isobutyric acid esters (isoamyl, geranyl, citronellyl, terpenyl, cinnamyl, octyl, nellyl, phenylethyl, phenylpropyl, phenoxyethyl, butyl, propyl, isopropyl, hexyl, benzyl, methyl, ethyl, linalyl, rhodinyl, etc.), undecylenic acid esters (allyl, isoamyl, butyl, ethyl, methyl, etc.), octanoic acid esters (allyl, isoamyl, ethyl, octyl, hexyl, butyl, methyl, linalyl, etc.), octenoic acid esters (methyl, ethyl, etc.), octynecarboxylic acid esters (methyl, ethyl, etc.), caproic acid esters (allyl, amyl, isoamyl, methyl, ethyl, isobutyl, propyl, hexyl, cis-3-hexenyl, trans-2-hexenyl, linalyl, geranyl, cyclohexyl, etc.), hexenoic acid esters (methyl, ethyl, etc.), valeric acid esters (amyl, isopropyl, isobutyl, ethyl, cis-3-hexenyl, trans-2-hexenyl, cinnamyl, phenylethyl, methyl, etc.), formic acid esters (anisyl, isoamyl, isopropyl, ethyl, octyl, geranyl, citronellyl, cinnamyl, cyclohexyl, terpenyl, phenylethyl, butyl, propyl, hexyl, cis-3-hexenyl, benzyl, linalyl, rhodinyl, etc.), crotonic acid esters (isobutyl, ethyl, cyclohexyl, etc.), cinnamic acid esters (allyl, ethyl, methyl, isopropyl, propyl, 3-phenylpropyl, benzyl, cyclohexyl, methyl, etc.), succinic acid esters (monomethyl, diethyl, dimethyl, etc.), acetic acid esters (anisyl, amyl, α-amylcinnamyl, isoamyl, isobutyl, isopropyl, isobornyl, isoeugenyl, eugenyl, 2-ethylbutyl, ethyl, 3-octyl, p-cresyl, o-cresyl, geranyl, α- or β-santalyl, cyclohexyl, cycloneryl, dihydrocuminyl, dimethyl benzyl carbinyl, cinnamyl, styralyl, decyl, dodecyl, terpenyl, guainyl, neryl, nonyl, phenyl ethyl, phenylpropyl, butyl, furfuryl, propyl, hexyl, cis-3-hexenyl, trans-2-hexenyl, cis-3-nonenyl, cis-6-noneyl, cis-3-cis-6-nonadienyl, 3-methyl-2-butenyl, heptyl, benzyl, bornyl, myrcenyl, dihydromyrcenyl, myrtenyl, methyl, 2-methylbutyl, menthyl, linalyl, rhodinyl, etc.), salicylic acid esters (allyl, isoamyl, phenyl, phenylethyl, benzyl, ethyl, methyl, etc.), cyclohexylalkanoic acid esters (ethyl cyclohexylacetate, allyl cyclohexylpropionate, allyl cyclohexylbutyrate, allyl cyclohexylhexanoate, allyl cyclohexyldecanoate, allyl cyclohexylvalerate, etc.), stearic acid esters (ethyl, propyl, butyl, etc.), sebacic acid esters (diethyl, dimethyl, etc.), decanoic acid esters (isoamyl, ethyl, butyl, methyl, etc.), dodecanoic acid esters (isoamyl, ethyl, butyl, etc.), lactic acid esters (isoamyl, ethyl, butyl, etc.), nonanoic acid esters (ethyl, phenylethyl, methyl, etc.), nonenoic acid esters (allyl, ethyl, methyl, etc.), hydroxyhexanoic acid esters (ethyl, methyl, etc.), phenylacetic acid esters (isoamyl, isobutyl, ethyl, geranyl, citronellyl, cis-3-hexenyl, methyl, etc.), phenoxyacetic acid esters (allyl, ethyl, methyl, etc.), furancarboxylic acid esters (ethyl furancarboxylate, methyl furancarboxylate, hexyl furancarboxylate, isobutyl furaneopentyl glycol diacetateropionate, etc.), propionic acid esters (anisyl, allyl, ethyl, amyl, isoamyl, propyl, butyl, isobutyl, isopropyl, benzyl, geranyl, cyclohexyl, citronellyl, cinnamyl, tetrahydrofurfuryl, tricyclodecenyl, heptyl, bornyl, methyl, menthyl, linallyl, terpenyl, α-methylpropionyl, (3-methylpropionyl, etc.), heptanoic acid esters (allyl, ethyl, octyl, propyl, methyl, etc.), heptinecarboxylic acid esters (allyl, ethyl, propyl, methyl, etc.), myristic acid esters (isopropyl, ethyl, methyl, etc.), phenylglycidic acid esters (ethyl phenylglycidate, ethyl 3-methylphenylglycidate, ethyl p-methyl-β-phenylglycidate, etc.), 2-methylbutyric acid esters (methyl, ethyl, octyl, phenyl ethyl, butyl, hexyl, benzyl, etc.), 3-methylbutyric acid esters (methyl, ethyl, etc.), butyric acid esters (anisyl, amyl, allyl, isoamyl, methyl, ethyl, propyl, octyl, guainyl, linallyl, geranyl, cyclohexyl, citronellyl, cinnamyl, nellyl, terpenyl, phenylpropyl, β-phenylethyl, butyl, hexyl, cis-3-hexenyl, trans-2-hexenyl, benzyl, rhodinyl, etc.), and hydroxybutyric acid esters (methyl, ethyl, menthyl or the like of 3-hydroxybutyric acid esters).

Examples of alcohols which may be used as fragrance ingredients or flavor ingredients, or as solvents, in the compositions and products of the present disclosure include, but are not limited to, aliphatic alcohols (isoamyl alcohol, 2-ethylhexanol, 1-octanol, 3-octanol, 1-octene-3-ol, 1-decanol, 1-dodecanol, 2,6-nonadienol, nonanol, 2-nonanol, cis-6-nonenol, trans-2, cis-6-nonadienol, cis-3, cis-6-nonadienol, butanol, hexanol, cis-3-hexenol, trans-2-hexenol, 1-undecanol, heptanol, 2-heptanol, 3-methyl-1-pentanol, etc.); terpene alcohols (borneol, isoborneol, carveol, geraniol, α- or β-santalol, citronellol, 4-thujanol, terpineol, 4-terpineol, nerol, myrcenol, myrtenol, dihydromyrcenol, tetrahydromyrcenol, nerolidol, hydroxycitronellol, farnesol, perilla alcohol, rhodinol, linalool, etc.); and aromatic alcohols (anisic alcohol, α-amylcinnamic alcohol, isopropylbenzylcarbinol, carvacrol, cumin alcohol, dimethylbenzylcarbinol, cinnamic alcohol, phenyl allyl alcohol, phenylethylcarbinol, β-phenylethyl alcohol, 3-phenylpropyl alcohol, benzyl alcohol, etc.).

Examples of aldehydes which may be used as fragrance ingredients or flavor ingredients in the compositions and products of the present disclosure include, but are not limited to, aliphatic aldehydes (acetaldehyde, octanal, nonanal, decanal, undecanal, 2,6-dimethyl-5-heptanal, 3,5,5-trimethylhexanal, cis-3, cis-6-nonadienal, trans-2, cis-6-nonadienal, valeraldehyde, propanal, isopropanal, hexanal, trans-2-hexenal, cis-3-hexenal, 2-pentenal, dodecanal, tetradecanal, trans-4-decenal, trans-2-tridecenal, trans-2-dodecenal, trans-2-undecenal, 2,4-hexadienal, cis-6-nonenal, trans-2-nonenal, 2-methylbutanal, etc.); aromatic aldehydes (anisic aldehyde, α-amylcinnamic aldehyde, α-methylcinnamic aldehyde, cyclamen aldehyde, p-isopropylphenylacetaldehyde, ethylvanillin, cumin aldehyde, salicylaldehyde, cinnamic aldehyde, o-, m- or p-tolylaldehyde, vanillin, piperonal, phenylacetaldehyde, heliotropin, benzaldehyde, 4-methyl-2-pheny-2-pentenal, p-methoxycinnamic aldehyde, p-methoxybenzaldehyde, etc.); and terpene aldehydes (geranial, citral, citronellal, α-sinensal, β-sinensal, perillaldehyde, hydroxycitronellal, tetrahydrocitral, myrtenal, cyclocitral, isocyclocitral, citronellyloxyacetaldehyde, neral, α-methylenecitronellal, myracaldehyde, vernaldehyde, safranal, etc.).

Examples of ketones which may be used as fragrance ingredients or flavor ingredients in the compositions and products of the present disclosure include, but are not limited to, cyclic ketones (1-acetyl-3,3-dimethyl-1-cyclohexene, cis-jasmone, α-, β- or γ-irone, ethyl maltol, cyclotene, dihydronootkatone, 3,4-dimethyl-1,2-cyclopentadione, sotolon, α-, β-, γ- or δ-damascone, α-, β- or γ-damascenone, nootkatone, 2-sec-butylcyclohexanone, maltol, α-, β- or γ-ionone, α-, β- or γ-methylionone, α-, β- or γ-isomethylionone, furaneol, camphor, etc.); aromatic ketones (acetonaphthone, acetophenone, anisylideneacetone, raspberry ketone, p-methyl acetophenone, anisylacetone, p-methoxy acetophenone, etc.); and chain ketones (diacetyl, 2-nonanone, diacetyl, 2-heptanone, 2,3-heptanedione, 2-pentanone, methyl amyl ketone, methyl nonyl ketone, β-methyl naphthyl ketone, methyl heptanone, 3-heptanone, 4-heptanone, 3-octanone, 2,3-hexanedione, 2-undecanone, dimethyloctenone, 6-methyl-5-hepten-2-one, etc.).

Examples of acetals which may be used as fragrance ingredients or flavor ingredients in the compositions and products of the present disclosure include, but are not limited to, acetaldehyde diethyl acetal, acetaldehyde diamyl acetal, acetaldehyde dihexyl acetal, acetaldehyde propylene glycol acetal, acetaldehyde ethyl cis-3-hexenyl acetal, benzaldehyde glycerin acetal, benzaldehyde propylene glycol acetal, citral dimethyl acetal, citral diethyl acetal, citral propylene glycol acetal, citral ethylene glycol acetal, phenylacetaldehyde dimethyl acetal, citronellyl methyl acetal, acetaldehyde phenylethylpropyl acetal, hexanal dimethyl acetal, hexanal dihexyl acetal, hexanal propylene glycol acetal, trans-2-hexenal diethyl acetal, trans-2-hexenal propylene glycol acetal, cis-3-hexenal diethyl acetal, heptanal diethyl acetal, heptanal ethylene glycol acetal, octanal dimethyl acetal, nonanal dimethyl acetal, decanal dimethyl acetal, decanal diethyl acetal, 2-methylundecanal dimethyl acetal, citronellal dimethyl acetal, Ambersage (manufactured by Givaudan), ethyl acetoacetate ethylene glycol acetal, and 2-phenylpropanal dimethyl acetal.

Examples of phenols which may be used as fragrance ingredients or flavor ingredients in the compositions and products of the present disclosure include, but are not limited to, eugenol, isoeugenol, 2-methoxy-4-vinylphenol, thymol, carvacrol, guaiacol, and chavicol, and vanillin.

Examples of ethers and epoxides which may be used as fragrance ingredients or flavor ingredients in the compositions and products of the present disclosure, but are not limited to, anethole, 1,4-cineole, dibenzyl ether, linalool oxide, limonene oxide, nerol oxide, rose oxide, methyl isoeugenol, methyl chavicol, isoamyl phenyl ethyl ether, β-napthyl methyl ether, phenyl propyl ether, p-cresyl methyl ether, vanillyl butyl ether, α-terpinyl methyl ether, citronellyl ethyl ether, geranyl ethyl ether, rose furan, theaspirane, decylmethyl ether, and methylphenyl methyl ether.

Examples of lactones which may be used as fragrance ingredients or flavor ingredients in the compositions and products of the present disclosure include, but are not limited to, γ- or δ-decalactone, γ-heptalactone, γ-nonalactone, γ- or δ-hexylactone, γ- or δ-octalactone, γ- or δ-undecalactone, δ-dodecalactone, δ-2-decenolactone, methyl lactone, 5-hydroxy-8-undecenoic acid δ-lactone, jasmine lactone, menthalactone, dihydrocoumarin, octahydrocoumarin, and 6-methylcoumarin.

Examples of furans which may be used as fragrance ingredients or flavor ingredients in the compositions and products of the present disclosure include, but are not limited to, furan, 2-methylfuran, 3-methylfuran, 2-ethylfuran, 2,5-diethyltetrahydrofuran, 3-hydroxy-2-methyltetrahydrofuran, 2-(methoxymethyl)furan, 2,3-dihydrofuran, furfural, 5-methylfurfural, 3-(2-furyl)-2-methyl-2-propenal, 5-(hydroxymethyl)furfural, 2,5-dimethyl-4-hydroxy-3(2H)-furanone (furaneol), 4,5-dim ethyl-3-hydroxy-2(5H)-furanone (sotolon), 2-ethyl-4-hydroxy-5-methyl-3(2H)-furanone (homofuraneol), 5-ethyl-3-hydroxy-4-methyl-2(5H)-furanone (homosotolon), 3-methyl-1,2-cyclopentanedione (cyclotene), 2(5H)-furanone, 4-methyl-2(5H)-furanone, 5-methyl-2(5H)-furanone, 2-methyl-3(2H)-furanone, 5-methyl-3(2H)-furanone, 2-acetylfuranone, 2-acetyl-5-methylfuran, furfuryl alcohol, methyl 2-furancarboxylate, ethyl 2-furancarboxylate, and furfuryl acetate.

Examples of hydrocarbons which may be used which may be used as fragrance ingredients or flavor ingredients in the compositions and products of the present disclosure include, but are not limited to, α- or β-bisabolene, β-caryophyllene, p-cymene, terpinene, terpinolene, cadinene, cedrene, longifolene, farnesene, limonene, ocimene, myrcene, α- or β-pinene, 1,3,5-undecatriene and valencene.

Examples of acids that may be used which may be used as fragrance ingredients or flavor ingredients in the compositions and products of the present disclosure include, but are not limited to, geranic acid, dodecanoic acid, myristic acid, stearic acid, lactic acid, phenylacetic acid, pyruvic acid, trans-2-methyl-2-pentenoic acid, 2-methyl-cis-3-pentenoic acid, 2-methyl-4-pentenoic acid, and cyclohexanecarboxylic acid.

The fragrance and flavor compositions of the application may comprise as additional fragrance or flavor ingredients one or more natural extracts or oils including, but not limited to, anise, orange, lemon, lime, mandarin, petitgrain, bergamot, lemon balm, grapefruit, elemi, olibanum, lemongrass, neroli, marjoram, angelica root, star anise, basil, bay, calamus, chamomile, caraway, cardamom, cassia, cinnamon, pepper, perilla, cypress, oregano, cascarilla, ginger, parsley, pine needle, sage, hyssop, tea tree, mustard, horseradish, clary sage, clove, cognac, coriander, estragon, eucalyptus, fennel, guaiac wood, dill, cajuput, wormseed, pimento, juniper, fenugreek, garlic, laurel, mace, myrrh, nutmeg, spruce, geranium, citronella, lavender, lavandin, palmarosa, rose, rosemary, sandalwood, oakmoss, cedarwood, vetiver, linaloe, bois de rose, patchouli, labdanum, cumin, thyme, ylang ylang, birch, capsicum, celery, tolu balsam, genet, immortelle, benzoin, jasmine, cassie, tuberose, reseda, marigold, mimosa, opoponax, orris, vanilla and licorice. Each of these natural extracts or oils comprises a complex mixture of chemical compounds, which may include those compounds described above. Additional fragrance ingredients may be isolated from natural products, for example, geraniol and citronellal may be isolated from citronella oil, citral may be isolated from lemon-grass oil, eugenol may be isolated from clove oil, and linalool may be isolated from rosewood oil. Animal products used in fragrance compositions include, but are not limited to, musk, ambergris, civet and castoreum. The natural ingredients described herein may also be produced synthetically, and may include the compounds disclosed herein, and be used as fragrance and/or flavor ingredients in the fragrance and flavor compositions of the present application.

Examples of fragrance ingredients used in perfumes, air fresheners, laundry detergents, pet litters, cleaning products, liquid and bar soaps, shampoos and conditioners, cosmetics, deodorants, and personal hygiene products include, but are not limited to: hexyl cinnamic aldehyde; amyl cinnamic aldehyde; amyl salicylate; hexyl salicylate; terpineol; 3,7-dimethyl-cis-2,6-octadien-1-ol; 2,6-dimethyl-2-octanol; 2,6-dimethyl-7-octen-2-ol; 3,7-dimethyl-3-octanol; 3,7-dimethyl-trans-2,6-octadien-1-ol; 3,7-dimethyl-6-octen-1-ol; 3,7-dimethyl-1-octanol; 2-methyl-3-(para-tert-butylphenyl)-propionaldehyde; 4-(4-hydroxy-4-methylpentyl)-3-cyclohexene-1-carboxaldehyde; tricyclodecenyl propionate; tricyclodecenyl acetate; anisaldehyde; 2-methyl-2-(para-iso-propylphenyl)-propionaldehyde; ethyl-3-methyl-3-phenyl glycidate; 4-(para-hydroxyphenyl)-butan-2-one; 1-(2,6,6-trimethyl-2-cyclohexen-1-yl)-2-buten-1-one; para-methoxyacetophenone; para-methoxy-alpha-phenylpropene; methyl-2-n-hexyl-3-oxo-cyclopentane carboxylate; undecalactone gamma, geraniol; geranyl acetate; linalool; linalyl acetate; tetrahydrolinalool; citronellol; citronellyl acetate; dihydromyrcenol; dihydromyrcenyl acetate; tetrahydromyrcenol; terpinyl acetate; nopol; nopyl acetate; 2-phenylethanol; 2-phenylethyl acetate; benzyl alcohol; benzyl acetate; benzyl salicylate; benzyl benzoate; styrallyl acetate; dimethylbenzylcarbinol; trichloromethylphenylcarbinyl methylphenylcarbinyl acetate; isononyl acetate; vetiveryl acetate; vetiverol; 2-methyl-3-(p-tert-butylphenyl)-propanal; 2-methyl-3-(p-isopropylphenyl)-propanal; 3-(p-tert-butylphenyl)-propanal; 4-(4-methyl-3-pentenyl)-3-cyclohexenecarbaldehyde; 4-acetoxy-3-pentyltetrahydropyran; methyl dihydrojasmonate; 2-n-heptylcyclopentanone; 3-methyl-2-pentyl-cyclopentanone; n-decanal; n-dodecanal; 9-decenol-1; phenoxyethyl isobutyrate; phenylacetaldehyde dimethylacetal; phenylacetaldehyde diethylacetal; geranonitrile; citronellonitrile; cedryl acetal; 3-isocamphylcyclohexanol; cedryl methyl ether; isolongifolanone; aubepine nitrile; aubepine; heliotropine; eugenol; vanillin; diphenyl oxide; hydroxycitronellal ionones; methyl ionones; isomethyl ionones; irones; cis-3-hexenol and esters thereof; indane musk fragrances; tetralin musk fragrances; isochroman musk fragrances; macrocyclic ketones; macrolactone musk fragrances; and ethylene brassylate.

The fragrance and flavor ingredients in a given product's fragrance or flavor composition are selected based on the intended use of the product and the product's desired aroma. For example, flavor ingredients used in toothpaste, mouth wash, and dental hygiene products may be selected to impart “freshness” and include, but are not limited to, spearmint oil, peppermint oil, star anise oil, lemon oil, and menthol.

Flavor compositions may be used to mask the unpleasant taste of orally administered medications. For example, if a medication is salty, a flavor composition that has cinnamon, raspberry, orange, maple, butterscotch, or glycyrrhiza (licorice) flavor may be used to mask the taste. If a medication is overly sweet, a flavor composition that has a berry, vanilla, or acacia flavor may render the medication more palatable. In the case of bitter tasting medications, flavor compositions that have cocoa, chocolate-mint, wild cherry, walnut, glycyrrhiza (licorice), and eriodictyon flavors might be used, whereas sour medications may be improved by flavor compositions that have fruity, citrus, or cherry flavors. These flavors may be provided by the natural or synthetic flavor ingredients discussed herein.

Examples of flavor ingredients used in flavor compositions for food products also include, but are not limited to, glucosyl steviol glycosides, isomenthols, carbonothoic acids, cassyrane, 1,5-octadien-3-ol, 2-mercaptoheptan-4-ol, 4 3-(methylthio)decanal, (4Z,7Z)-trideca-4,7-dienal, Persicaria odorata oil, Amacha leaves extract, glutamyl-2-aminobutyric acid, glutamyl-2-aminobutyric acid, glutamyl-norvalyl-glycine, glutamyl-norvaline, N1-(2,3-Dimethoxybenzyl)-N2-(2-(pyridin-2-yl)ethyl) oxalamide, 1-(2-hydroxy-4-methylcyclohexyl)ethanone, Mexican lime oil, Persian lime oil, 6-methoxy-2,6-dimethylheptanal, 3,5-undecadien-2-one, 2,5-undecadien-1-ol, triethylthialdine. 4-methylpentyl 4-methylvalerate, (R)—N-(1-methoxy-4-methylpentan-2-yl)-3,4-dimethylbenzamide, 2 N-acetyl glutamate, 1,3-propanediol, Szechuan pepper extract, Tasmannia lanceolata extract, Mentha longifolia oil, mangosteen distillate, ethyl 3-(2-hydroxyphenyl)propanoate, 1-cyclopropanemethyl-4-methoxybenzene, prenyl thioisobutyrate, prenyl thioisovalerate, matairesinol, stevioside, 1-(2,4-dihydroxyphenyl)-3-(3-hydroxy-4-methoxyphenyl)propan-1-one, ethyl 5-formyloxydecanoate, 3-[3-(2-isopropyl-5-methyl-cyclohexyl)ureido]butyric acid ethyl ester, 2-Isopropyl-4-methyl-3-thiazoline, 2,6,10-trimethyl-9-undecenal, 5-mercapto-5-methyl-3-hexanone, Meyer lemon oil, teviol glycoside extract, Stevia rebaudiana, rebaudioside A 60%, rubescenamine, 4-amino-5-(3-(isopropylamino)-2,2-dimethyl-3-oxopropoxy)-2-methylquinoline-3-carboxylic acid, 3-methyl-5-(2,2,3-trimethylcyclopent-3-en-1-yl)pent-4-en-2-ol, (1-Methyl-2-(1,2,2-trimethylbicyclo[3.1.0]hex-3-ylmethyl)cyclopropyl)methanol, erospicata oil, and curly mint oil. See L. J. Marnett et al., GRAS Flavoring Substances 26, Food Technology, 44-45 (2013).

Preferred solvents and excipients for use in the compositions and products of the present disclosure include, but are not limited to, triethyl citrate, triacetin, glycerol, propylene glycol, dipropylene glycol, isopropyl myristate, ethanol, water, triglycerides, liquid waxes, propylene glycol derivatives (e.g., polymers), and ethylene glycol derivatives (e.g., polymers).

The amount of a given fragrance or flavor ingredient in a fragrance or flavor composition cannot be categorically described because it varies depending on the type product being scented or flavored, the intended use of the product, and the desired aroma and/or taste of the product. The amount of a fragrance or flavor ingredient in a fragrance or flavor composition is usually in the range of from about 1% to about 99% by mass of the fragrance composition. When the amount of the ingredient is too small, a sufficient strength of the scent or flavor may not be obtained. Further, when the amount of the ingredient is too large, a larger amount of the agent(s) needed to solubilize the ingredient may be needed, which may in turn reduce the desired aromatic or flavor properties of the end product by inhibiting volatilization or other mechanisms by which the flavor or fragrance is dispersed when the product is used or consumed. The amount of each of the fragrance and flavor ingredients in a given fragrance or flavor composition must therefore be selected based upon the aromatic and/or flavor characteristics of the selected ingredient, the overall composition of the product, and the intended aromatic and/or flavor effect.

Additives may be used in the flavor and fragrance compositions of the present disclosure. Additives that may be used include, but are not limited to, solvents, surfactants, pH adjusters, buffers, thickening agents, desiccants, emulsifiers, foaming agents, stabilizers, antioxidants, and disintegrating agents. Other fragrance and flavor composition additives will be selected in accordance with the intended use of the composition.

Solvents, for example water-soluble organic solvents, which may be used in the flavor and fragrance compositions of the present disclosure include, but are not limited to, ethanol, propanol, isopropanol, butanol, 3-methoxy-3-methyl-1-butanol, benzyl alcohol, ethyl carbitol (diethylene glycol monoethyl ether), ethylene glycol, propylene glycol, dipropylene glycol, butylene glycol, hexylene glycol, glycerin, ethylene glycol monomethyl ether, propylene glycol monomethyl ether, propylene glycol monoethyl ether, and dipropylene glycol monomethyl ether. These water-soluble solvents may be used solely or in combination. The content of the water-soluble organic solvent in the compositions of the application may be determined according to the desired composition properties, and is usually from about 1% to about 99% by mass.

Oil-soluble organic solvents which may be used with the flavor and fragrance compositions of the application include, but are not limited to, isoparaffin, paraffin, limonene, pinene, triethyl citrate, benzyl benzoate, isopropyl myristate, triacetin, and silicone.

Preferred solvents include, but are not limited to, triethyl citrate, triacetin, glycerol, ethanol, water, triglycerides, liquid waxes, propylene glycol derivatives, and ethylene glycol derivatives.

In some embodiments, the flavor and fragrance compositions and products of the present disclosure may further comprise other substances, including, but not limited to, sequestering agents, preservatives, antioxidants, deodorizers, sterilization agents, ultraviolet absorbers, pH adjusters, insecticidal components, components for protection from insects, insect repellents, colorants, excipients, and buffers. The substances used in, or in addition to, the fragrance and flavor compositions of the present application may be determined by the product in which the composition is included. When the substance is used in a flavor or fragrance composition, it may be an additive. When the substance is used alongside a flavor or fragrance composition, it may be considered as part of a product composition that comprises a fragrance or flavor composition.

Excipients that may be used in the fragrance and flavoring compositions and products of the present disclosure may vary depending on the use of the intended product and its overall composition. In some instances, the excipient may be included in the fragrance or flavor composition or may, alternatively, be independent of the composition. Excipients used in or with flavoring compositions of an orally administered medication may include, but are not limited to, tablet coatings, such as a cellulose ether hydroxypropyl methylcellulose, synthetic polymer, shellac, corn protein zein or other polysaccharides, and gelatin. In contrast, cosmetic excipients may include, but are not limited to, Carbopol 940 ETD, triethanolamine, purified water, glycerin, imidazolidinyl urea, EDTA, polyvinyl alcohol, methyl parabens phenoxyethanol 0, ethyl alcohol 1, peg 7 glyceryl cocoate, peg 6 triglyceryl caproic glycerides, acemulogar LAM V, isopropyl myristate, tegosoft CT, xanthan gum, sepicide CL, polyquaternium 7, and Vaseline oils. Additional suitable excipients for use with or in a flavor and/or fragrance composition for a given product will be readily selected by those having ordinary skill in the art.

Buffers that may be used with the fragrance and flavoring compositions of the present application may vary depending on the use of the intended product and its overall composition. In some instances, the buffer may be included in the fragrance or flavor composition or may, alternatively, be independent of the composition. Examples of buffers that may be used in or with the fragrance and flavor compositions of the application include, but are not limited to, citrates, acetates, and phosphates. For example, trisodium citrate may be used as a flavor or as a preservative, and is known to impart tartness to a flavor, but also acts as a buffer. Trisodium citrate is an ingredient in a variety of sodas and other beverages, as well as drink mixes and bratwurst. In cosmetic products, disodium hydrogen phosphate, potassium dihydrogen phosphate, disodium hydrogen phosphate and, and citric acid may be used to buffer the pH of the product. In toothpaste, calcium carbonate and/or dicalcium phosphate may be used as pH buffers. Additional suitable buffers for use with or in a flavor and/or fragrance composition for a given product will be readily selected by those having ordinary skill in the art.

In a third aspect, the present disclosure provides a product which comprises Compound 1, or any of 1.1-1.55, Compound 2, or any of 2.1-2.40, or Compound 3, or any of 3.1-3.40, or Composition 1 or any of 1.1 to 1.11. In some embodiments, the product may be selected from the following: personal care products (e.g., a soap, skin cream or lotion, balm, shampoo, body wash, shower gel, hydrating cream, deodorant, antiperspirant, after-shave lotion, cologne, perfume, or other hair care or skin care product), sunscreens, insect repellants and insecticides, detergents, household cleaning agents (e.g., a surface cleaner, a metal cleaner, a wood cleaner, a glass cleaner, a body cleaner such as a soap, a dish-washing detergent, or a laundry detergent), air fresheners, room sprays, pomanders, candles, cosmetics (e.g., perfumes, colognes, nail polish, eye liner, mascara, lipstick, foundation, concealer, blush, bronzer, eye shadow, lip liner, lip balm), toilet waters, talcum powders, and pet litter.

Having now described some embodiments of the application, it should be apparent to those skilled in the art that the foregoing is merely illustrative and not limiting, having been presented by way of example only. The embodiments of the application can therefore be in other specific forms without departing from the spirit or essential characteristics thereof.

Those skilled in the art should recognize or be able to ascertain, using no more than routine experimentation, equivalents to the specific embodiments of the application. It is therefore to be understood that the embodiments described herein are presented by way of example only and that the scope of the application is thus indicated by the appended claims and equivalents thereto, and that the application may be practiced otherwise than as specifically described in the foregoing description.

The term “about,” when used to describe one of the compositions of the application, refers to a recited percentage ±5%, ±4%, ±3%, ±2.5%, ±2%, ±1.5%, ±1%, ±0.75%, ±0.5%, ±0.25%, or ±0.1%. In one embodiment, the term “about,” refers to a recited percentage ±5%. For example, “about 50%” refers to the range 45% to 55%. In one embodiment, the term “about,” refers to a recited percentage ±2.5%. In one embodiment, the term “about,” refers to a recited percentage ±1%. In one embodiment, the term “about,” refers to a recited percentage ±0.5%. In one embodiment, the term “about,” refers to a recited percentage ±0.1%.

As used herein, the singular forms “a,” “an,” and “the” include plural referents unless the context clearly dictates otherwise. Thus, for example, reference to “a fragrance ingredient” includes not only a single fragrance ingredient but also a combination or mixture of two or more different fragrance ingredients, reference to “an additive” includes a single additive as well as two or more additives, and the like.

As used herein, the phrases “for example,” “for instance,” “such as,” or “including” are meant to introduce examples that further clarify more general subject matter. These examples are provided only as an aid for understanding the disclosure, and are not meant to be limiting in any fashion. Furthermore, as used herein, the terms “may,” “optional,” “optionally,” or “may optionally” mean that the subsequently described circumstance may or may not occur, so that the description includes instances where the circumstance occurs and instances where it does not. For example, the phrase “optionally present” means that an object may or may not be present, and, thus, the description includes instances wherein the object is present and instances wherein the object is not present.

As used herein, “optionally substituted” means that the indicated core or functional group is either unsubstituted or substituted by one or more groups up to the maximum permitted by the rules of valency, wherein said groups are selected from: halo, hydroxy, cyano, C₁₋₆alkyl, C₂₋₆alkenyl, C₂₋₆alkynyl, C₃₋₆cycloalkyl, C₁₋₆haloalkyl, C₁₋₆-alkoxy, —O—Si(R^{x})₃, —O—R^{x}, —C(O)H, —C(O)—R^{x}, —C(O)—O—R^{x}, —C(O)—NH—R^{x}, —C(O)—N—(R^{x})(R^{x}), —O—C(O)—R^{x}, —NH(R^{x})—C(O)—R^{x}, —N(R^{x})(R^{x})—C(O)—R^{x}), —NH(R^{x}), —N(R^{x})(R^{x}), heterocycloalkyl, aryl, and heteroaryl; wherein each of said C₁₋₆alkyl, C₃₋₆cycloalkyl, heterocycloalkyl, aryl or heteroaryl is further optionally substituted by one or more halo, hydroxy, cyano, C₁₋₆alkyl, C₂₋₆alkenyl, C₂₋₆alkynyl, C₃₋₆cycloalkyl, C₁₋₆haloalkyl, —O—Si(R^{x})₃, —O—R^{x}, —C(O)H, —C(O)—R^{x}, —C(O)—O—R^{x}, —C(O)—NH—R^{x}, —C(O)—N—(R^{x})(R^{x}), —O—C(O)—R^{x}, —NH(R^{x})—C(O)—R^{x}, —N(R^{x})(R^{x})—C(O)—R^{x}), —NH(R^{x}), —N(R^{x})(R^{x}), heterocycloalkyl, aryl, and heteroaryl; and wherein each R^{x} is independently selected from hydrogen, C₁₋₆alkyl, C₂₋₆alkenyl, C₂₋₆alkynyl, C₃₋₆cycloalkyl, heterocycloalkyl, aryl and heteroaryl.

As used herein, the term “C₁₋₆-alkyl” means a saturated linear or branched free radical consisting essentially of 1 to 6 carbon atoms and a corresponding number of hydrogen atoms. Exemplary C₁₋₆-alkyl groups include methyl, ethyl, n-propyl, isopropyl, n-butyl, isobutyl, sec-butyl, tert-butyl, n-pentyl, isopentyl, neopentyl, n-hexyl, and thexyl. Other C₁₋₆-alkyl groups will be readily apparent to those of skill in the art given the benefit of the present disclosure. The terms “C₁₋₃-alkyl”, “C₁₋₄-alkyl”, etc., have equivalent meanings, i.e., saturated linear or branched free radical consisting essentially of 1 to 3 (or 4) carbon atoms and a corresponding number of hydrogen atoms. The similar terms “C₂₋₆-alkenyl,” “C₂₋₆-alkynyl,” “C₃₋₆-cycloalkyl,” “C₁₋₆-haloalkyl,” “C₁₋₆-alkoxy,” and the like, refer to corresponding functional groups having the stated number of carbon atoms, wherein “alkenyl” refers to an unsaturated linear or branched free radical having at least one double bond, “alkynyl” refers to an unsaturated linear or branched free radical having at least one triple bond, “haloalkyl” refers to an alkyl radical having at least one halogen atom attached to a carbon atom, and “alkoxy” refers to an alkyl radical having at least one oxygen atom attached to the alkyl radical and wherein the attachment point of the functional group is through the oxygen (i.e., to form an ether). Exemplary alkenyl groups include vinyl and allyl. Exemplary alkynyl groups include ethynyl and propynyl. Exemplary haloalkyl groups include fluoromethyl, difluoromethyl, trifluoromethyl, 3,3,3-trifluorethyl, and like groups with chlorine, bromine or iodine. “Cycloalkyl” refers to a carbocyclic ring attached via a ring carbon atom. Exemplary cycloalkyl groups include cyclopropyl, cyclobutyl, cyclopentyl and cyclohexyl.

As used herein, the term “heteroaryl” means an aromatic free radical having 5 to 20 atoms (i.e., ring atoms) that form a ring, wherein at least one atom (e.g., 1 to 5) of the ring atoms are carbon and at least one atom of the remaining ring atoms is a nitrogen, sulfur, or oxygen. Heteroaryl rings include monocyclic, bicyclic fused, and polycyclic fused ring systems provided that at least one ring of the ring system has at least one heteroatom (N, S, or O), and all rings are aromatic. Exemplary 5-membered heteroaryl groups include furyl, thienyl (thiophenyl), pyrrolyl, oxazolyl, thiazolyl, pyrazolyl, isothiazolyl, isoxazolyl, imidazolyl, triazolyl, oxadiazolyl, thiadiazolyl, and tetrazolyl. Exemplary 6-membered heteroaryl groups include pyridyl, pyrimidyl, pyrazinyl, pyridazinyl, and 1,2,4-triazinyl. Exemplary fused heteroaryl groups include benzoxazolyl, benzothiazolyl, benzisothiazolyl, benzisoxazolyl, benzimidazolyl, indolyl, quinolinyl, isoquinolinyl, quinazolinyl, and quinoxalinyl. Other heteroaryl groups will be readily apparent to those of skill in the art given the benefit of the present disclosure. In general, the heteroaryl group typically is attached to the main structure via a carbon atom. However, those of skill in the art will realize that certain other atoms, e.g., hetero ring atoms, can be attached to the main structure.

As used herein, the term “aryl” means an aromatic free radical having 5 or 6 atoms (i.e., ring atoms) that form a ring, wherein all of the ring atoms are carbon. Exemplary aryl groups include phenyl and naphthyl.

As used herein, the term “heterocycloalkyl” means an aromatic free radical having 3 to 20 atoms (i.e., ring atoms) that form a ring, wherein at least one atom (e.g., 1 to 5) of the ring atoms are carbon and at least one atom of the remaining ring atoms is a nitrogen, sulfur, or oxygen, and wherein at least one ring is non-aromatic. Heterocycloalkyl rings include monocyclic, bicyclic fused, bicyclic spiro-joined, polycyclic bridged, and polycyclic fused ring systems, provided that at least one ring of the ring system has at least one heteroatom (N, S, or O) and at least one ring of the ring system is non-aromatic (e.g., saturated). Exemplary saturated heterocycloalkyl groups include azetidinyl, aziridinyl, oxiranyl, oxetanyl, tetrahydrofuranyl, pyrrolidinyl, piperidinyl, piperazinyl, morpholinyl, and thiomorpholinyl. Heterocycloalkyl rings systems include ring systems in which an aromatic ring is fused to a nonaromatic ring, such as will be obtained by partial reduction of a polycyclic aromatic ring system. Exemplary ring systems of this category include indolinyl, tetrahydroquinolinyl, and tetrahydroisoquinolinyl. Other heterocycloalkyl groups will be readily apparent to those of skill in the art given the benefit of the present disclosure. A heterocycloalkyl groups can be attached to the main structure either through a carbon atom or a nitrogen atom of the ring.

The term “compound,” as used herein, unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives thereof where applicable, in context. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. The term also refers to any specific chemical compound in which one or more atoms have been replaced with one or more different isotopes of the same element.

The entire disclosure of each of the patent documents and scientific articles referred to herein is incorporated by reference in its entirety for all purposes. The function and advantages of these and other embodiments will be more fully understood from the following non-limiting examples. The examples are intended to be illustrative in nature and are not to be considered as limiting the scope of the embodiments discussed herein.

### Examples

There are also several known synthetic routes to provide alkynylbenzenes from the corresponding halobenzenes. For example, reactions in the form of:

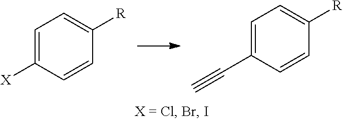

are disclosed in: Khan et al., WO 2017/180923; Miao et al., CN108191711 (2018); Miao et al., CN1030877211 (2013); Wang et al., Liquid Crystals 46(2):257-71 (2019); Li et al., Org. & Biomolec. Chem. 21(29): 5935-38 (2023); Xu, Org. Lett. 16(3):948-51 (2014); Muller et al., Bioconjugate Chem. 28(9):2372-83 (2017).

Compounds of the present disclosure can be made according to known methods published in the art. For example, the following synthetic schemes may be employed to arrive at compounds of the present disclosure:

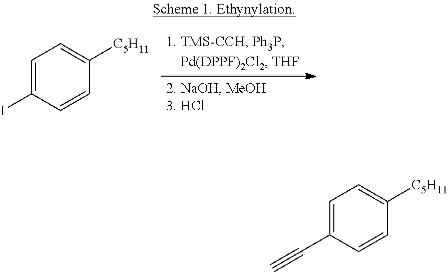

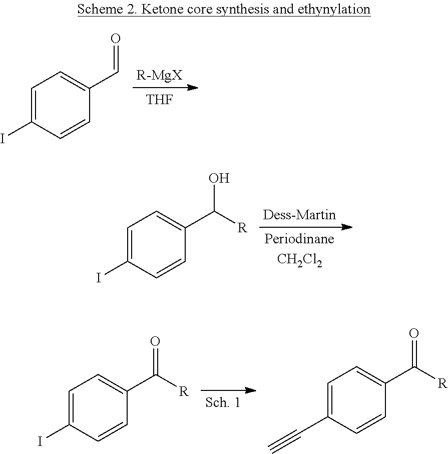

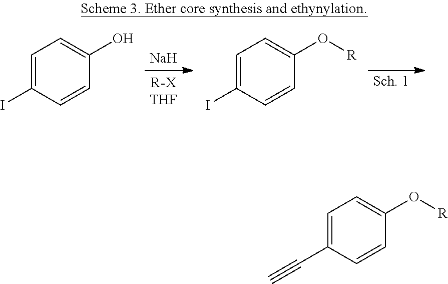

Further elaboration of functional groups within the group R in Schemes 2 and 3, may be carried out according to standard methods, e.g., ester hydrolysis, ketone reduction, alcohol oxidation, ester formation, and terminal alkyne carboxylation.

Ethyl 3-phenylpropiolate is obtained and its olfactory qualities are studied. Both the neat liquid compound and a 10% dilution of the compound in ethanol are examined by an experienced fragrance chemist (a master perfumer). It is found that the compound exhibits a favorable odor profile.

### Synthetic Examples

The Compounds of Examples 1 to 53 may be prepared according to the procedures described hereinbelow. The Compounds of Examples 54 (ethyl 3-phenylpropiolate) and 55 (methyl 3-phenylpropiolate) are commercially available and were purchased on the open market.

¹H-NMR spectra were recorded at 400 MHz on a Bruker Avance AV-I-400 instrument or on a Bruker Avance AV-II-400 instrument. Chemical shift values are expressed in ppm-values relative to tetramethylsilane unless noted otherwise. The following abbreviations or their combinations are used for multiplicity of NMR signals: br=broad, d=doublet, m=multiplet, q=quartet, quint=quintet, s=singlet and t=triplet.

### Purification Method Information:

Method A: Flash chromatography (SiO₂, 0-30% EtOAc in heptanes)
Method B: Flash chromatography (SiO₂, 0-10% EtOAc in heptanes)
Method C: Instrument type: Reveleris™ prep MPLC; Column: Dr. Maisch Reprosil C18 150×25 mm, 10); Flow: 40 mL/min; Column temp: room temperature; Eluent A: 0.1% (v/v) Formic acid in water, Eluent B: 0.1% (v/v) Formic acid in acetonitrile; Gradient: t=0 min 5% B, t=1 min 5% B, t=2 min 30% B, t=17 min 70% B, t=18 min 100% B, t=23 min 100% B;
Detection UV: 220, 254, 340 nm, ELSD.
Method D: Flash chromatography (SiO₂, 0-15% EtOAc in heptanes)
Method E: Flash chromatography (SiO₂, 0-20% Et₂O in heptanes)
Method F: Flash chromatography (SiO₂, 0-20% EtOAc in heptanes)
Method G: Flash chromatography (SiO₂, 0-25% MTBE in heptanes)
Method H: Vacuum distillation (0.1-0.2 mbar, 165° C.)
Method I: Vacuum distillation (0.1-0.2 mbar, 170° C.)
Method J: Vacuum distillation (0.1-0.2 mbar, 180° C.)
Method K: Vacuum distillation (0.1-0.2 mbar, 160° C.)

### LCMS Analytical Method Information:

Method 1: UPLC: Waters I-Class, Acq. Method: UPLC_AN_ACID, Column: XSelect CSH C18 XP (50×2.1 mm 2.5 m), Flow: 0.6 ml/min; Column temp: 40° C., Eluent A: 0.1% formic acid in water, Eluent B: 0.1% formic acid in acetonitrile, Gradient: t=0 min 5% B, t=2.0 min 98% B, t=2.7 min 98% B, Posttime: 0.3 min, Detection PDA: 210-320 nm and 215 nm
Method 2: UPLC: Waters I-Class, Acq. Method: UPLC_AN_BASE, Column: XSelect CSH C18 XP (50×2.1 mm 2.5 m), Flow: 0.6 ml/min; Column temp: 25° C., Eluent A: 10 mM ammonium bicarbonate in water (pH 9.5), Eluent B: acetonitrile, Gradient: t=0 min 5% B, t=2 min 98% B, t=2.7 min 98% B, Posttime: 0.3 min, Detection PDA: 210-320 nm.
Method 3: Acq. Method: U_T3_ACID_00-20, System: Agilent 1290 with SQ-MSD, Column: XSelect HSS T3 XP (50×2.1 mm, 2.5), Flow: 0.8 ml/min; Column temp 40° C., Eluent A: 0.1% Formic acid in Water Eluent B: 0.1% Formic acid in Acetonitrile, Lin. Gradient: t=0 min 0% B, t=4.5 min 20% B, t=5 min 20% B, Posttime: 1 min, Detection: DAD (210, 215, 210-320 nm), Detection: PDA (210-320 nm), Detection: MSD (ESI pos/neg) mass range 90-1500.
Method 4: Acq. Method: U_AN_ACID, System: Agilent 1290 with SQ-MSD, Column: XSelect CSH XP C18 (50×2.1 mm, 2.5), Flow: 0.8 ml/min Column temp 40° C., Eluent A: 0.1% Formic acid in Water, Eluent B: 0.1% Formic acid in Acetonitrile, Lin. Gradient: t=0 min 5% B, t=0.5 min 5% B, t=4.5 min 98% B; t=5 min 98% B, Posttime: 0.5 min, Detection: DAD (210-320 nm, 215 nm), Detection: PDA (210-320 nm), Detection: MSD (ESI pos/neg) mass range 90-1500.
Method 5: Acq. Method: UPLC_AN_BASE, Column: XSelect CSH C18 XP (50×2.1 mm 2.5 μm), Flow: 0.6 ml/min; Column temp: 25° C., Eluent A: 10 mM ammonium bicarbonate in water (pH=9.5), Eluent B: 100% acetonitrile, Gradient: t=0 min 5% B, t=2 min 98% B, t=2.7 min 98% B, Posttime: 0.3 min, Detection PDA: 210-320 nm, Detection ELSD: gas pressure 40 psi, drift tube temp: 50° C.
Method 6: Acq. Method: U_AN_BASE, System: Agilent 1290 with SQ-MSD, Column: XSelect CSH XP C18 (50×2.1 mm, 2.5), Flow: 0.8 ml/min Column temp 25° C., Eluent A: 10 mM Ammonium Bicarbonate in Water (pH 9.5), Eluent B: Acetonitrile, Lin. Gradient: t=0 min 5% B, t=0.5 min 5% B, t=4.5 min 98% B; t=5 min 98% B, Posttime: 0.5 min, Detection: DAD (210-320 nm, 215 nm), Detection: PDA (210-320 nm), Detection: MSD (ESI pos/neg) mass range 90-1500.
Method 7: UPLC: Waters I-Class, Acq. Method: UPLC_AN_ACID, Column: XSelect CSH C18 XP (50×2.1 mm 2.5 μm), Flow: 0.6 ml/min; Column temp: 40° C., Eluent A: 0.1% formic acid in water, Eluent B: 0.1% formic acid in MeCN, Gradient: t=0 min 5% B, t=2 min 98% B, t=2.7 min 98% B, Posttime: 0.3 min, Detection PDA: 210-320 nm, Detection ELSD: gas pressure 40 psi, drift tube temp: 50° C.
Method 8: G40-300_M350, Instrument: Agilent 8890, G7081B 5977B MSD (EI-positive, Det. temp.: 280° C.) Mass range 50-350, Detection FID/Det. temp: 325° C., Column: Agilent DB-5MS (20 m ID 180 m, df 0.18 m), Average velocity: 39 cm/s, Injection vol: 1 μl, Injector temp: 250° C., Split ratio: 100/1, Carrier gas: He; Initial temp: 40° C., Initial time: 0.5 min, Solvent delay: 1.1 min, Rate 120° C./min, 115° C.; 110° C./min, 175° C.; 80° C./min, Final temp 300° C., Hold time 2.0 min.

### Example 1: 3-([1,1′-biphenyl]-4-yl)propiolate

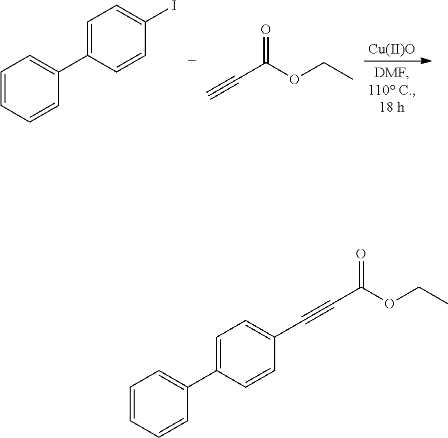

A 2-L 3-neck flask equipped with thermometer was loaded with 4-iodo-1,1′-biphenyl (75.00 g, 1 eq, 267.8 mmol) and DMF (500 mL). Cupric oxide powder (38.34 g, 1.8 Eq, 482.0 mmol) was added, followed by ethyl propiolate (53 mL, 1.9 Eq, 508.7 mmol) and the mixture was heated at 110° C. for 18 h. The mixture was filtered over celite and the filter cake was rinsed with DCM. The filtrate was concentrated, diluted with brine (2 L), and extracted with Et₂O (2×1.5 L). Combined organics were dried over Na₂SO₄, filtered, concentrated, and purified by flash column chromatography (SiO₂, 0-25% EtOAc in heptanes), affording ethyl 3-([1,1′-biphenyl]-4-yl)propiolate (44.638 g, 178.34 mmol, 66.61% yield). LCMS: Method 1, 1.96 min, M+H=251; calcd. 251.297, 1H NMR (400 MHz, CDCl3) δ 7.70-7.54 (m, 6H), 7.51-7.42 (m, 2H), 7.42-7.34 (m, 1H), 4.32 (q, J=7.1 Hz, 2H), 1.37 (t, J=7.1 Hz, 3H).

### Example 2: ethyl 3-(4-(cyclopent-1-en-1-yl)phenyl)propiolate

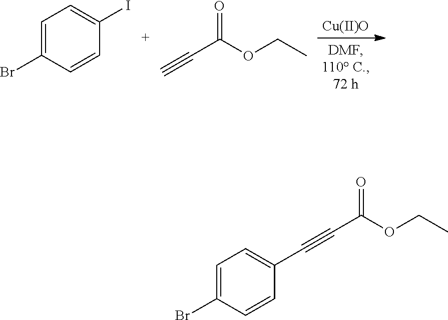

### Step 1: ethyl 3-(4-bromophenyl)propiolate

To 1-bromo-4-iodobenzene (10.0 g, 1 Eq, 35.3 mmol) in DMF (101 mL) were added ethyl propiolate (6.8 mL, 1.9 Eq, 67.2 mmol) and Cupric oxide powder (5.06 g, 802 μL, 1.8 Eq, 63.6 mmol). The mixture was heated at 110° C. for 72 h. The mixture was diluted with Et2O and filtered over celite and the filter cake was rinsed with Et2O. The filtrate was diluted further with Et2O and washed with brine, dried over Na2SO4, filtered and concentrated. Purification by flash column chromatography (SiO2, gradient 0-10% EtO2 in heptanes), afforded ethyl 3-(4-bromophenyl)propiolate (4.52 g, 17 mmol, 47% yield, 94% Purity). GCMS: Method 8, 3.25 min, 251.9; calcd. 251.9.

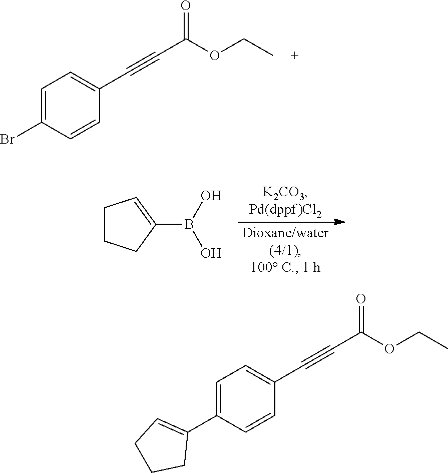 [SMILES: CCOC(=O)C#Cc1ccc(Br)cc1 ; OB(O)C1=CCCC1 ; CCOC(=O)C#Cc1ccc(C2=CCCC2)cc1]

### Step 2: ethyl 3-(4-(cyclopent-1-en-1-yl)phenyl)propiolate

A solution of ethyl 3-(4-bromophenyl)propiolate (2.47 g, 93% Wt., 1 Eq, 9.08 mmol), cyclopent-1-en-1-ylboronic acid (1.12 g, 1.1 Eq, 9.98 mmol) and potassium carbonate (3.76 g, 3 Eq, 27.2 mmol) in 1,4-Dioxane (48.4 mL)/Water (12.1 mL) was flushed with Argon for 5 min. Then 1,1′-Bis(diphenylphosphino)ferrocene-palladium(II) dichloride (332 mg, 0.05 Eq, 454 mol) was added and the mixture was stirred at 100° C. for 1 hour. The mixture was filtered through celite, and the filter cake was rinsed with EtOAc. The organic phase was washed with sat. aq. NaHCO₃, HCl (1M) and brine, dried over Na₂SO₄, filtered and concentrated. Purification by flash chromatography (SiO₂, 0-10% Et₂O in heptanes), followed by purification method C provided ethyl 3-(4-(cyclopent-1-en-1-yl)phenyl)propiolate (568.9 mg, 2.19 mmol, 24.1% yield, 92.5% Purity). LCMS: Method 4, 3.8 min, M+H=241; calcd. 241.302, 1H NMR (400 MHz, CDCl3) δ 7.57-7.48 (m, 2H), 7.46-7.37 (, 2H), 6.30 (p, J 2.3 Hz, 1H), 4.30 (q, J 7.1 Hz, 2H), 2.70 (tq, J 7.0, 2.3 Hz, 2H), 2.55 (tq, J 7.6, 2.5 Hz, 2H), 2.03 (p, J 7.5 Hz, 2H), 1.36 (t, J 7.1 Hz, 3H).

### General Procedure 1: Examples 3-13

The Compounds of Examples 3-13 are made according to the following general procedure via coupling of aryl iodides with propiolate esters using copper(II) oxide catalyst.

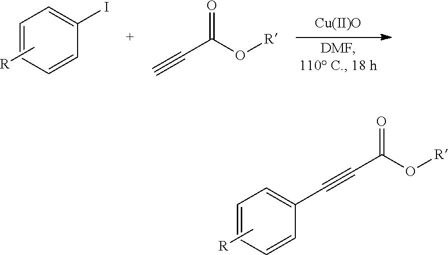

To iodide (1 Eq) in DMF (0.4 M) was added the propiolate (1.9 Eq, R′=Me, Et) and Cu(II) oxide (1.8 Eq) and the mixture was heated at 110° C. for 18 h. The reaction was diluted with Et₂O and filtered over celite and the filter cake was rinsed with Et₂O. The filtrate was diluted further with Et₂O, washed with brine, dried over Na₂SO₄, filtered and concentrated. Purification (see method) provided the target.

The following examples were prepared according to General Procedure 1, starting from their corresponding iodide and propiolate.

| | Purif. | | | |
|---|---|---|---|---|
| Ex. | Method | Yield | LCMS | NMR |
| 3 | D | 1235.4 mg | Method 2, | 1H NMR (400 MHz, CDCl3) δ 7.66 (d, J = |
| | | (59% yield, | 1.91 min, | 8.6 Hz, 2H), 7.63-7.57 (m, 4H), 7.46 (dd, |
| | | 98.97% purity) | M + H = 237; | J = 8.3, 6.8 Hz, 2H), 7.42-7.35 (m, 1H), |
| | | | calcd. 237.27 | 3.86 (s, 3H). |
| 4 | D | 898.6 mg | Method 7, | 1H NMR (400 MHz, CDCl3) δ 7.43 (d, J = |
| | | (44.3% yield, | 1.73 min, | 7.9 Hz, 1H), 7.06 (s, 1H), 6.99 (d, J = 8.7 Hz, |
| | | 99.95% purity) | M + H = 189; | 1H), 3.83 (s, 3H), 2.45 (s, 3H), 2.34 (s, 3H). |
| | | | calcd. 189.226 | |
| 5 | D | 1250.4 mg | Method 7, | 1H NMR (400 MHz, CDCl3) δ 7.43 (d, J = |
| | | (57.4% yield, | 1.83 min, | 7.8 Hz, 1H), 7.05 (s, 1H), 6.99 (d, J = 7.8 Hz, |
| | | 99.27% purity) | M + H = 203; | 1H), 4.29 (q, J = 7.2 Hz, 2H), 2.45 (s, 3H), |
| | | | calcd. 203.253 | 2.34 (s, 3H), 1.36 (t, J = 7.1 Hz, 3H). |
| 6 | F | 954 mg | Method 1, | 1H NMR (400 MHz, CDCl3) δ 7.55-7.48 |
| | | (46.4% yield, | 1.84 min, | (m, 2H), 7.26-7.17 (m, 2H), 3.84 (s, 3H), |
| | | 99.54% purity) | M + H = 203; | 2.93 (hept, J = 7.0 Hz, 1H), 1.25 (d, J = 7.0 |
| | | | calcd. 203.253 | Hz, 6H). |
| 7 | F | 1460.5 mg | Method 1, | 1H NMR (400 MHz, CDCl3) δ 7.55-7.48 |
| | | (66.5% yield, | 1.92 min, | (m, 2H), 7.25-7.19 (m, 2H), 4.29 (q, J = 7.1 |
| | | 99.65% purity) | M + H = 217; | Hz, 2H), 2.92 (hept, J = 7.0 Hz, 1H), 1.36 (t, |
| | | | calcd. 217.28 | J = 7.1 Hz, 3H), 1.25 (d, J = 6.9 Hz, 6H). |
| 8 | F | 1143.9 mg | Method 1, | 1H NMR (400 MHz, CDCl3) δ 8.33 (dd, J = |
| | | (55.3% yield, | 1.74 min, | 8.3, 1.1 Hz, 1H), 7.96 (dt, J = 8.5, 1.1 Hz, |
| | | 99.86% purity) | M + H = 211; | 1H), 7.87 (ddd, J = 10.7, 7.2, 1.1 Hz, 2H), |
| | | | calcd. 211.232 | 7.63 (ddd, J = 8.3, 6.9, 1.4 Hz, 1H), 7.57 (ddd, |
| | | | | J = 8.0, 6.8, 1.3 Hz, 1H), 7.47 (dd, J = 8.3, 7.2 |
| | | | | Hz, 1H), 3.90 (s, 3H). |
| 9 | F | 1257.3 mg | Method 1, | 1H NMR (400 MHz, CDCl3) δ 8.38-8.31 (m, |
| | | (57% yield, | 1.84 min, | 1H), 7.95 (dt, J = 8.3, 1.0 Hz, 1H), 7.87 (ddd, J = |
| | | 99.53% purity) | M + H = 225; | 10.3, 7.2, 1.1 Hz, 2H), 7.63 (ddd, J = 8.4, 6.9, |
| | | | calcd. 225.259 | 1.4 Hz, 1H), 7.56 (ddd, J = 8.2, 6.8, 1.3 Hz, |
| | | | | 1H), 7.47 (dd, J = 8.3, 7.2 Hz, 1H), 4.36 (q, |
| | | | | J = 7.2 Hz, 2H), 1.40 (t, J = 7.1 Hz, 3H). |
| 10 | F | 1277.8 mg | Method 7, | 1H NMR (400 MHz, CDCl3) δ 6.89 (s, 2H), |
| | | (62.2% yield, | 1.85 min, | 3.84 (s, 3H), 2.44 (s, 6H), 2.30 (s, 3H). |
| | | 99.69% purity) | M + H = 203; | |
| | | | calcd. 203.253 | |
| 11 | F | 1390.9 mg | Method 7, | 1H NMR (400 MHz, CDCl3) δ 6.88 (s, 2H), |
| | | (63.3% yield, | 1.94 min, | 4.30 (q, J = 7.2 Hz, 2H), 2.44 (s, 6H), 2.29 (s, |
| | | 99.81% purity) | M + H = 217; | 3H), 1.36 (t, J = 7.1 Hz, 3H). |
| | | | calcd. 217.28 | |
| 12 | F | 928 mg | Method 7, | 1H NMR (400 MHz, CDCl3) δ 7.54-7.46 |
| | | (66.5% yield, | 2.08 min, | (m, 2H), 7.24-7.16 (m, 2H), 3.83 (s, 3H), |
| | | 97.5% purity) | M + H = 243.2; | 2.58-2.46 (m, 1H), 1.92-1.79 (m, 4H), 1.79- |
| | | | calcd. 243.318 | 1.71 (m, 1H), 1.48-1.31 (m, 4H), 1.31- |
| | | | | 1.18 (m, 1H). |
| 13 | F | 841.1 mg | Method 7, | 1H NMR (400 MHz, CDCl3) δ 7.54-7.46 |
| | | (57% yield, | 2.15 min, | (m, 2H), 7.24-7.15 (m, 2H), 4.29 (q, J = 7.1 |
| | | 98.84% purity) | M + H = 257.1; | Hz, 2H), 2.59-2.45 (m, 1H), 1.92-1.80 (m, |
| | | | calcd. 257.345 | 4H), 1.80-1.71 (m, 1H), 1.47-1.31 (m, |
| | | | | 7H), 1.31-1.17 (m, 1H). |

### General Procedure 2: Examples 14-35

The Compounds of Examples 14-35 are made according to the following general procedure via, coupling of phenylacetylenes with alkyl chloroformates.

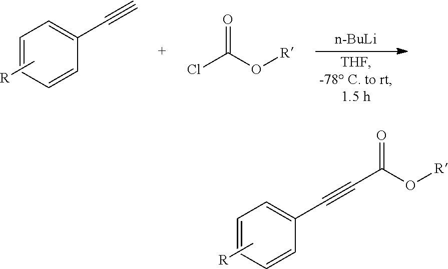

To acetylene (1 Eq) in dry THF (0.5 M) at −78° C. was added n-BuLi, 1.6M in hexanes (1.6 molar, 1.1 Eq) dropwise and the mixture was stirred for 30 min at −78° C. The cooling bath was removed, chloroformnate (1 Eq, R′=Me, Et) was added and the mixture was allowed to warm to room temperature and stirred for 1 h. The reaction was concentrated and purification (see method) provided the target.

The following examples were prepared according to General Procedure 2, starting from their corresponding acetylene and chloroformate.

| | Purif. | | | |
|---|---|---|---|---|
| Ex. | Method | Yield | LCMS | NMR |
| 14 | A | 1232.5 mg | Method 4, | 1H NMR (400 MHz, CDCl3) δ 7.58-7.50 (m, 2H), |
| | | (80% yield, | 2.98 min, | 6.92-6.80 (m, 2H), 4.29 (q, J = 7.2 Hz, 2H), 3.84 |
| | | 99.13% purity) | M + H = 205; | (s, 3H), 1.35 (t, J = 7.1 Hz, 3H). |
| | | | calcd. 205.225 | |
| 15 | A | 1267.2 mg | Method 1, | 1H NMR (400 MHz, CDCl3) δ 7.58-7.50 (m, 2H), |
| | | (88% yield, | 1.51 min, | 6.93-6.85 (m, 2H), 3.83 (d, J = 3.5 Hz, 6H), 2.01 |
| | | 97.33% purity) | M + H = 191.04; | (s, 1H). |
| | | | calcd. 191.198 | |
| 16 | D | 1098.6 mg | Method 1, | 1H NMR (400 MHz, CDCl3) δ 7.56-7.49 (m, 2H), |
| | | (78.6% yield, | 1.64 min, | 6.90-6.83 (m, 2H), 4.06 (q, J = 7.0 Hz, 2H), 3.83 |
| | | 99.31% purity) | M + H = 205; | (s, 3H), 1.43 (t, J = 7.0 Hz, 3H). |
| | | | calcd. 205.225 | |
| 17 | D | 1227.9 mg | Method 1, | 1H NMR (400 MHz, CDCl3) δ 7.57-7.48 (m, 2H), |
| | | (82.2% yield, | 1.74 min, | 6.91-6.79 (m, 2H), 4.29 (q, J = 7.1 Hz, 2H), 4.06 |
| | | 99.65% purity) | M + H = 219; | (q, J = 7.0 Hz, 2H), 1.43 (t, J = 7.0 Hz, 3H), 1.35 (t, |
| | | | calcd. 219.252 | J = 7.2 Hz, 3H). |
| 18 | D | 1039 mg | Method 1, | 1H NMR (400 MHz, CDCl3) δ 8.16 (d, J = 1.6 Hz, |
| | | (75.2% yield, | 1.74 min, | 1H), 7.84 (dd, J = 7.8, 3.4 Hz, 3H), 7.61-7.50 (m, |
| | | 98.22% purity) | M + H = 211; | 3H), 3.87 (s, 3H). |
| | | | calcd. 211.232 | |
| 19 | D | 1127.8 mg | Method 1, | 1H NMR (400 MHz, CDCl3) δ 8.16 (d, J = 1.6 Hz, |
| | | (76.5% yield, | 1.84 min, | 1H), 7.88-7.80 (m, 3H), 7.61-7.50 (m, 3H), 4.33 |
| | | 96.11% purity) | M + H = 225; | (q, J = 7.1 Hz, 2H), 1.38 (t, J = 7.1 Hz, 3H). |
| | | | calcd. 225.259 | |
| 20 | B | 1216 mg | Method 4, | 1H NMR (400 MHz, CDCl3) δ 7.54 (dd, J = 7.7, 1.4 |
| | | (75% yield, | 3.18 min, | Hz, 1H), 7.33 (td, J = 7.6, 1.4 Hz, 1H), 7.24 (d, J = |
| | | 98.72% purity) | M + H = 189; | 7.7 Hz, 1H), 7.18 (t, J = 7.7 Hz, 1H), 4.30 (q, J = |
| | | | calcd. 189.226 | 7.1 Hz, 2H), 2.50 (s, 3H), 1.36 (t, J = 7.1 Hz, 3H). |
| 21 | B | 629.6 mg | Method 4, | 1H NMR (400 MHz, CDCl3) δ 7.54 (dd, J = 7.8, |
| | | (42% yield, | 2.97 min, | 1.4 Hz, 1H), 7.34 (td, J = 7.6, 1.4 Hz, 1H), 7.24 (d, |
| | | 97.79% purity) | M + H = 175; | J = 7.6 Hz, 1H), 7.19 (t, J = 7.3 Hz, 1H), 3.84 (s, |
| | | | calcd. 175.199 | 3H), 2.49 (s, 3H). |
| 22 | B | 627.1 mg | Method 4, | 1H NMR (400 MHz, CDCl3) δ 7.53 (dd, J = 7.6, |
| | | (44% yield, | 2.7 min, | 1.7 Hz, 1H), 7.41 (ddd, J = 8.8, 7.5, 1.8 Hz, 1H), |
| | | 98.48% purity) | M + H = 191; | 6.94 (t, J = 7.5 Hz, 1H), 6.91 (d, J = 8.4 Hz, 1H), |
| | | | calcd. 191.198 | 3.90 (s, 3H), 3.84 (s, 3H). |
| 23 | B | 1178.5 mg | Method 4, | 1H NMR (400 MHz, CDCl3) δ 7.53 (dd, J = 7.6, |
| | | (76% yield, | 2.92 min, | 1.7 Hz, 1H), 7.41 (ddd, J = 8.7, 7.4, 1.7 Hz, 1H), |
| | | 98.02% purity) | M + H = 205; | 6.94 (t, J = 7.5 Hz, 1H), 6.90 (d, J = 8.3 Hz, 1H), |
| | | | calcd. 205.225 | 4.30 (q, J = 7.1 Hz, 2H), 3.90 (s, 3H), 1.35 (t, J = |
| | | | | 7.1 Hz, 3H). |
| 24 | E | 945.4 mg | Method 1, | 1H NMR (400 MHz, CDCl3) δ 7.56-7.50 (m, 2H), |
| | | (69% yield, | 1.91 min, | 7.44-7.35 (m, 2H), 3.84 (s, 3H), 1.32 (s, 9H). |
| | | 96.64% purity) | M + H = 217.03; | |
| | | | calcd. 217.28 | |
| 25 | E | 1312.2 mg | Method 1, | 1H NMR (400 MHz, CDCl3) δ 7.55-7.50 (m, 2H), |
| | | (90% yield, | 1.99 min, | 7.42-7.35 (m, 2H), 4.30 (q, J = 7.2 Hz, 2H), 1.36 |
| | | 98.85% purity) | M + H = 231.07; | (t, J = 7.1 Hz, 3H), 1.32 (s, 9H). |
| | | | calcd. 231.307 | |
| 26 | E | 1085.8 mg | Method 4, | 1H NMR (400 MHz, CDCl3) δ 7.53-7.44 (m, 2H), |
| | | (72% yield, | 2.96 min, | 7.22-7.13 (m, 2H), 3.84 (s, 3H), 2.38 (s, 3H). |
| | | 98.24% purity) | M + H = 175; | |
| | | | calcd. 175.199 | |
| 27 | E | 1058.2 mg | Method 4, | 1H NMR (400 MHz, CDCl3) δ 7.55-7.44 (m, 2H), |
| | | (66% yield, | 3.17 min, | 7.18 (d, J = 7.9 Hz, 2H), 4.29 (q, J = 7.1 Hz, 2H), |
| | | 98.25% purity) | M + H = 189; | 2.38 (s, 3H), 1.35 (t, J = 7.1 Hz, 3H). |
| | | | calcd. 189.226 | |
| 28 | E | 881.3 mg | Method 4, | 1H NMR (400 MHz, CDCl3) δ 7.55-7.45 (m, 2H), |
| | | (62% yield, | 3.19 min, | 7.23-7.15 (m, 2H), 3.84 (s, 3H), 2.67 (q, J = 7.6 |
| | | 96.88% purity) | M + H = 189; | Hz, 2H), 1.24 (t, J = 7.6 Hz, 3H). |
| | | | calcd. 189.226 | |
| 29 | E | 1390 mg | Method 4, | 1H NMR (400 MHz, CDCl3) δ 7.54-7.42 (m, 2H), |
| | | (90% yield, | 3.38 min, | 7.23-7.16 (m, 2H), 4.29 (q, J = 7.1 Hz, 2H), 2.67 |
| | | 97.72% purity) | M + H = 203; | (q, J = 7.6 Hz, 2H), 1.36 (t, J = 7.2 Hz, 3H), 1.24 (t, |
| | | | calcd. 203.253 | J = 7.6 Hz, 3H). |
| 30 | E | 1166 mg | Method 4, | 1H NMR (400 MHz, CDCl3) δ 7.29 (t, J = 7.8 Hz, |
| | | (82% yield, | 2.8 min, | 1H), 7.18 (dt, J = 7.5, 1.2 Hz, 1H), 7.10 (dd, J = |
| | | 98.24% purity) | M + H = 191; | 2.6, 1.4 Hz, 1H), 7.00 (ddd, J = 8.3, 2.7, 1.1 Hz, |
| | | | calcd. 191.198 | 1H), 3.84 (s, 3H), 3.81 (s, 3H). |
| 31 | E | 1342.2 mg | Method 4, | 1H NMR (400 MHz, CDCl3) δ 7.28 (t, J = 7.6 Hz, |
| | | (87% yield, | 3.01 min, | 2H), 7.19 (dt, J = 7.7, 1.3 Hz, 1H), 7.10 (dd, J = |
| | | 98.45% purity) | M + H = 205; | 2.7, 1.5 Hz, 1H), 7.00 (ddd, J = 8.3, 2.7, 1.0 Hz, |
| | | | calcd. 205.225 | 1H), 4.30 (q, J = 7.2 Hz, 2H), 3.81 (s, 3H), 1.36 (t, |
| | | | | J = 7.1 Hz, 3H). |
| 32 | G | 769.9 mg | Method 7, | 1H NMR (400 MHz, CDCl3) δ 7.66-7.51 (m, |
| | | (43% yield, | 1.73 min, | 8H), 7.49-7.41 (m, 4H), 7.41-7.30 (m, 8H), 4.20 |
| | | 100% purity) | M + H = 188.96; | (t, J = 6.7 Hz, 8H), 1.75 (h, J = 7.2 Hz, 8H), 1.00 (t, |
| | | | calcd. 189.226 | J = 7.4 Hz, 12H). |
| 33 | E + C | 1189.3 mg | Method 7, | 1H NMR (400 MHz, CDCl3) δ 7.67-7.53 (m, 2H), |
| | | (66% yield, | 1.66 min, | 7.53-7.42 (m, 1H), 7.42-7.30 (m, 2H), 5.98 (ddt, |
| | | 99.02% purity) | M + H = 186.98; | J = 16.4, 10.4, 5.9 Hz, 1H), 5.41 (dq, J = 17.1, 1.5 |
| | | | calcd. 187.21 | Hz, 1H), 5.32 (dq, J = 10.2, 1.2 Hz, 1H), 4.73 (dt, |
| | | | | J = 6.0, 1.4 Hz, 2H). |
| 34 | C | 441.6 mg | Method 7, | 1H NMR (400 MHz, CDCl3) δ 7.56-7.49 (m, 2H), |
| | | (67% yield, | 1.97 min, | 7.25-7.18 (m, 2H), 6.25 (s, 1H), 4.30 (q, J = 7.1 |
| | | 99.82% purity) | M + H = 229.07; | Hz, 2H), 1.92 (d, J = 1.4 Hz, 3H), 1.88 (d, J = 1.4 |
| | | | calcd. 229.291 | Hz, 3H), 1.36 (t, J = 7.1 Hz, 3H). |
| 35 | C | 784.2 mg | Method 4, | 1H NMR (400 MHz, CDCl3) δ 7.59-7.49 (m, |
| | | (70% yield, | 3.47 min, | 2H), 7.25-7.17 (m, 2H), 6.29-6.18 (m, 1H), 3.84 |
| | | 99.35% purity) | M + H = 215; | (s, 3H), 1.90 (dd, J = 18.8, 1.5 Hz, 6H). |
| | | | calcd. 215.264 | |

### General Procedure 3: Examples 36-53

The Compounds of Examples 36-53 are made according to the following general procedure via coupling of dibromostyrenes with alkyl chloroformates using n-butyl lithium.

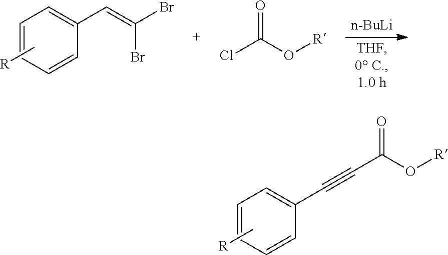

To dibromostyrene (1 Eq) in dry THF (0.15 M) at 0° C. was added n-BuLi, 1.6M in hexanes (3 Eq) dropwise and the mixture was stirred for 30 min at 0° C. Chloroformate (1 Eq, R′-Me, Et) was added and the mixture was stirred for 30 min at 0° C. The reaction was quenched by addition of sat. aq. NH₄Cl and partially concentrated to remove the THF. Diluted with water and DCM and layers separated. The organic phase was washed with brine, dried over Na₂SO₄, filtered and concentrated. Purification (see method) provided the target.

Representative dibromostyrene synthetic procedures are provided below.

The following examples were prepared according to General Procedure 3, starting from their corresponding dibromostyrene and chloroformate.

| | Purif. | | | |
|---|---|---|---|---|
| Ex. | Method | Yield | LCMS | NMR |
| 36 | C | 487.4 mg | Method 4, | 1H NMR (400 MHz, CDCl3) δ 7.41 (d, J = 2.2 Hz, |
| | | (32.3% yield, | 3 min, | 1H), 7.39 (dd, J = 4.6, 1.4 Hz, 1H), 7.27 (s, 1H), |
| | | 99.12% purity) | M + H = 317; | 7.25 (d, J = 0.9 Hz, 1H), 3.84 (s, 3H), 2.35 (s, 3H). |
| | | | calcd. 175.199 | |
| 37 | B | 1007.3 mg | Method 4, | 1H NMR (400 MHz, CDCl3) δ 7.41 (d, J = 2.6 Hz, |
| | | (61.8% yield, | 3.23 min, | 1H), 7.39 (d, J = 4.7 Hz, 1H), 7.25 (d, J = 1.2 Hz, |
| | | 98.65% purity) | M + H = 189; | 1H), 4.30 (q, J = 7.1 Hz, 2H), 2.35 (s, 3H), 1.36 (t, |
| | | | calcd. 189.226 | J = 7.1 Hz, 3H). 1H fully under CDCl3. |
| 38 | E | 784.1 mg | Method 4, | 1H NMR (400 MHz, CDCl3) δ 7.34-7.27 (m, 2H), |
| | | (92% yield, | 3.48 min, | 7.05 (d, J = 7.8 Hz, 1H), 3.83 (s, 3H), 2.84-2.68 |
| | | 96.9% purity) | M + H = 215; | (m, 4H), 1.86-1.70 (m, 4H). |
| | | | calcd. 215.264 | |
| 39 | E | 803.4 mg | Method 4, | 1H NMR (400 MHz, CDCl3) δ 7.33-7.27 (m, 2H), |
| | | (88% yield, | 3.66 min, | 7.05 (d, J = 7.7 Hz, 1H), 4.29 (q, J = 7.2 Hz, 2H), |
| | | 99.33% purity) | M + H = 229; | 2.83-2.68 (m, 4H), 1.85-1.73 (m, 4H), 1.35 (t, J = |
| | | | calcd. 229.291 | 7.1 Hz, 3H). |
| 40 | E | 719 mg | Method 7, | 1H NMR (400 MHz, CDCl3) δ 7.44 (s, 1H), 7.37 |
| | | (72.3% yield, | 1.78 min, | (dd, J = 7.8, 1.4 Hz, 1H), 7.22 (d, J = 7.8 Hz, 1H), |
| | | 98.85% purity) | M + H = 201; | 3.83 (s, 3H), 2.92 (q, J = 7.9 Hz, 4H), 2.09 (p, J = |
| | | | calcd. 201.237 | 7.4 Hz, 2H). |
| 41 | E | 673.4 mg | Method 7, | 1H NMR (400 MHz, CDCl3) δ 7.44 (s, 1H), 7.37 |
| | | (63.3% yield, | 1.88 min, | (dd, J = 7.8, 1.4 Hz, 1H), 7.21 (d, J = 7.7 Hz, 1H), |
| | | 99.35% purity) | M + H = 215; | 4.29 (q, J = 7.1 Hz, 2H), 2.91 (q, J = 7.8 Hz, 4H), |
| | | | calcd. 215.264 | 2.09 (p, J = 7.5 Hz, 2H), 1.35 (t, J = 7.1 Hz, 3H). |
| 42 | E | 593.9 mg | Method 4, | 1H NMR (400 MHz, CDCl3) δ 7.53-7.46 (m, 2H), |
| | | (79.1% yield, | 3.29 min, | 7.01-6.93 (m, 2H), 3.83 (s, 3H), 1.39 (s, 9H). |
| | | 99.46% purity) | M + H = 177; | |
| | | | calcd. 233.279 | |
| 43 | E | 774.7 mg | Method 4, | 1H NMR (400 MHz, CDCl3) δ 7.54-7.42 (m, 2H), |
| | | (97.6% yield, | 3.47 min, | 7.03-6.89 (m, 2H), 4.29 (q, J = 7.2 Hz, 2H), 1.39 |
| | | 98.87% purity) | M + H = 247; | (s, 9H), 1.35 (t, J = 7.1 Hz, 3H). |
| | | | calcd. 247.306 | |
| 44 | E | 747.6 mg | Method 4, | 1H NMR (400 MHz, CDCl3) δ 7.53-7.46 (m, 2H), |
| | | (62% yield, | 3.61 min, | 7.20-7.07 (m, 2H), 3.84 (s, 3H), 2.49 (d, J = 7.2 |
| | | 97.87% purity) | M + H = 217; | Hz, 2H), 1.93-1.79 (m, 1H), 0.90 (d, J = 6.6 Hz, 6H). |
| | | | calcd. 217.28 | |
| 45 | E | 517.4 mg | Method 4, | 1H NMR (400 MHz, CDCl3) δ 7.53-7.46 (m, 2H), |
| | | (40% yield, | 3.78 min, | 7.18-7.09 (m, 2H), 4.29 (q, J = 7.2 Hz, 2H), 2.49 |
| | | 98.17% purity) | M + H = 231; | (d, J = 7.2 Hz, 2H), 1.95-1.79 (m, 1H), 1.36 (t, J = |
| | | | calcd. 231.307 | 7.1 Hz, 3H), 0.90 (d, J = 6.6 Hz, 6H). |
| 46 | E | 1248.2 mg | Method 4, | 1H NMR (400 MHz, CDCl3) δ 7.55-7.45 (m, 2H), |
| | | (92% yield, | 3.2 min, | 6.89-6.79 (m, 2H), 4.59 (p, J = 6.1 Hz, 1H), 3.83 |
| | | 99.8% purity) | M + H = 219; | (s, 3H), 1.35 (d, J = 6.1 Hz, 6H). |
| | | | calcd. 219.252 | |
| 47 | E | 1333.1 mg | Method 4, | 1H NMR (400 MHz, CDCl3) δ 7.55-7.45 (m, 2H), |
| | | (92% yield, | 3.38 min, | 6.89-6.81 (m, 2H), 4.59 (hept, J = 6.0 Hz, 1H), |
| | | 99.78% purity) | M + H = 233; | 4.29 (q, J = 7.1 Hz, 2H), 1.39-1.29 (m, 9H). |
| | | | calcd. 233.279 | |
| 48 | G + H | 624.8 mg | Method 5, | 1H NMR (400 MHz, CDCl3) δ 7.55-7.47 (m, 2H), |
| | | (44.9% yield, | 1.9 min, | 6.89-6.81 (m, 2H), 4.34 (h, J = 6.0 Hz, 1H), 3.83 |
| | | 98.59% purity) | M + H = 233.2; | (s, 3H), 1.82-1.57 (m, 2H), 1.30 (d, J = 6.0 Hz, |
| | | | calcd. 233.279 | 3H), 0.97 (t, J = 7.5 Hz, 3H). |
| 49 | G + H | 746.7 mg | Method 5, | 1H NMR (400 MHz, CDCl3) δ 7.61-7.42 (m, 2H), |
| | | (50.6% yield, | 1.99 min, | 6.94-6.76 (m, 2H), 4.35 (h, J = 6.0 Hz, 1H), 4.29 |
| | | 99.03% purity) | M + H = 247.2; | (q, J = 7.1 Hz, 2H), 1.83-1.57 (m, 2H), 1.35 (t, J = |
| | | | calcd. 247.306 | 7.1 Hz, 3H), 1.30 (d, J = 6.1 Hz, 3H), 0.97 (t, J = 7.5 |
| | | | | Hz, 3H). |
| 50 | G + I | 821.9 mg | Method 5, | 1H NMR (400 MHz, CDCl3) δ 7.60-7.43 (m, 2H), |
| | | (52.7% yield, | 2.08 min, | 6.93-6.81 (m, 2H), 4.34 (h, J = 6.1 Hz, 1H), 4.18 |
| | | 97.78% purity) | M + H = 261.2; | (t, J = 6.8 Hz, 2H), 1.83-1.57 (m, 4H), 1.30 (d, J = |
| | | | calcd. 261.333 | 6.1 Hz, 3H), 1.00 (t, J = 7.6 Hz, 3H), 0.97 (t, J = 7.5 |
| | | | | Hz, 3H). |
| 51 | G + J | 544.4 mg | Method 5, | 1H NMR (400 MHz, CDCl3) δ 7.57-7.43 (m, 2H), |
| | | (35.2% yield, | 2.02 min, | 6.91-6.77 (m, 2H), 5.98 (ddt, J = 16.3, 10.4, 5.9 |
| | | 96.16% purity) | M + H = 259.1; | Hz, 1H), 5.40 (dq, J = 17.2, 1.5 Hz, 1H), 5.30 (dq, |
| | | | calcd. 259.317 | J = 10.5, 1.2 Hz, 1H), 4.72 (dt, J = 5.8, 1.4 Hz, 2H), |
| | | | | 4.34 (h, J = 6.1 Hz, 1H), 1.83-1.57 (m, 2H), 1.30 |
| | | | | (d, J = 6.0 Hz, 3H), 0.97 (t, J = 7.4 Hz, 3H). |
| 52 | G + K | 565.9 mg | Method 5, | 1H NMR (400 MHz, CDCl3) δ 7.39 (dd, J = 7.4, 1.5 |
| | | (48.2% yield, | 1.93 min, | Hz, 1H), 7.15 (dd, J = 7.6, 1.4 Hz, 1H), 7.09 (t, J = |
| | | 97.08% purity) | M + H = 215.1; | 7.6 Hz, 1H), 3.84 (s, 3H), 2.92 (t, J = 6.2 Hz, 2H), |
| | | | calcd. 215.264 | 2.76 (t, J = 6.0 Hz, 2H), 1.88-1.73 (m, 4H). |
| 53 | G + K | 486.1 mg | Method 5, | 1H NMR (400 MHz, CDCl3) δ 7.39 (dd, J = 7.3, 1.4 |
| | | (38.9% yield, | 2.03 min, | Hz, 1H), 7.15 (dd, J = 7.6, 1.4 Hz, 1H), 7.08 (t, J = |
| | | 97.19% purity) | M + H = 229.2; | 7.6 Hz, 1H), 4.30 (q, J = 7.1 Hz, 2H), 2.92 (t, J = 6.2 |
| | | | calcd. 229.291 | Hz, 2H), 2.76 (t, J = 6.1 Hz, 2H), 1.88-1.73 (m, |
| | | | | 4H), 1.36 (t, J = 7.1 Hz, 3H). |

### Synthetic Intermediates

The following procedures provide synthetic intermediates used in General Procedures 1, 2, or 3 shown above.

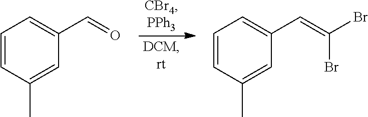

### 1-(2,2-dibromovinyl)-3-methylbenzene

To a solution of carbon tetrabromide (8.28 g, 1.5 Eq, 25.0 mmol) and triphenylphosphine (13.1 g, 3 Eq, 49.9 mmol) in Dichloromethane (33.3 mL) at room temperature was added m-formyltoluene (1.96 mL, 1 Eq, 16.6 mmol) and the mixture was stirred for 30 min at room temperature. Water was added and the mixture was extracted with DCM (3×). The organic phase was washed with brine, dried over Na₂SO₄, filtered and concentrated. Purification by flash column chromatography (SiO₂, 0-10% EtOAc in heptanes) afforded 1-(2,2-dibromovinyl)-3-methylbenzene (4.78 g, 17.1 mmol, 100% yield, 98.5% Purity). 1H NMR (400 MHz, CDCl3) δ 7.45 (s, 1H), 7.38-7.29 (m, 2H), 7.28-7.23 (m, 1H), 7.16 (s, 1H), 2.37 (s, 3H).

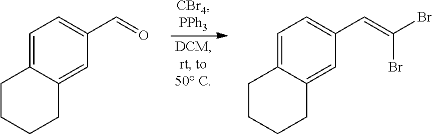

### 6-(2,2-dibromovinyl)-1,2,3,4-tetrahydronaphthalene

To a solution of carbon tetrabromide (9.31 g, 1.61 Eq, 28.1 mmol) and triphenylphosphine (14.7 g, 3.22 Eq, 56.0 mmol) in Dichloromethane (174 mL) at room temperature was added 5,6,7,8-tetrahydronaphthalene-2-carbaldehyde (3.00 g, 93% Wt., 1 Eq, 17.4 mmol) and the mixture was stirred for 1 h at room temperature and then for 1 h at 50° C. Water was added and the mixture was extracted with DCM (3×). The organic phase was washed with brine, dried over Na₂SO₄, filtered and concentrated. Purification by flash column chromatography (SiO₂, 0-10% EtOAc in heptanes) afforded 6-(2,2-dibromovinyl)-1,2,3,4-tetrahydronaphthalene (3.58 g, 8.5 mmol, 49% yield, 75% Purity). 1H NMR (400 MHz, CDCl3) δ 7.41 (s, 1H), 7.29 (ddd, J=8.1, 3.9, 2.1 Hz, 1H), 7.23 (dd, J=11.4, 2.0 Hz, 1H), 7.05 (d, J=8.0 Hz, 1H), 2.82-2.68 (m, 4H), 1.79 (p, J=3.2 Hz, 4H).

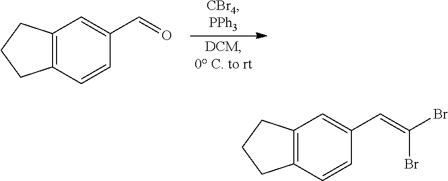

### 5-(2,2-dibromovinyl)-2,3-dihydro-1H-indene

To a solution of carbon tetrabromide (9.26 g, 1.5 Eq, 27.9 mmol) and triphenylphosphine (14.6 g, 3 Eq, 55.8 mmol) in Dichloromethane (75 mL) at 0° C. was added 2,3-dihydro-1H-indene-5-carbaldehyde (2.72 g, 1 Eq, 18.6 mmol) in Dichloromethane (5 mL) and the mixture was stirred for 1 h at room temperature. Water was added and the mixture was extracted with DCM. The organic phase was washed with brine, dried over Na₂SO₄, filtered and concentrated. Purification by flash column chromatography (SiO₂, 0-15% EtOAc in heptanes) afforded 5-(2,2-dibromovinyl)-2,3-dihydro-1H-indene (3.03 g, 10.0 mmol, 53.9% yield). GCMS: Method 8, 3.47 min, 301.9; calcd. 302.1.

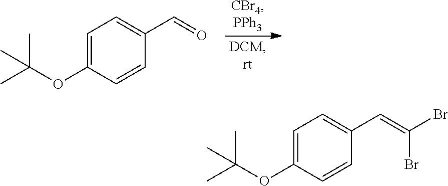

### 1-(tert-butoxy)-4-(2,2-dibromovinyl)benzene

To a solution of carbon tetrabromide (9.31 g, 1.67 Eq, 28.1 mmol) and triphenylphosphine (14.7 g, 3.33 Eq, 56.0 mmol) in Dichloromethane (168 mL) at room temperature was added 4-(tert-butoxy)benzaldehyde (2.93 mL, 1 Eq, 16.8 mmol) and the mixture was stirred for 1 h at room temperature. Water was added and the mixture was extracted with DCM (3×). The organic phase was washed with brine, dried over Na₂SO₄, filtered and concentrated. Purification by flash column chromatography (SiO₂, 0-10% EtOAc in heptanes) afforded 1-(tert-butoxy)-4-(2,2-dibromovinyl)benzene (2.17 g, 6.50 mmol, 38.6% yield). Used as such without further analysis.

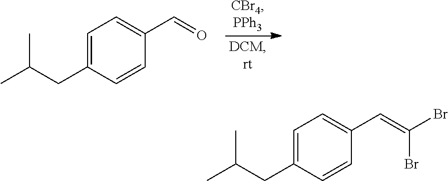

### 1-(2,2-dibromovinyl)-4-isobutylbenzene

To a solution of carbon tetrabromide (9.2 g, 1.5 Eq, 28 mmol) and triphenylphosphine (15 g, 3 Eq, 55 mmol) in Dichloromethane (180 mL) at room temperature was added 4-isobutylbenzaldehyde (3.1 mL, 1 Eq, 18 mmol) and the mixture was stirred for 1 h at room temperature. Water was added and the mixture was extracted with DCM (3×). The organic phase was washed with brine, dried over Na₂SO₄, filtered and concentrated. Purification by flash column chromatography (SiO₂, 0-10% EtOAc in heptanes) afforded 1-(2,2-dibromovinyl)-4-isobutylbenzene (3.6 g, 11 mmol, 61% yield). 1H NMR (400 MHz, CDCl3) δ 7.53-7.38 (m, 3H), 7.14 (dd, J=8.0, 1.4 Hz, 2H), 2.48 (dd, J=9.7, 7.1 Hz, 2H), 1.87 (dpd, J=13.6, 6.7, 1.4 Hz, 1H), 0.90 (d, J=6.7 Hz, 6H).

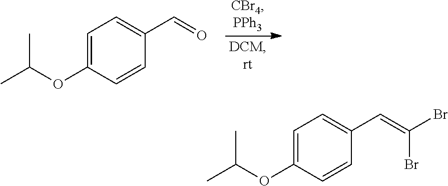

### 1-(2,2-dibromovinyl)-4-isopropoxybenzene

To a solution of carbon tetrabromide (9.09 g, 1.5 Eq, 27.4 mmol) and triphenylphosphine (14.4 g, 3 Eq, 54.8 mmol) in Dichloromethane (183 mL) at room temperature was added 4-isopropoxybenzaldehyde (2.90 mL, 1 Eq, 18.3 mmol) and the mixture was stirred for 16 h at room temperature. Water was added and the mixture was extracted with DCM (3×). The organic phase was washed with brine, dried over Na₂SO₄, filtered and concentrated. Purification by flash column chromatography (SiO₂, 0-10% EtOAc in heptanes) afforded 1-(2,2-dibromovinyl)-4-isopropoxybenzene (4.99 g, 15.6 mmol, 85.3% yield). 1H NMR (400 MHz, CDCl3) δ 7.57-7.43 (m, 2H), 7.39 (s, 1H), 6.91-6.78 (m, 2H), 4.57 (p, J=6.1 Hz, 1H), 1.34 (d, J=6.1 Hz, 6H

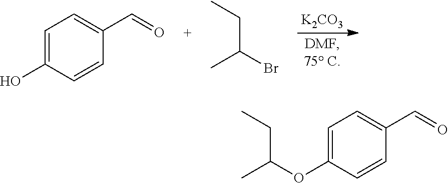

### 4-(sec-butoxy)benzaldehyde

To 4-hydroxybenzaldehyde (12.00 g, 1 Eq, 98.26 mmol) in DMF (50 mL) were added potassium carbonate (27.16 g, 2 Eq, 196.5 mmol) and 2-bromobutane (16.09 mL, 1.5 Eq, 147.4 mmol) and the mixture was heated at 75° C. for 16 h. Diluted with water and DCM and layers separated. The aqueous phase was extracted with DCM (2×) and the combined organics were washed with brine, dried over Na₂SO₄, filtered and concentrated. Purification by flash column chromatography (SiO₂, 0-25% EtOAc in MTBE), afforded 4-(sec-butoxy)benzaldehyde (16.62 g, 93.25 mmol, 94.90% yield). GCMS: Method 8, 2.80 min, 178.1; calcd. 178.2, 1H NMR (400 MHz, CDCl3) δ 9.87 (s, 1H), 7.86-7.75 (m, 2H), 7.02-6.92 (m, 2H), 4.43 (h, J=6.1 Hz, 1H), 1.85-1.63 (m, 2H), 1.34 (d, J=6.1 Hz, 3H), 0.99 (t, J=7.4 Hz, 3H).

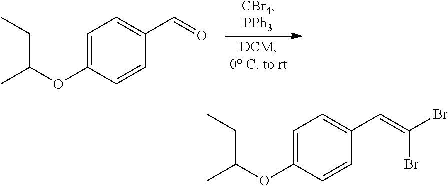

### 1-(sec-butoxy)-4-(2,2-dibromovinyl)benzene

To a solution of carbon tetrabromide (23.7 g, 1.5 Eq, 71.5 mmol) and triphenylphosphine (37.5 g, 3 Eq, 143 mmol) in Dichloromethane (150 mL) at 0° C. was added 4-(sec-butoxy)benzaldehyde (8.50 g, 1 Eq, 47.7 mmol) in Dichloromethane (25 mL) and the mixture was stirred for 1 h at room temperature. Water was added and the mixture was extracted with DCM. The organic phase was washed with brine, dried over Na₂SO₄, filtered and concentrated. Purification by flash column chromatography (SiO₂, 0-15% EtOAc in heptanes) afforded 1-(sec-butoxy)-4-(2,2-dibromovinyl)benzene (13.8 g, 41.3 mmol, 86.6% yield). GCMS: Method 8, 3.44 min, 333.9; calcd. 334.0, 1H NMR (400 MHz, CDCl3) δ 7.53-7.42 (m, 2H), 7.39 (s, 1H), 6.90-6.80 (m, 2H), 4.32 (h, J=6.1 Hz, 1H), 1.81-1.56 (m, 2H), 1.30 (d, J=6.1 Hz, 3H), 0.97 (t, J=7.5 Hz, 3H).

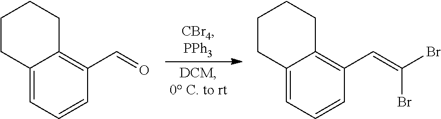

### 5-(2,2-dibromovinyl)-1,2,3,4-tetrahydronaphthalene

To a solution of carbon tetrabromide (7.76 g, 1.5 Eq, 23.4 mmol) and triphenylphosphine (12.3 g, 3 Eq, 46.8 mmol) in Dichloromethane (50 mL) at 0° C. was added 5,6,7,8-tetrahydronaphthalene-1-carbaldehyde (2.50 g, 1 Eq, 15.6 mmol) in Dichloromethane (5 mL) and the mixture was stirred for 16 h at room temperature. Water was added and the mixture was extracted with DCM. The organic phase was washed with brine, dried over Na₂SO₄, filtered and concentrated. Purification by flash column chromatography (SiO₂, 0-15% EtOAc in heptanes) 5-(2,2-dibromovinyl)-1,2,3,4-tetrahydronaphthalene (3.46 g, 10.9 mmol, 70.2% yield). GCMS: Method 8, 3.40 min, 315.9; calcd. 316.0.

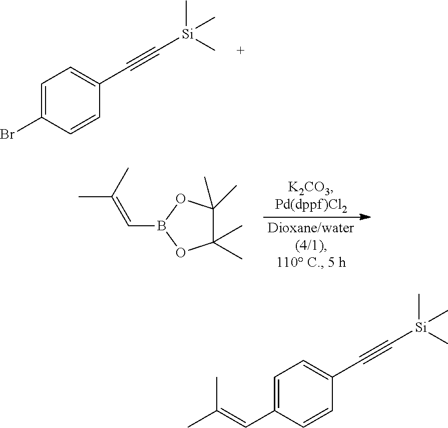 [SMILES: C[Si](C)(C)C#Cc1ccc(Br)cc1 ; CC(C)=CB1OC(C)(C)C(C)(C)O1 ; CC(C)=Cc1ccc(C#C[Si](C)(C)C)cc1]

### Trimethyl((4-(2-methylprop-1-en-1-yl)phenyl)ethynyl)silane

A solution of (2-(4-bromophenyl)ethynyl)trimethylsilane (2.0 g, 1 Eq, 7.9 mmol), 4,4,5,5-tetramethyl-2-(2-methylprop-1-en-1-yl)-1,3,2-dioxaborolane (1.8 mL, 1.1 Eq, 8.7 mmol) and potassium carbonate (3.3 g, 3 Eq, 24 mmol) in 1,4-Dioxane (63 mL)/Water (16 mL) was flushed with Argon for 5 min. Then 1,1′-Bis(diphenylphosphino)ferrocene-palladium(II) dichloride (0.29 g, 0.05 Eq, 0.39 mmol) was added and the mixture was stirred at 110° C. for 5 hours. The mixture was filtered through celite, and the filter cake was rinsed with EtOAc. The organic phase was washed with water and brine, dried over Na₂SO₄, filtered and concentrated. Purification by flash chromatography (SiO₂, 0-100% MTBE in heptanes), afforded trimethyl((4-(2-methylprop-1-en-1-yl)phenyl)ethynyl)silane (1.8 g, 6.5 mmol, 82% yield, 82% Purity). GCMS: Method 8, 4.58 min, 228.2; calcd. 228.4. 1H NMR (400 MHz, CDCl3) δ 7.46-7.35 (m, 2H), 7.18-7.11 (m, 2H), 6.23 (d, J=2.4 Hz, 1H), 1.88 (dd, J=19.0, 1.4 Hz, 6H), 0.24 (s, 9H).

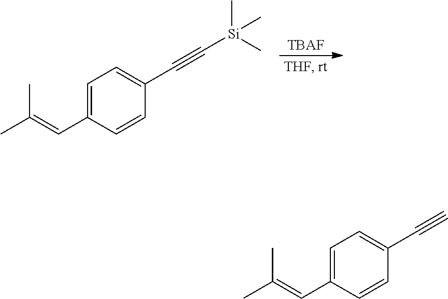

### 1-ethynyl-4-(2-methylprop-1-en-1-yl)benzene

To a solution of trimethyl((4-(2-methylprop-1-en-1-yl)phenyl)ethynyl)silane (1.8 g, 82% Wt., 1 Eq, 6.5 mmol) in Tetrahydrofuran (65 mL) was added TBAF, 1M in THF (7.8 mL, 1.2 Eq, 7.8 mmol) and the mixture was stirred at room temperature for 1 hour. The mixture was concentrated and purified further by flash chromatography (SiO₂, 0-100% MTBE in heptanes, then 0-20% MTBE in heptanes), affording 1-ethynyl-4-(2-methylprop-1-en-1-yl)benzene (0.63 g, 3.8 mmol, 58% yield, 93% Purity). 1H NMR (400 MHz, CDCl3) δ 7.47-7.37 (m, 2H), 7.21-7.14 (m, 2H), 6.24 (d, J=1.9 Hz, 1H), 3.06 (s, 1H), 1.89 (dd, J=18.6, 1.4 Hz, 6H).

### Example 54: Odor Profile Determination

Odor profile for selected compounds within the scope of the disclosure is determined using assessments by one Master Perfumer and 4-6 trained sensory panelists. Sensory panelists are trained on our internal taxonomy with reference materials that include 11 grand families, and 62 subfamilies. Panelists are experienced in, but not trained in, an additional several hundred odor descriptors. Panelists are trained on usage of a rating scale, which also includes internal references. Panelists are not advanced to compound evaluation until they have completed and received a passing score on a Final Exam on the taxonomy that was curated by our perfumery team.

A single score is generated for the sensory panel by taking the arithmetic mean of all panelist scores for a given sample, for a given attribute. Alternatively, a single score may be generated by fitting all panelist scores to a model that can correct for differences in the intercept or slope of the latent function through which each panelist produces a rating given a percept, i.e. correction for inter-panelist variation.

The Master Perfumer, possessing a deep knowledge of fragrance ingredients, high level technical expertise, and who is recognized for their experience in creating complex perfumes, reviews, may augment sensory panel scores for improved accuracy.

Test samples are dissolved at a concentration of 10% w/v in ethanol. At time 0 (t=0), new test blotters (White Paper Paddle Shaped Perfumery Blotters, measuring 5×0.5 inches) are dipped into the 10% solution of the text compound. Odor descriptions at time t=0 are captured within 1-2 minutes of wetting the blotter to allow for evaporation of most of the ethanol solvent, permitting a more accurate determination of the test compound's odor. Assessments are made at ambient temperature in a benchtop laboratory setting. For the longevity determination, blotters are stored in open air on the laboratory benchtop between assessments (no jar or other enclosure is used).

The results are shown in the table below:

Intensity: 1-10 (max) at t = 0, 10% EtOH solution on blotter paper
Longevity: 10% EtOH solution on blotter paper
+ = detectable 0-1 h; ++ = detectable 1 h-6 h; +++ = detectable 6 h-24 h

| Ex. | | Primary | Intensity | Longevity |
|---|---|---|---|---|
| No. | Structure | Notes | (1-10) | (+/++/+++) |
| 1 | 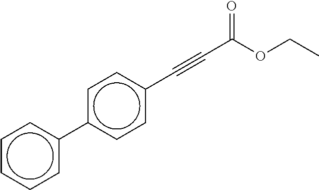 | mineral marine metallic fruity | 4 | + |
| 2 | 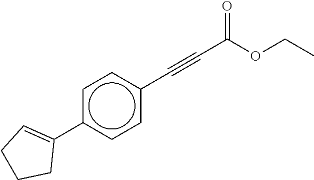 | floral jasmin fruity | 4 | ++ |
| 3 | 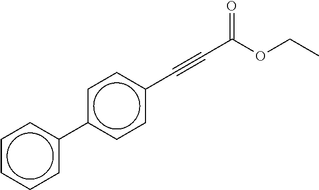 | floral jasmin woody creamy | 4 | + |
| 4 | 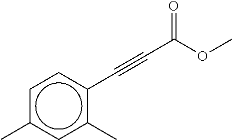 | floral hawthorn citrus orange- flower fresh | 5 | ++ |
| 5 | 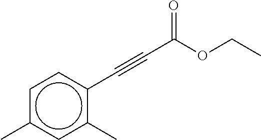 | floral salicylate ink hawthorn honey green mimosa | 5 | + |
| 6 | 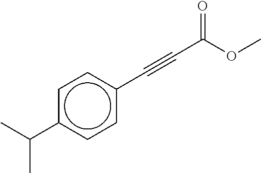 | floral spicy cumin violet | 6 | + |
| 7 | 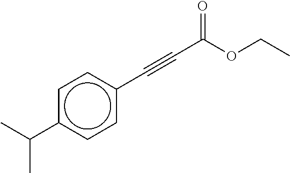 | floral violet orris woody | 6 | + |
| 8 | 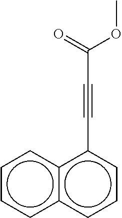 | floral | 4 | ++ |
| 9 | 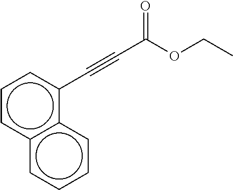 | floral | 4 | ++ |
| 10 | 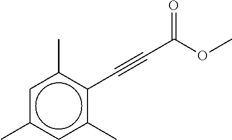 | woody floral sweet-pea white- flour | 6 | +++ |
| 11 | 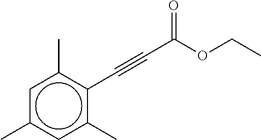 | fruity appley musty | 6 | +++ |
| 12 | 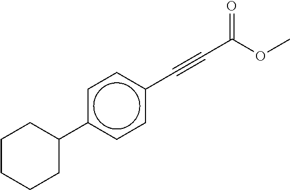 | rosy mushroom citrus | 5 | + |
| 13 | 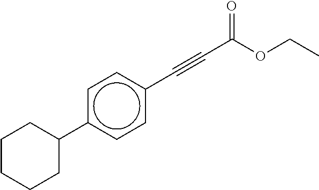 | weak | 4 | + |
| 14 | 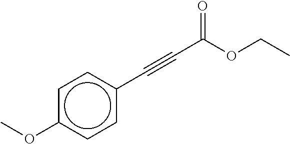 | sweet fruity | 6 | ++ |
| 15 | 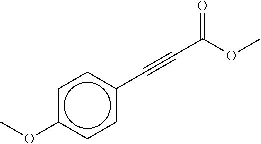 | sweet | 6 | ++ |
| 16 | 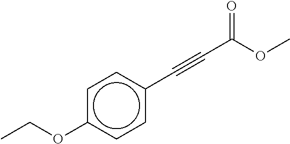 | woody smokey salty | 6 | ++ |
| 17 | 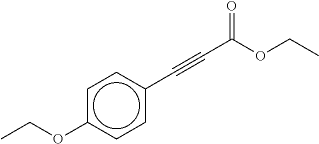 | fruity | 5 | ++ |
| 18 | 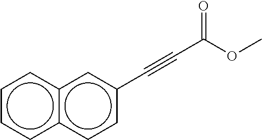 | faint | 4 | + |
| 19 | 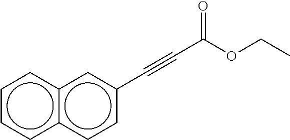 | faint | 4 | + |
| 20 | 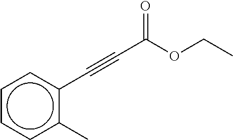 | floral jasmine woody masculine lavender salicylate | 7 | +++ |
| 21 | 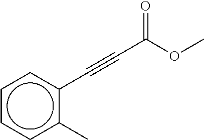 | floral moss woody rosy | 7 | +++ |
| 22 | 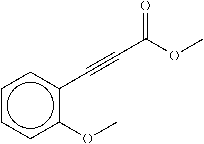 | straw | 6 | ++ |
| 23 | 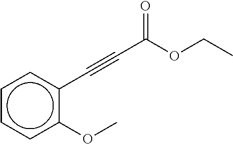 | floral hay sweet nutty starchy | 6 | ++ |
| 24 | 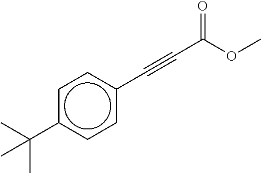 | fruity herbal | 6 | +++ |
| 25 | 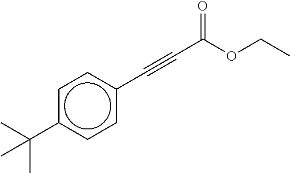 | apple ripe strawberry tutti fruity | 5 | +++ |
| 26 | 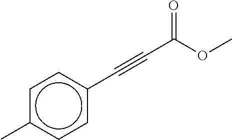 | fruity greasy soulful peach | 8 | +++ |
| 27 | 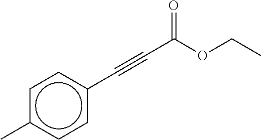 | fruity green | 8 | +++ |
| 28 | 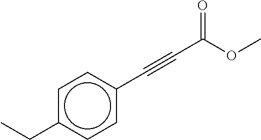 | woody floral | 7 | +++ |
| 29 | 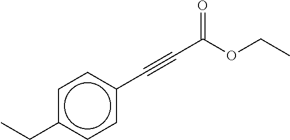 | vanilla fruity sweet | 7 | +++ |
| 30 | 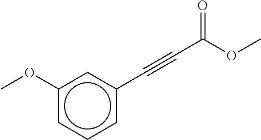 | sweet vanilla fresh mushroom | 5 | ++ |
| 31 | 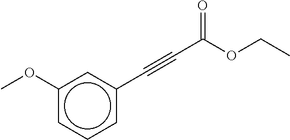 | watery sweet | 5 | ++ |
| 32 | 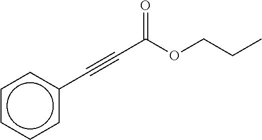 | floral jasmin watery peppery | 6 | +++ |
| 33 | 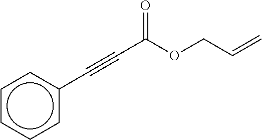 | jasmin strawberry | 8 | +++ |
| 34 | 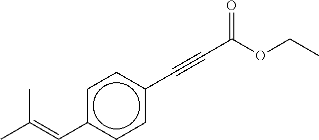 | floral fruity animalic | 5 | ++ |
| 35 | 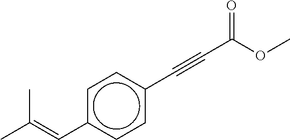 | animalic floral fruity | 5 | ++ |
| 36 | 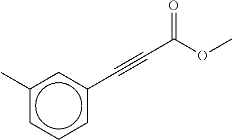 | floral muguet watery green pear | 7 | +++ |
| 37 | 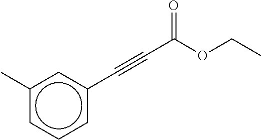 | orange apple | 7 | +++ |
| 38 | 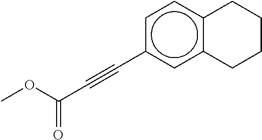 | fruity weak | 4 | + |
| 39 | 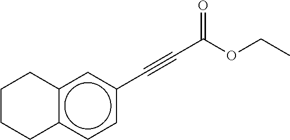 | fruity pineapple | 4 | + |
| 40 | 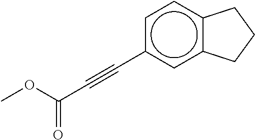 | floral green | 5 | + |
| 41 | 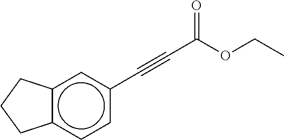 | industrial | 5 | + |
| 42 | 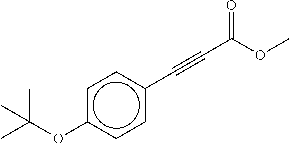 | fatty industrial nutty | 4 | ++ |
| 43 | 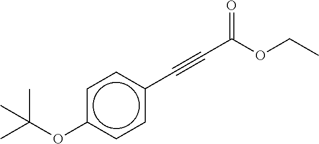 | white- flower sweet | 4 | ++ |
| 44 | 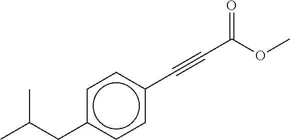 | marine seaweed green muguet | 6 | +++ |
| 45 | 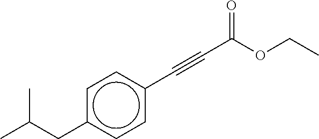 | fruity appley pineapple | 6 | +++ |
| 46 | 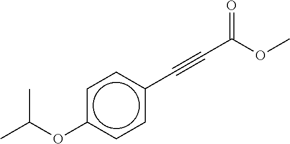 | fatty oily | 5 | ++ |
| 47 | 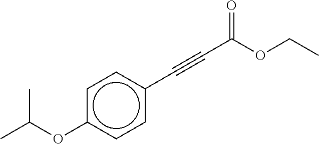 | floral rosy green narcissus | 5 | ++ |
| 48 | 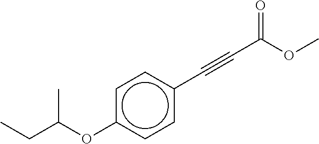 | sweet lactonic | 6 | ++ |
| 49 | 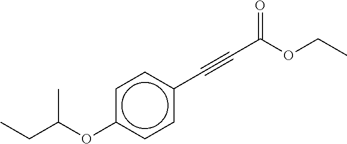 | floral | 3 | + |
| 50 | 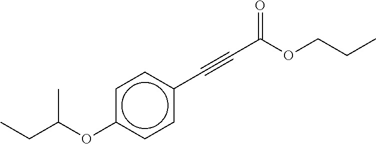 | floral | 3 | + |
| 51 | 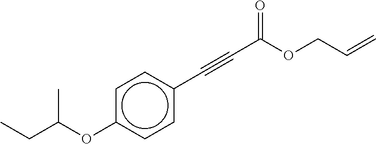 | wet-paper citrus | 2 | + |
| 52 | 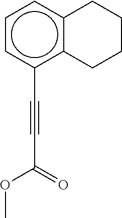 | weak | 2 | + |
| 53 | 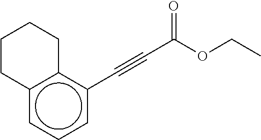 | weak | 2 | + |
| 54 | 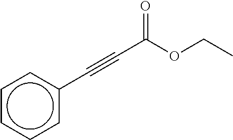 | jasmine fruity | 8 | +++ |
| 55 | 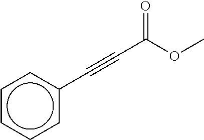 | floral white soulful vanilla green | 8 | +++ |

The Examples provided herein are exemplary only and are not intended to be limiting in any way to the various aspects and embodiments of the invention described herein.

1. A compound of Formula I: 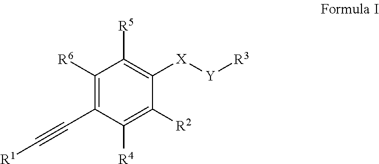
wherein:
R¹ is CHO, COOH, or COOR^{a};
R² is H, C₁₋₃ alkyl (e.g., CH₃), OH, CHO, or OC₁₋₃ alkyl (e.g., OCH₃);
X is selected from —O—, —CH₂—, —C(O)—, CH(OH), and —CH(OR)—;
Y is selected from —CH₂CH₂CH₂CH₂—, —CH₂CH₂OCH₂—, —CH₂OCH₂CH₂—, —OCH₂CH₂CH₂—, —CH₂CH₂CH₂—, —CH₂CH₂O—, —CH₂OCH₂—, —OCH₂CH₂—, —CH₂CH₂CH₂CH₂CH₂—, —OCH₂CH₂CH₂CH₂—, —CH₂OCH₂CH₂CH₂—, —CH₂CH₂OCH₂CH₂—, —CH₂CH₂CH₂OCH₂—, —CH₂CH₂CH₂CH₂O—, —CH₂CH₂CH₂CH₂CH₂CH₂—, —OCH₂CH₂CH₂CH₂CH₂—, —CH₂OCH₂CH₂CH₂CH₂—, —CH₂CH₂OCH₂CH₂CH₂—, —CH₂CH₂CH₂OCH₂CH₂—, —CH₂CH₂CH₂CH₂OCH₂—, —CH₂CH₂CH₂CH₂CH₂O—, —CH₂CH₂CH₂CH₂CH₂CH₂CH₂—, —OCH₂CH₂CH₂CH₂CH₂CH₂—, —CH₂OCH₂CH₂CH₂CH₂CH₂—, —CH₂CH₂OCH₂CH₂CH₂CH₂—, —CH₂CH₂CH₂OCH₂CH₂CH₂—, —CH₂CH₂CH₂CH₂OCH₂CH₂—, —CH₂CH₂CH₂CH₂CH₂OCH₂—, and —CH₂CH₂CH₂CH₂CH₂CH₂O—,
wherein said group Y is optionally substituted by one or more groups selected from C₁₋₃ alkyl (e.g., CH₃), OH, CHO, and OC₁₋₃ alkyl (e.g., OCH₃);
or X—Y is absent;
R³ is selected from H, R^{b}, OH, OR^{b}, OC(O)R^{b}, CN, COOH, COOR^{b}, C(O)R^{b}, and CHO;
R⁴, R⁵, and R⁶ are each independently selected from H, C₁₋₃ alkyl (e.g., CH₃), OH, CHO, or OC₁₋₃ alkyl (e.g., OCH₃); and
each R^{a} and R^{b} is independently optionally substituted C₁₋₆alkyl (e.g., CH₃ or CH₂CH₃), C₂₋₆alkenyl (e.g., allyl or 2-methylprop-1-enyl), CH₂C₃₋₆cycloalkyl (e.g., cyclopropylmethyl), C₃₋₆cycloalkyl (e.g., cyclopropyl), C₄₋₆cycloalkenyl (e.g., cyclopentenyl), optionally substituted phenyl, or optionally substituted benzyl;
provided that the compound is not ethyl 3-phenylpropiolate, methyl 3-phenylpropiolate, or 3-phenylpropiolic acid, and wherein the compound is not a compound wherein:
(a) R¹ and R² are H, and X—Y—R³ is n-butyl, n-pentyl, n-hexyl, or n-nonyl;
(b) R¹ and R² are H, X is —C(O)—, and Y—R³ is n-butyl, n-octyl, or n-undecyl;
(c) R¹ and R² are H, X is —CH(OH)—, and Y—R³ is n-butyl, n-octyl, or n-undecyl;
(d) R¹ and R² are H, X is —O—, and Y—R³ is n-butyl, n-pentyl, or n-hexyl;
or a compound of Formula II: 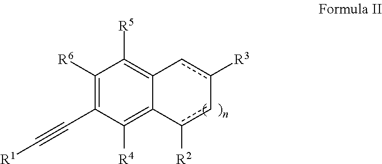
wherein:
n is 0 or 1;
R¹ is CHO, COOH, or COOR^{a};
R² is H, C₁₋₃ alkyl (e.g., CH₃), OH, CHO, or OC₁₋₃ alkyl (e.g., OCH₃);
R³ is selected from H, R^{b}, OH, OR^{b}, OC(O)R^{b}, CN, COOH, COOR^{b}, C(O)R^{b}, and CHO;
R⁴, R⁵, and R⁶ are each independently selected from H, C₁₋₃ alkyl (e.g., CH₃), OH, CHO, or OC₁₋₃ alkyl (e.g., OCH₃); and
each R^{a} and R^{b} is independently optionally substituted C₁₋₆alkyl (e.g., CH₃ or CH₂CH₃), C₂₋₆alkenyl (e.g., allyl or 2-methylprop-1-enyl), CH₂C₃₋₆cycloalkyl (e.g., cyclopropylmethyl), C₃₋₆cycloalkyl (e.g., cyclopropyl), C₄₋₆cycloalkenyl (e.g., cyclopentenyl), optionally substituted phenyl, or optionally substituted benzyl;
or a compound of Formula III: 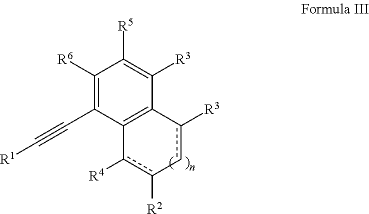
wherein:
n is 0 or 1;
R¹ is CHO, COOH, or COOR^{a};
R² is H, C₁₋₃ alkyl (e.g., CH₃), OH, CHO, or OC₁₋₃ alkyl (e.g., OCH₃);
R³ is selected from H, R^{b}, OH, OR^{b}, OC(O)R^{b}, CN, COOH, COOR^{b}, C(O)R^{b}, and CHO;
R⁴, R⁵, and R⁶ are each independently selected from H, C₁₋₃ alkyl (e.g., CH₃), OH, CHO, or OC₁₋₃ alkyl (e.g., OCH₃); and
each R^{a} and R^{b} is independently optionally substituted C₁₋₆alkyl (e.g., CH₃ or CH₂CH₃), C₂₋₆alkenyl (e.g., allyl or 2-methylprop-1-enyl), CH₂C₃₋₆cycloalkyl (e.g., cyclopropylmethyl), C₃₋₆cycloalkyl (e.g., cyclopropyl), C₄₋₆cycloalkenyl (e.g., cyclopentenyl), optionally substituted phenyl, or optionally substituted benzyl.

2. (canceled)

3. A flavor composition and/or fragrance composition comprising a compound of Formula I: 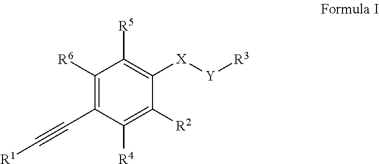
wherein:
R¹ is CHO, COOH, or COOR^{a};
R² is H, C₁₋₃ alkyl (e.g., CH₃), OH, CHO, or OC₁₋₃ alkyl (e.g., OCH₃);
X is selected from —O—, —CH₂—, —C(O)—, —CH(OH)—, and —CH(OR)—;
Y is selected from —CH₂CH₂CH₂CH₂—, —CH₂CH₂OCH₂—, —CH₂OCH₂CH₂—, —OCH₂CH₂CH₂—, —CH₂CH₂CH₂—, —CH₂CH₂O—, —CH₂OCH₂—, —OCH₂CH₂—, —CH₂CH₂CH₂CH₂CH₂—, —OCH₂CH₂CH₂CH₂—, —CH₂OCH₂CH₂CH₂—, —CH₂CH₂OCH₂CH₂—, —CH₂CH₂CH₂OCH₂—, —CH₂CH₂CH₂CH₂O—, —CH₂CH₂CH₂CH₂CH₂CH₂—, —OCH₂CH₂CH₂CH₂CH₂—, —CH₂OCH₂CH₂CH₂CH₂—, —CH₂CH₂OCH₂CH₂CH₂—, —CH₂CH₂CH₂OCH₂CH₂—, —CH₂CH₂CH₂CH₂OCH₂—, —CH₂CH₂CH₂CH₂CH₂O—, —CH₂CH₂CH₂CH₂CH₂CH₂CH₂—, —OCH₂CH₂CH₂CH₂CH₂CH₂—, —CH₂OCH₂CH₂CH₂CH₂CH₂—, —CH₂CH₂OCH₂CH₂CH₂CH₂—, —CH₂CH₂CH₂OCH₂CH₂CH₂—, —CH₂CH₂CH₂CH₂OCH₂CH₂—, —CH₂CH₂CH₂CH₂CH₂OCH₂—, and —CH₂CH₂CH₂CH₂CH₂CH₂O—,
wherein said group Y is optionally substituted by one or more groups selected from C₁₋₃ alkyl (e.g., CH₃), OH, CHO, and OC₁₋₃ alkyl (e.g., OCH₃);
or X—Y is absent;
R³ is selected from H, R^{b}, OH, OR^{b}, OC(O)R^{b}, CN, COOH, COOR^{b}, C(O)R^{b}, and CHO;
R⁴, R⁵, and R⁶ are each independently selected from H, C₁₋₃ alkyl (e.g., CH₃), OH, CHO, or OC₁₋₃ alkyl (e.g., OCH₃); and
each R^{a} and R^{b} is independently optionally substituted C₁₋₆alkyl (e.g., CH₃ or CH₂CH₃), C₂₋₆alkenyl (e.g., allyl or 2-methylprop-1-enyl), CH₂C₃₋₆cycloalkyl (e.g., cyclopropylmethyl), C₃₋₆cycloalkyl (e.g., cyclopropyl), C₄₋₆cycloalkenyl (e.g., cyclopentenyl), optionally substituted phenyl, and optionally substituted benzyl;
or a compound of Formula II: 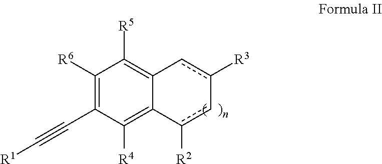
wherein:
n is 0 or 1;
R¹ is CHO, COOH, or COOR^{a};
R² is H, C₁₋₃ alkyl (e.g., CH₃), OH, CHO, or OC₁₋₃ alkyl (e.g., OCH₃);
R³ is selected from H, R^{b}, OH, OR^{b}, OC(O)R^{b}, CN, COOH, COOR^{b}, C(O)R^{b}, and CHO;
R⁴, R⁵, and R⁶ are each independently selected from H, C₁₋₃ alkyl (e.g., CH₃), OH, CHO, or OC₁₋₃ alkyl (e.g., OCH₃); and
each R^{a} and R^{b} is independently optionally substituted C₁₋₆alkyl (e.g., CH₃ or CH₂CH₃), C₂₋₆alkenyl (e.g., allyl or 2-methylprop-1-enyl), CH₂C₃₋₆cycloalkyl (e.g., cyclopropylmethyl), C₃₋₆cycloalkyl (e.g., cyclopropyl), C₄₋₆cycloalkenyl (e.g., cyclopentenyl), optionally substituted phenyl, or optionally substituted benzyl;
or a compound of Formula III: 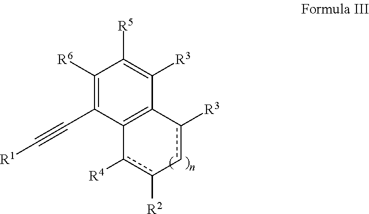
wherein:
n is 0 or 1;
R¹ is CHO, COOH, or COOR^{a};
R² is H, C₁₋₃ alkyl (e.g., CH₃), OH, CHO, or OC₁₋₃ alkyl (e.g., OCH₃);
R³ is selected from H, R^{b}, OH, OR^{b}, OC(O)R^{b}, CN, COOH, COOR^{b}, C(O)R^{b}, and CHO;
R⁴, R⁵, and R⁶ are each independently selected from H, C₁₋₃ alkyl (e.g., CH₃), OH, CHO, or OC₁₋₃ alkyl (e.g., OCH₃); and
each R^{a} and R^{b} is independently optionally substituted C₁₋₆alkyl (e.g., CH₃ or CH₂CH₃), C₂₋₆alkenyl (e.g., allyl or 2-methylprop-1-enyl), CH₂C₃₋₆cycloalkyl (e.g., cyclopropylmethyl), C₃₋₆cycloalkyl (e.g., cyclopropyl), C₄₋₆cycloalkenyl (e.g., cyclopentenyl), optionally substituted phenyl, or optionally substituted benzyl in admixture with one or more non-toxic, orally acceptable, pharmaceutically acceptable, cosmetically acceptable, or acceptable for a household product, carriers or excipients.

4. The composition of claim 3, wherein the compound of Formula I is a compound of Formula Ia: 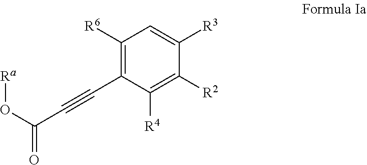
wherein R^{a} is selected from methyl, ethyl, propyl, isopropyl, n-butyl, sec-butyl, isobutyl, and tert-butyl, R², R⁴ and R⁶ are each independently H, C₁₋₃ alkyl (e.g., CH₃) or OC₁₋₃ alkyl (e.g., OCH₃), and R³ is R^{b} or OR^{b} and R^{b} is selected from methyl, ethyl, propyl, isopropyl, n-butyl, sec-butyl, isobutyl, tert-butyl, allyl, 2-methylprop-1-enyl, phenyl, cyclopentyl, or cyclohexenyl.

5. The composition of claim 3, wherein the compound of Formula II is a compound of Formula IId, IIe, or IIf: 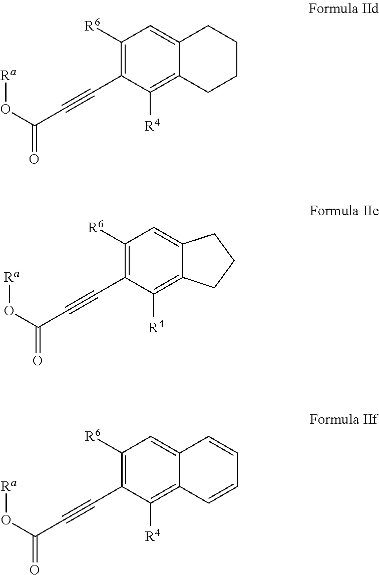
wherein R^{a} is selected from methyl, ethyl, propyl, isopropyl, n-butyl, sec-butyl, isobutyl, and tert-butyl, and R⁴ and R⁶ are each independently H, C₁₋₃ alkyl (e.g., CH₃) or OC₁₋₃ alkyl (e.g., OCH₃).

6. The composition of claim 3, wherein the compound of Formula III is a compound of Formula IIId, IIIe, or IIIf: 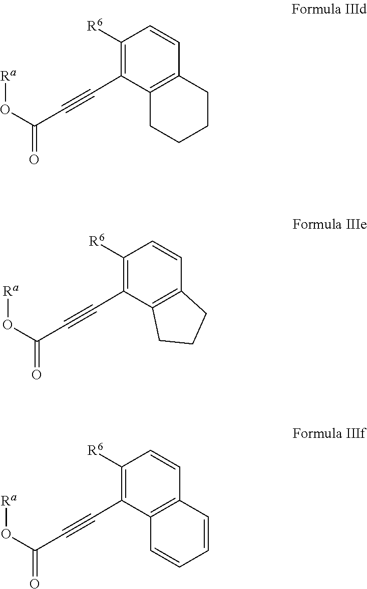
wherein R^{a} is selected from methyl, ethyl, propyl, isopropyl, n-butyl, sec-butyl, isobutyl, and tert-butyl, and R⁶ is H, C₁₋₃ alkyl (e.g., CH₃) or OC₁₋₃ alkyl (e.g., OCH₃).

7. The composition of claim 3, wherein the compound is selected from the group consisting of:

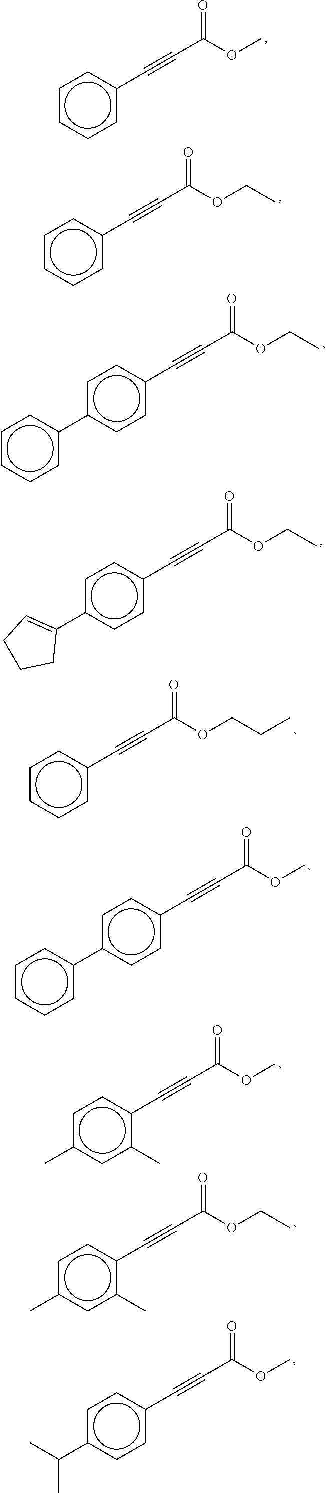

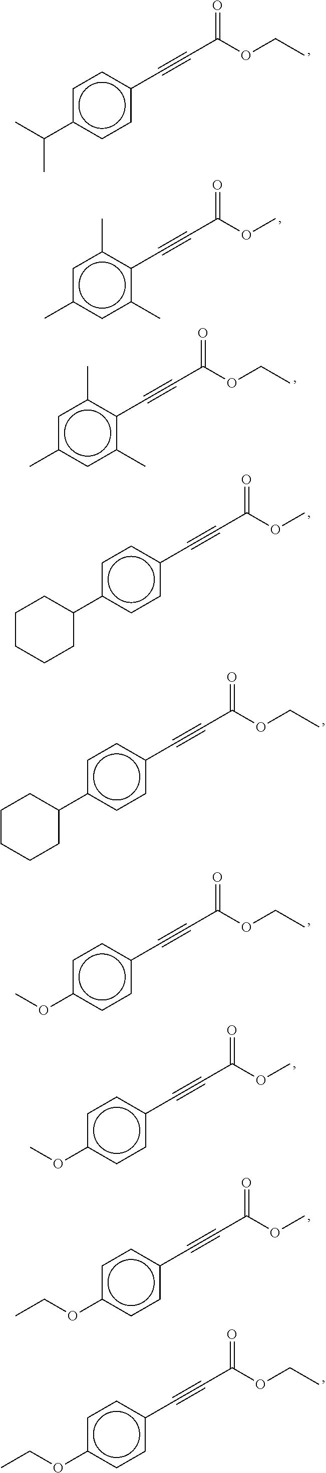

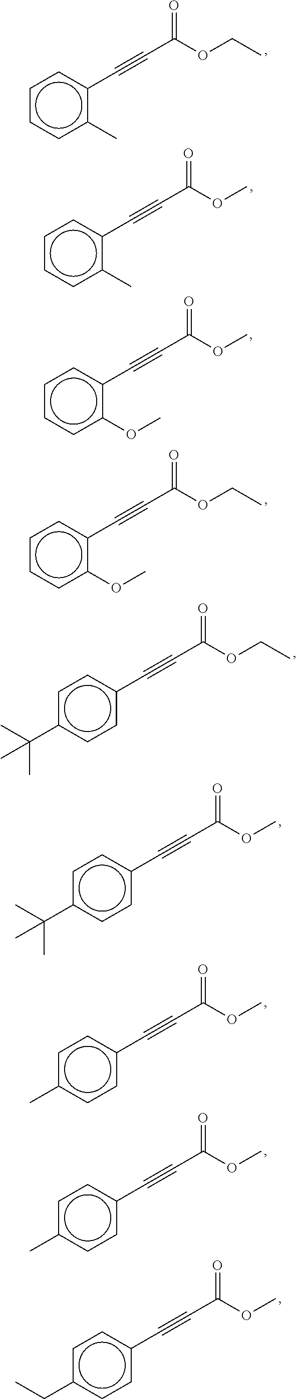

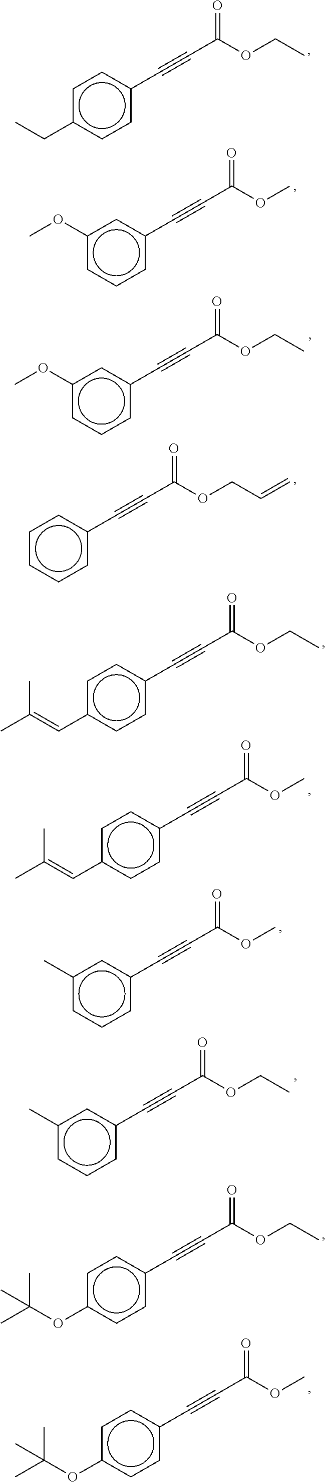

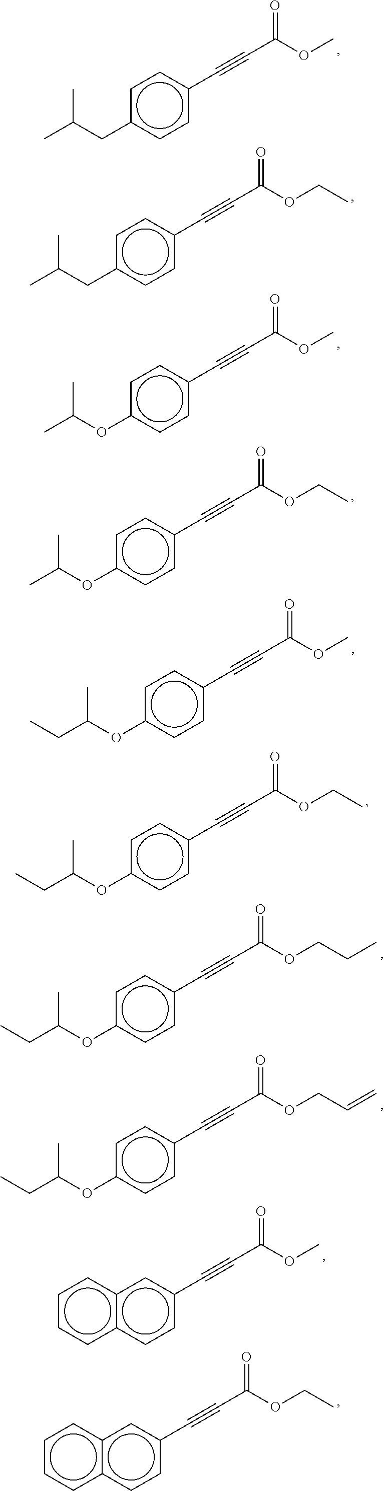

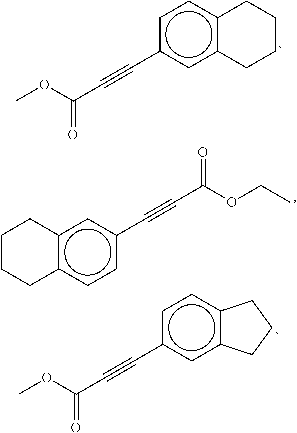

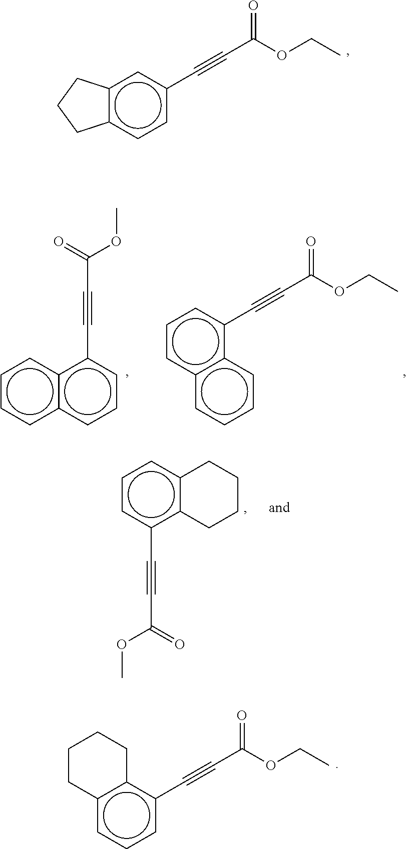

8. The composition of claim 3, wherein the compound is selected from the group consisting of:

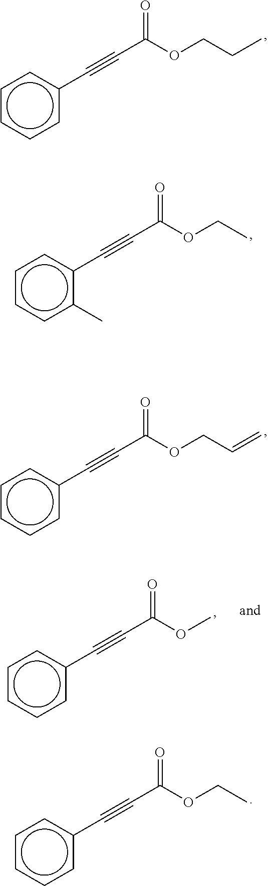

9. The composition of claim 3, wherein the composition further comprises one or more solvents.

10. The composition of claim 3, wherein the composition further comprises one or more other flavors or fragrances.

11. A product comprising the composition of claim 3 wherein the product is selected from the following: personal care products (e.g., a soap, skin cream or lotion, balm, shampoo, body wash, shower gel, hydrating cream, deodorant, antiperspirant, after-shave lotion, cologne, perfume, or other hair care or skin care product), sunscreens, insect repellants and insecticides, detergents, household cleaning agents (e.g., a surface cleaner, a metal cleaner, a wood cleaner, a glass cleaner, a body cleaner such as a soap, a dish-washing detergent, or a laundry detergent), air fresheners, room sprays, pomanders, candles, cosmetics (e.g., perfumes, colognes, nail polish, eye liner, mascara, lipstick, foundation, concealer, blush, bronzer, eye shadow, lip liner, lip balm), toilet waters, talcum powders, and pet litter.

12. The composition of claim 3, wherein the compound of Formula I is a compound of Formula Ia: 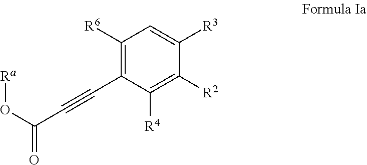
wherein:
R² is H, C₁₋₃ alkyl (e.g., CH₃), or OC₁₋₃ alkyl (e.g., OCH₃);
R³ is selected from H, R^{b}, and OR^{b};
R⁴ and R⁶ are each independently selected from H, C₁₋₃ alkyl (e.g., CH₃), or OC₁₋₃ alkyl (e.g., OCH₃);
R^{a} is C₁₋₆alkyl (e.g., CH₃ or CH₂CH₃) or C₂₋₆alkenyl (e.g., allyl or 2-methylprop-1-enyl); and
R^{b} is C₁₋₆alkyl (e.g., CH₃ or CH₂CH₃), C₂₋₆alkenyl (e.g., allyl or 2-methylprop-1-enyl), CH₂C₃₋₆cycloalkyl (e.g., cyclopropylmethyl), C₃₋₆cycloalkyl (e.g., cyclopropyl), or C₄₋₆cycloalkenyl (e.g., cyclopentenyl).

13. The composition of claim 12, wherein R², R⁴ and R⁶ are each independently H, C₁₋₃ alkyl (e.g., CH₃) or OC₁₋₃ alkyl (e.g., OCH₃), R³ is R^{b} or OR^{b} and R^{b} is selected from methyl, ethyl, propyl, isopropyl, n-butyl, sec-butyl, isobutyl, tert-butyl, allyl, or 2-methylprop-1-enyl; R^{a} is selected from methyl, ethyl, propyl, isopropyl, n-butyl, sec-butyl, isobutyl, and tert-butyl.

14. The composition of claim 3, wherein the compound is selected from the group consisting of:

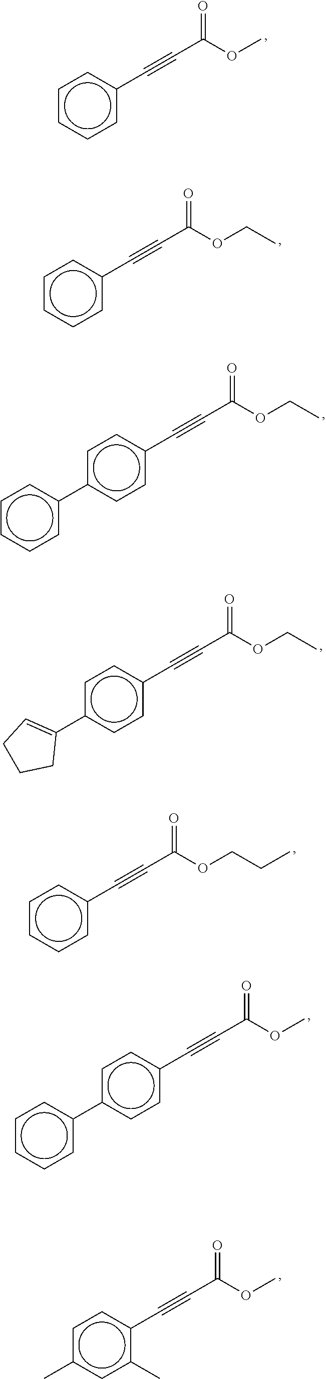

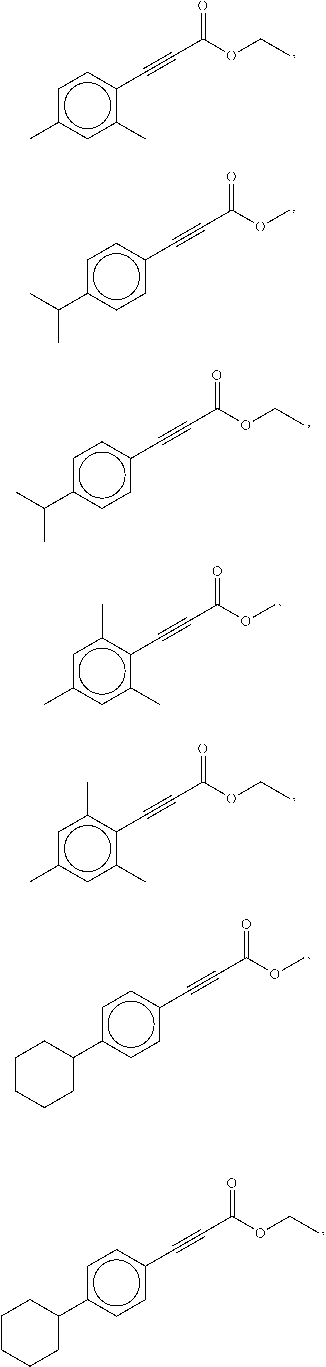

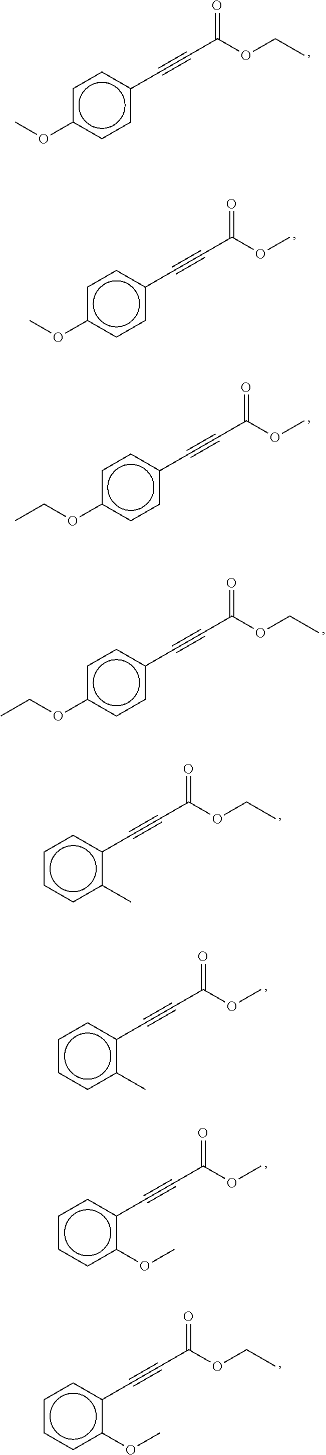

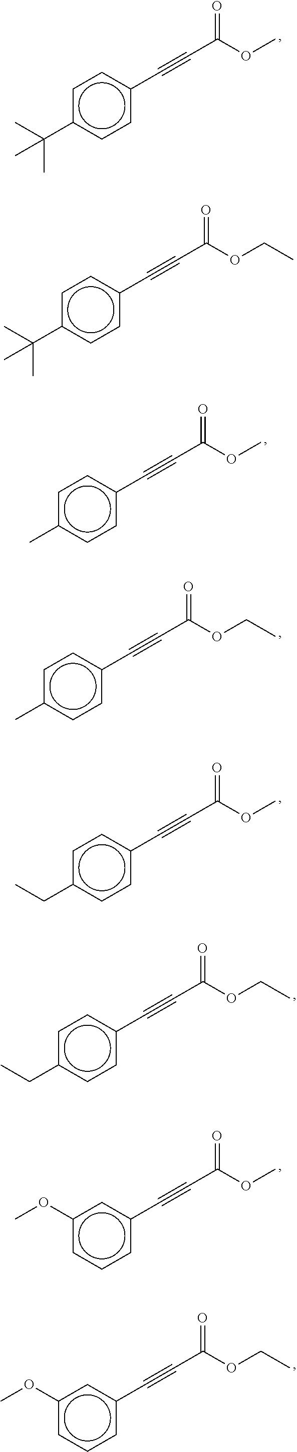

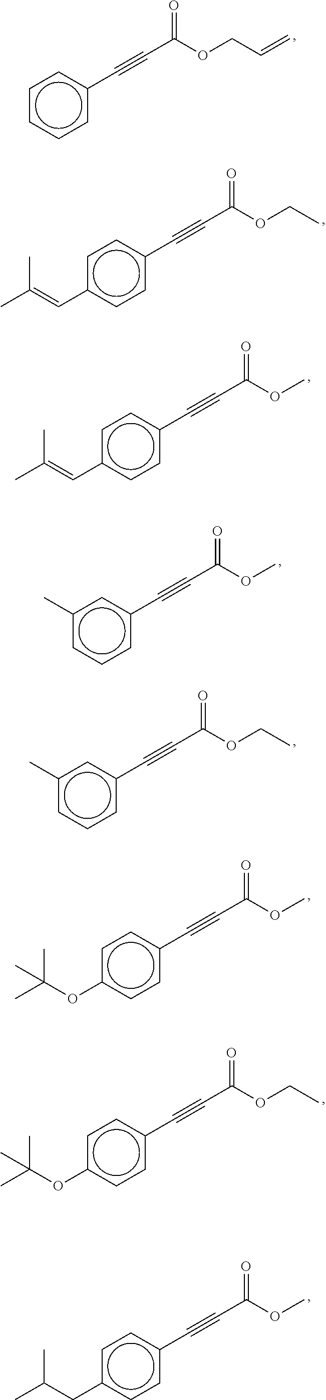

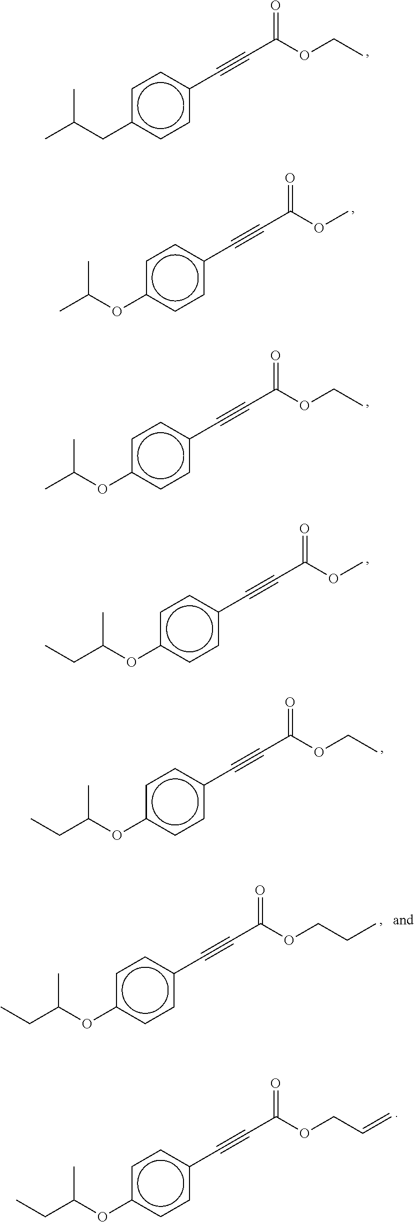

15. The composition of claim 3, wherein the compound is selected from the group consisting of:

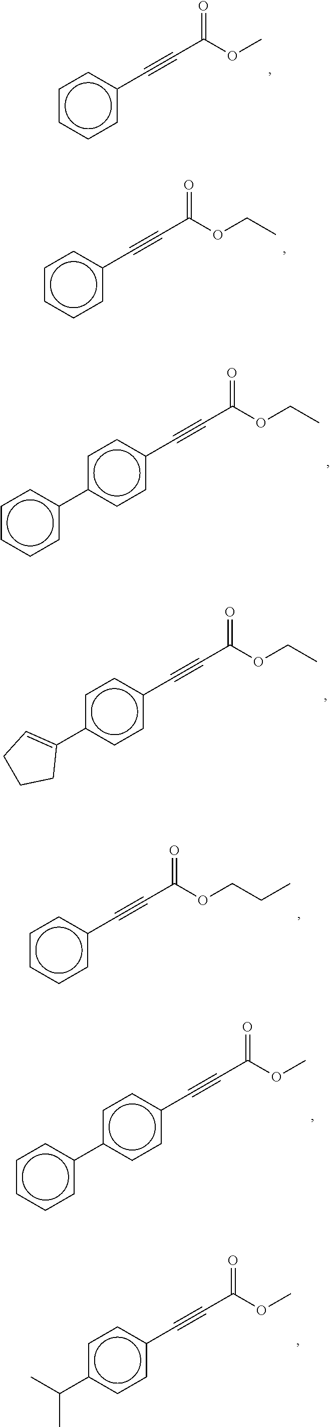

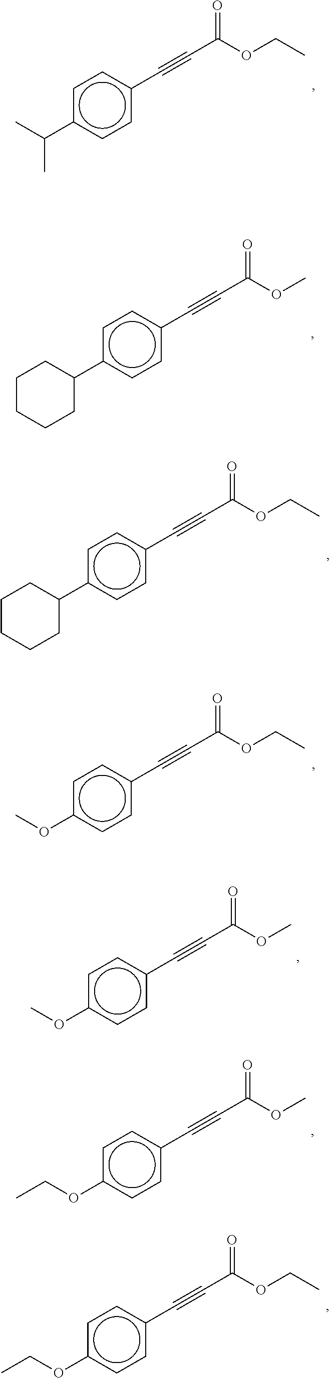

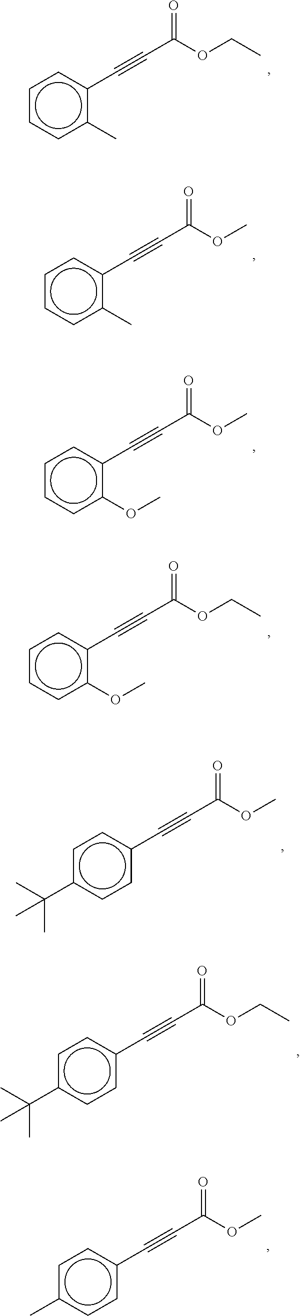

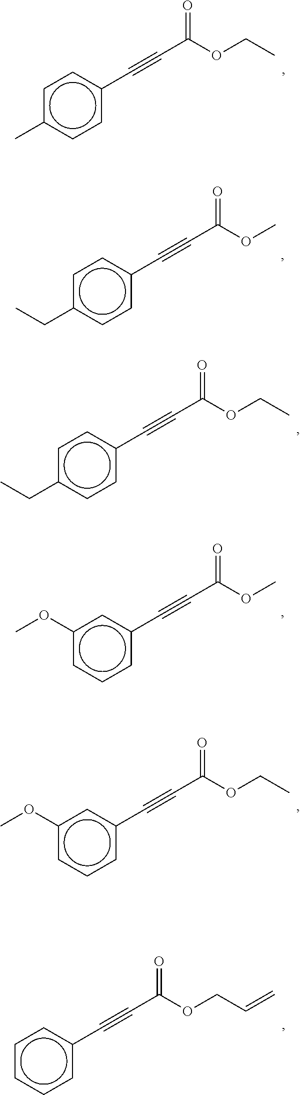

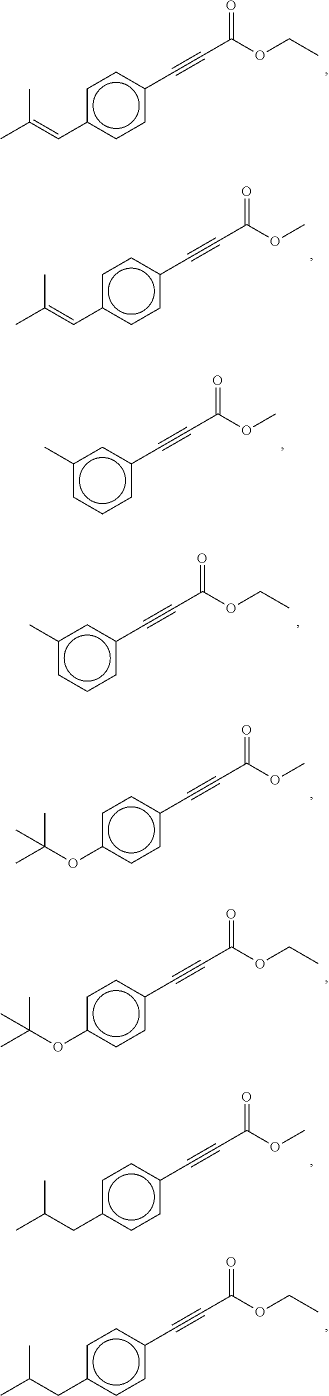

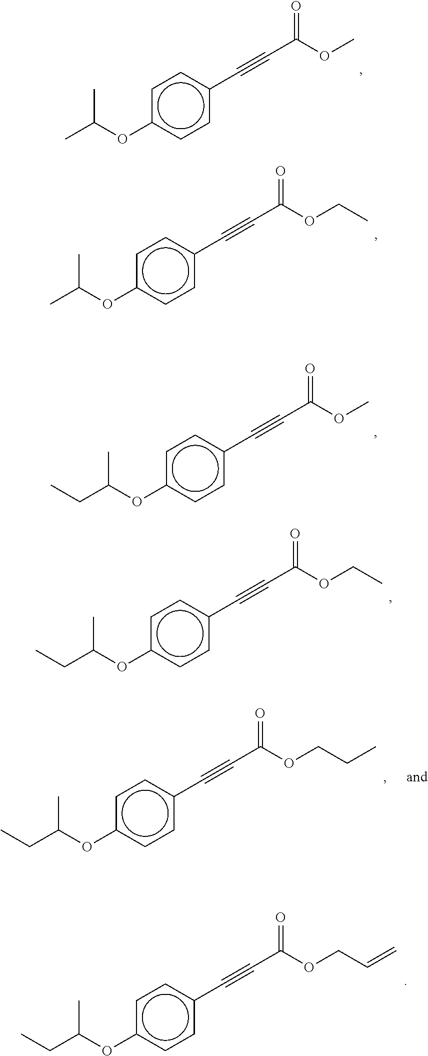

16. The composition of claim 3, wherein the compound is selected from the group consisting of:

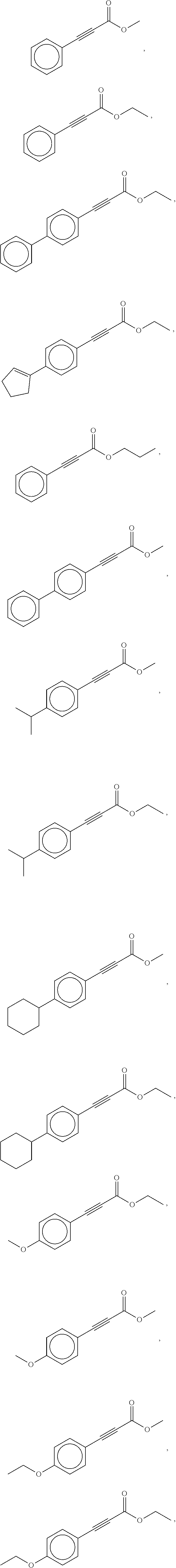

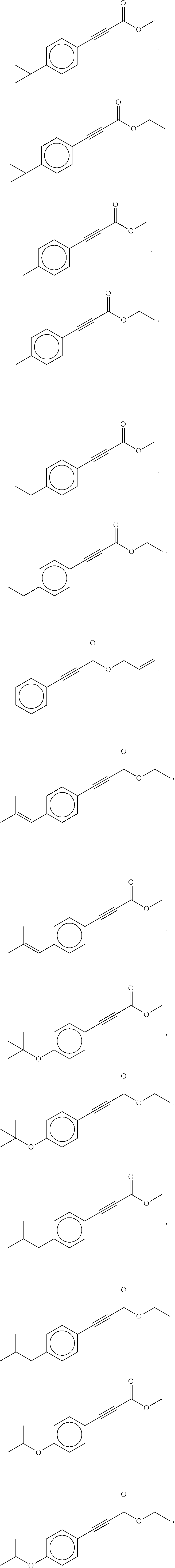

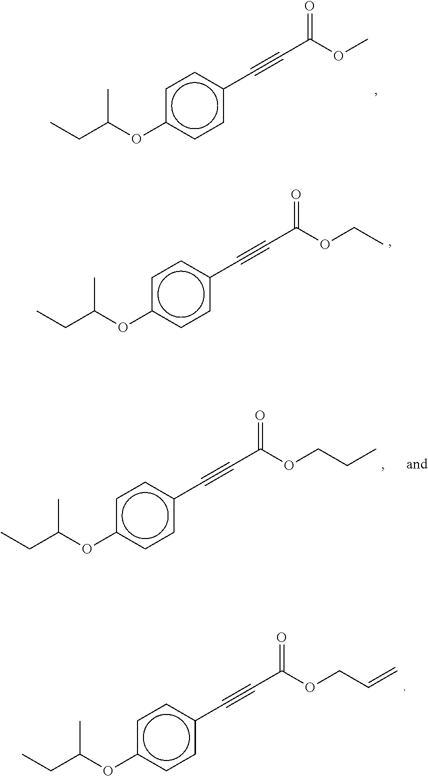

17. The composition of claim 3, wherein the compound is selected from the group consisting of:

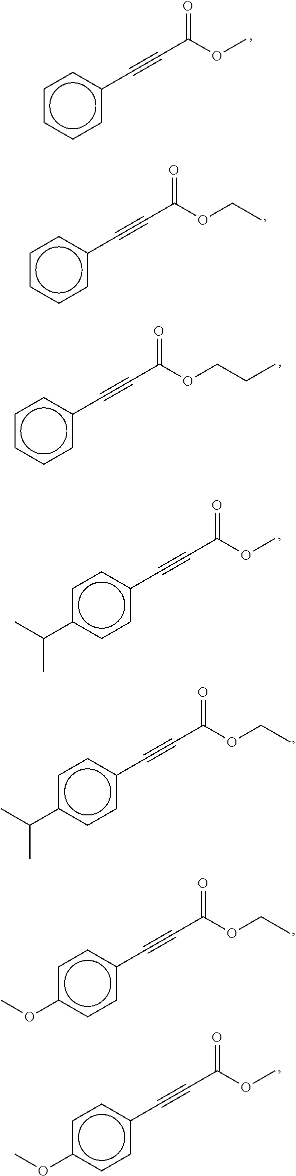

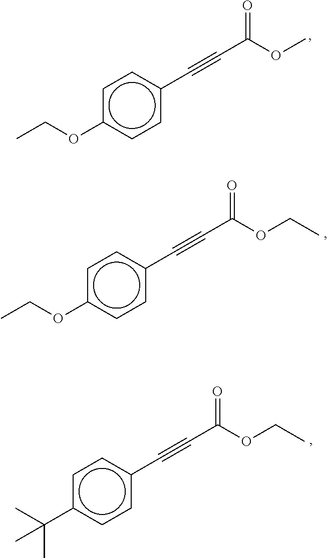

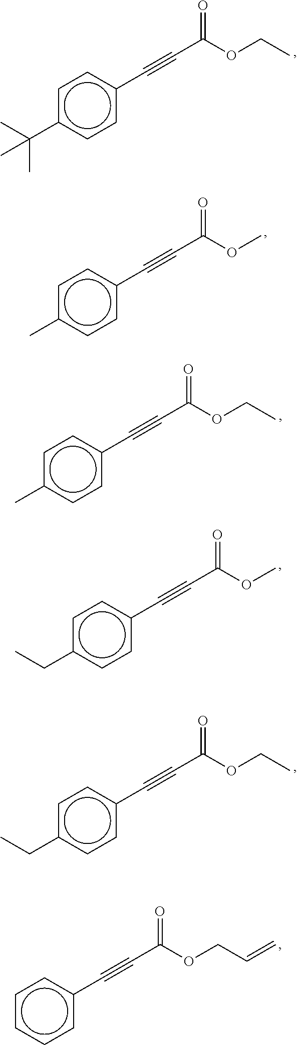

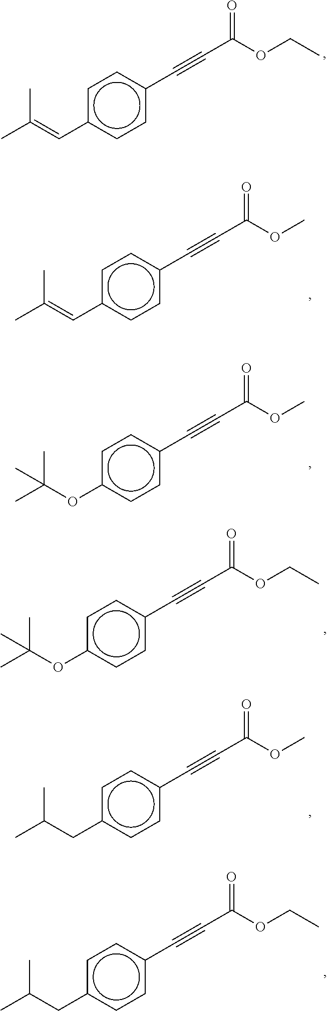

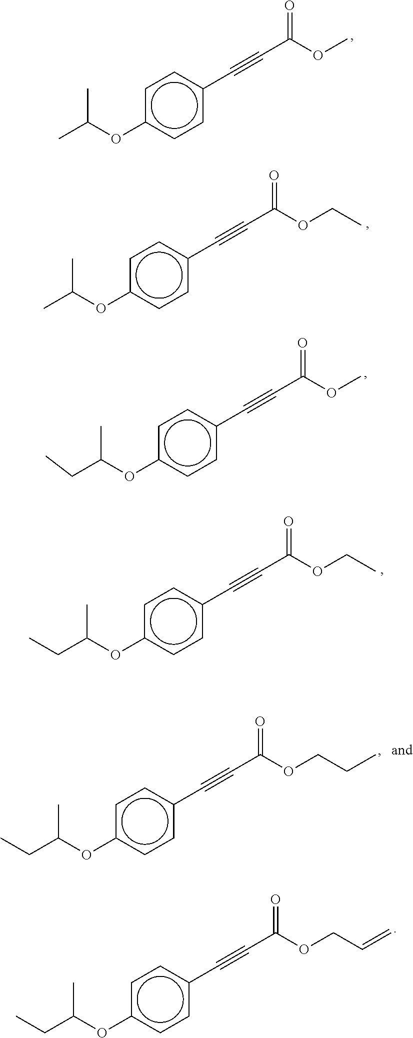

18. The composition of claim 3, wherein the compound is selected from the group consisting of:

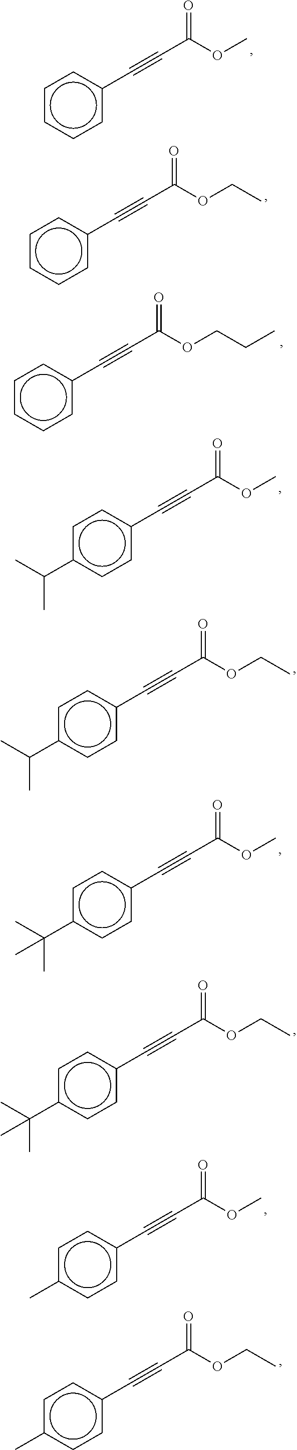

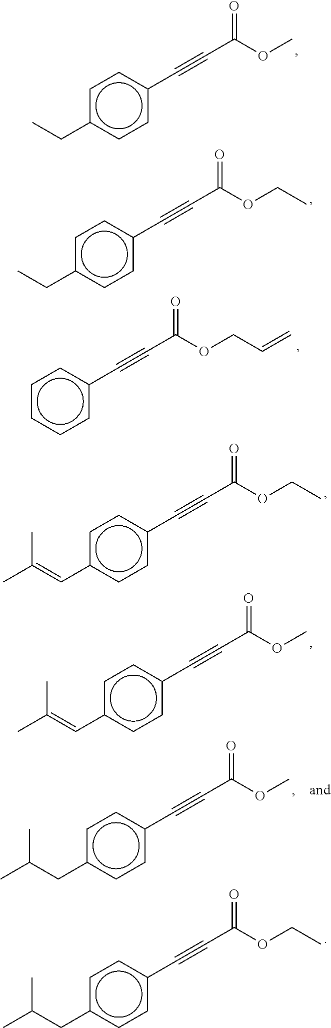

19. The composition of claim 3, wherein the compound is selected from the group consisting of: 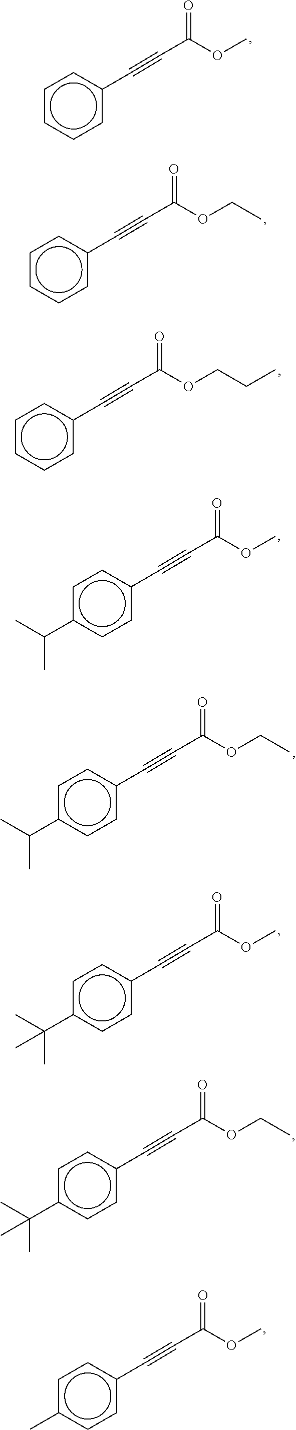 [chemical structure]
